(19) United States
(12) Patent Application Publication
(10) Pub. No.: US 2024/0408312 A1
(43) Pub. Date: Dec. 12, 2024

(54) METHOD OF MANUFACTURING A MEDICAL INJECTION DEVICE AND MEDICAL INJECTION DEVICE THUS OBTAINED

(71) Applicant: Stevanato Group S.p.A., Piombino Dese (PD) (IT)

(72) Inventors: Alberto Chillon, San Vito di Vigonza (PD) (IT); Fabio Chinellato, Treviso (IT); Paolo Patri, Paullo (MI) (IT)

(21) Appl. No.: 18/694,903

(22) PCT Filed: Sep. 26, 2022
(86) PCT No.: PCT/IB2022/059127

§ 371 Date: Mar. 22, 2024

(30) Foreign Application Priority Data
Sep. 24, 2021 (IT) 102021000024574
Mar. 1, 2022 (IT) 10202200003761

(51) Int. Cl. A61M 5/31; C03C 17/00; C03C 17/30

(52) U.S. Cl. CPC A61M 5/3129; C03C 17/004; C03C 17/30; A61M 2207/00; C03C 2218/11

(57) ABSTRACT

A method of manufacturing a medical injection device comprising a glass cylinder having an inner surface coated with a coating layer, the cylinder being configured to receive a plunger with sliding engagement includes the steps of providing a coating composition comprising an amount equal to or greater than 92% by weight of polydimethylsiloxane having a kinematic viscosity at room temperature of from 11500 cSt (115 cm²/s) to 13500 cSt (135 cm²/s); heating the coating composition to a temperature of from 100° C. to 150° C.; and applying the coating composition heated to said temperature onto the inner surface of the cylinder so as to form a coating layer on the inner surface having an average thickness S, measured by means of optical reflectometry, of from 100 to 250 nm; wherein the coating layer of the inner surface of the cylinder has a thickness standard deviation, equal to or less than 90 nm.

### CROSS REFERENCES

The application is a U.S. National Stage Application of International Application No. PCT/IB2022/059127, filed Sep. 26, 2022, which claims priority to Italian Application No. 102021000024574, filed Sep. 24, 2021, and Italian Application No. 102022000003761, filed Mar. 1, 2022, the disclosures of each of which are hereby expressly incorporated by reference herein in their entireties.

### TECHNICAL FIELD

The present disclosure relates to a method of manufacturing a medical injection device comprising a glass cylinder, having an inner surface coated with a coating layer, and configured to receive a plunger with sliding engagement, to a medical injection device obtained by means of said method and to a kit for assembling the aforesaid medical device.

### BACKGROUND

As is known, injection devices generally comprising a sealing plunger in sliding engagement within a container in order to dispense a drug by injection to a patient, are widely used in the medical field.

Such injection devices include syringes, cartridges but also self-injectors or automated injectors used for subcutaneous and/or intravenous administration of medications.

In this type of devices, a first need to be met is to have optimal sliding properties (in terms of static and dynamic friction) of the plunger within the cylinder of the injection device, e.g. of the cylinder of a syringe. To this end, a lubricating substance, typically based on silicone oil, is used to coat the inner surface of both the body of the syringe and the plunger. In particular, the objective of the lubricating substance used is to optimize the sliding properties of the plunger, in particular to obtain a low value of the force necessary to overcome the static friction (break-loose force) and of the force necessary to slide the plunger overcoming the dynamic friction (mean gliding force).

Another particularly felt need is to maintain the sliding properties of the plunger as constant as possible over time, in particular in the case of injection devices, for example syringes, pre-filled with a drug.

In fact, if one the one hand the use of pre-filled injection devices ensures a greater ease of administration of the drug and management flexibility, on the other hand it entails that the injection devices must be stored after filling for a rather long time, of the order of weeks or months, sometimes, such as for example in the case of protein-type drugs or vaccines, also at very low temperatures such as to guarantee the stability and a longer shelf life of the drug.

However, the presence of the silicone-based coating has been identified as one of the causes of instability of biotechnological drugs, in particular of recombinant proteins, an instability believed to be related to an intrinsic structural sensitivity. Silicone oil can in fact detach into a solution to form particles, in the literature classified as intrinsic particles, on which the proteins can be adsorbed at the silicone-water interface level, which proteins may undergo a structural denaturation and aggregation that can lead to the agglomeration of the particles themselves. The phenomenon of aggregation is critical as it results in a possible loss of efficacy of the therapeutic treatment and in an increased risk of immunogenicity.

In the case of pre-filled injection devices, therefore, a further important need arises, that is, that of maintaining over time not only optimal sliding properties of the coating, but also properties of low release of silicone particles within the pharmaceutical formulation.

### SUMMARY

The Applicant has noted that several methods of manufacturing a medical injection device have been proposed to try to meet these needs, which methods however trigger management or complexity problems and, therefore, cost issues, which have not been solved to date.

In some cases, mixtures of different types of silicone oils, possibly added with other substances, have been used. In this respect, the Applicant has noted that as one moves further away from pure silicone (i.e. unmixed or additive-free), the more difficult it is to maintain its properties and behaviour constant over time.

The irradiation of the silicone layer deposited on the inner surface of the syringe in order to crosslink, at least partially, the silicone has also been suggested: this has proven to be beneficial in achieving low values of particle release. Such irradiation may be by means of UV, IR, gamma rays, ion bombardment, or by means of a plasma treatment, under vacuum or at atmospheric pressure, of the torch or corona effect type.

In some cases, the deposition of several successive layers of silicone, possibly subjected to irradiation, has been proposed.

Examples of such processes, with silicones with additives, or mixed silicones, possibly with irradiation treatments, are described in US20020012741A1, EP3378514A1, U.S. Pat. No. 7,648,487B2, U.S. Pat. No. 9,662,450B2, U.S. Pat. No. 10,066,182B2, EP2387502B1, U.S. Pat. No. 7,553,529B2, US20110276005A1, EP2081615B1, U.S. Pat. Nos. 5,338,312A, 4,844,986A, 4,822,632A and US20080071228A1.

Methods aimed at obtaining a syringe that fulfils the two requirements of good sliding and low release, both of which constant over time (and also maintaining the thickness constant over time), are also described in WO2013045571A1. This document discloses the spraying of silicone with kinematic viscosity of from 900 to 1200 cSt onto the inner surface of the syringe and a subsequent plasma treatment to make the silicone stable and low-release. The document indicates the reason for the low release in the plasma treatment of the silicone surface.

A similar disclosure is provided by documents WO2009053947A2 and WO2015136037A1.

All these documents indicate the use of silicone with a rather low kinematic viscosity (around 1000 cSt), combined with an irradiation treatment, in particular a plasma one, as the best combination to solve the problems discussed above.

A method for internal siliconisation of hollow cylindrical bodies is also known from DE 100 00 505, in which silicone oil having preferably a kinematic viscosity of 350-20,000 cSt is deposited on the inner wall of the body cavity. The silicone oil is in particular deposited by spraying by means of a head of the type used in inkjet printing and which, in one embodiment, can be heated.

The Applicant has however observed that the manufacturing methods disclosed by the above-mentioned prior art, in addition to implying an undesired lengthening of the manufacturing times of the medical injection device and a greater management complexity of the method itself, trigger a further problem not identified by the prior art and related to the need to carry out a visual inspection of the medical injection device once filled with the drug in order to determine the absence of defects and extrinsic contaminants in the form of optically detectable particles.

This inspection, previously carried out manually, is now delegated to automated equipment based on techniques of analysis of images obtained from optical acquisition systems. The ever-increasing purity required to the solution contained in the medical injection device calls for a control that is capable not only to highlight even very small impurities present in the liquid, but also to discriminate them from cosmetic defects of the container which, however, do not constitute impurities and therefore, if erroneously classified as such, would lead to the rejection of the medical injection device.

In this regard, the Applicant has observed that the partial cross-linking of the layer of silicone oil applied to the inner surface of the cylinder of the medical injection device, in particular obtained by plasma irradiation, produces a more irregular, albeit more stable, surface structure that can mislead an automated optical inspection system, erroneously categorising the surface irregularity as impurity and thus generating production waste that has no reason to exist with a consequent economic damage.

The Applicant has therefore perceived that it is necessary to develop a method of manufacturing a medical injection device that allows not only to satisfy the aforesaid needs of having optimal sliding properties (in terms of static and dynamic friction) of the plunger within the cylinder of the injection device and optimal properties of low release of particles, both constant over time, but that is also able to reduce the problems related to false defects that can be erroneously detected by the visual inspection devices of the medical injection device.

The Applicant has understood that all these desired features can be accomplished by acting on the rheological characteristics of the coating composition and on the application methods of the coating composition used to coat the inner surface of the cylinder of the medical injection device compared to what is suggested by the prior art.

In particular, the Applicant has experimentally verified that by using to coat the inner surface of the cylinder of the medical injection device a coating composition constituted substantially for almost the totality thereof by a single type of silicone oil having a kinematic viscosity at room temperature much higher than that of the silicone oil suggested by the prior art and by heat-applying this silicone oil on the inner surface of the cylinder it is possible, after cooling of the coating layer applied to this surface, to simultaneously obtain:
the desired optimal sliding and low particle release properties, both substantially constant over time, and
optimal characteristics of surface regularity of the coating layer, such that the visual inspection devices of the medical injection device are not misled.

In particular, the aforesaid characteristics of surface regularity of the coating layer were experimentally comparable to those of the non-crosslinked coating layers, obtained by using a silicone oil, having a low kinematic viscosity but a high particle release, of the prior art. And this, despite the use of a silicone oil having a significantly higher kinematic viscosity at room temperature and despite the fact that the applied coating layer has very low average thicknesses, of the order of 100-250 nm.

The aforesaid characteristics of surface regularity of the coating layer were, however, experimentally improved compared to the partially cross-linked coating layers of the prior art obtained by using low kinematic viscosity silicone oil.

Furthermore, the Applicant has experimentally verified that by using to coat the inner surface of the cylinder of the medical injection device the aforesaid coating composition constituted substantially for almost the totality thereof by a single type of silicone oil having a kinematic viscosity at room temperature much higher than that of the silicone oil suggested by the prior art, and by heat-applying this silicone oil on the inner surface of the cylinder, it is also possible to obtain characteristics of coating uniformity with a high process repeatability as required in large-scale industrial productions.

Thus, the present invention relates, in a first aspect thereof, to a method of manufacturing a medical injection device comprising a glass cylinder having an inner surface coated with a coating layer and configured to receive a plunger with sliding engagement, as defined in the appended claims 1 and 2.

In particular, in a first embodiment thereof, the method of manufacturing a medical injection device according to the invention comprises the steps of:
a) providing a coating composition comprising an amount equal to or greater than 92% by weight of polydimethylsiloxane having a kinematic viscosity at room temperature of from 11500 cSt (115 cm²/s) to 13500 cSt (135 cm²/s);
b) heating the coating composition to a temperature of from 100° C. to 150° C.;
c) applying the coating composition heated to said temperature onto the inner surface of the cylinder so as to form a coating layer having an average thickness S, measured by optical reflectometry, of from 100 to 250 nm on said inner surface;
   wherein the coating layer of the inner surface of the cylinder has a thickness standard deviation, equal to or less than 90 nm.

Furthermore, in a second embodiment thereof, the method of manufacturing a medical injection device according to the invention comprises the steps of:
a) providing a coating composition comprising an amount equal to or greater than 92% by weight of polydimethylsiloxane having a kinematic viscosity at room temperature of from 11500 cSt (115 cm²/s) to 13500 cSt (135 cm²/s);
b) heating the coating composition to a temperature of from 100° C. to 150° C.;
c) applying the coating composition heated to said temperature onto the inner surface of the cylinder so as to form a coating layer having an average thickness, measured by optical reflectometry, of from 100 to 250 nm on said inner surface:
   wherein for each batch of 10 cylinders the batch average standard deviation SD of the thickness of the coating layer has a value equal to or less than 70 nm;
   wherein the batch average standard deviation SD is obtained by:
   measuring the thickness Sₚᵢ of the coating layer in at least 6 points of each arbitrary portion ni of an i-th cylinder of the batch having an axial length of 1.0 mm and developed in plane;
   ii) calculating, for each of the aforesaid portions ni of the i-th cylinder of the batch, and for each i-th cylinder, the average thickness Sₙᵢ by means of the formula

$${S}_{ni} = ( {\sum }_{p = 1 , 6} {S}_{pi} ) / 6$$

iii) calculating, for each cylinder portion n, the batch average thickness of the portion n S_{nL} by means of the formula

$${S}_{nL} = ( {\sum }_{i = 1 , 10} {S}_{ni} ) / 10$$

iv) calculating, for the 10 syringes of the batch, a standard deviation SDₙ with respect to the batch average thickness of the portion n S_{nL}, and
   v) calculating the batch average standard deviation SD from the values of the thickness standard deviation SDₙ, by means of the formula

$$SD = ( {\sum }_{i = 1 , N} {SD}_{n} ) / N$$

where N is the total number of portions n of each cylinder of the batch.

The Applicant has experimentally found, as will be explained in more detail below, that by heat-applying the aforesaid coating composition based on polydimethylsiloxane with high viscosity at room temperature, it is possible to form on the inner surface of the cylinder a coating layer with the same effectiveness, in terms of application and distribution, of an oil with lower viscosity.

The Applicant has also experimentally found that the coating layer, after cooling and after its viscosity characteristics have returned to those present at room temperature, achieves a series of advantageous improved characteristics as compared to the coating layers with lower viscosity, whether or not they are subjected to partial cross-linking, described by the prior art.

Firstly, the Applicant has experimentally observed that the method of the invention advantageously allows to form a coating layer having not only the low thickness values that are required by the pharmaceutical and cosmetic industry, but also a very homogeneous distribution on the inner surface and along each section of the cylinder.

In particular, the Applicant has experimentally observed that the method of the invention advantageously allows to apply on the inner surface of the cylinder a coating layer having thickness values that are fully comparable to those obtainable using low viscosity silicone oils suggested by the prior art.

The Applicant has experimentally observed that the viscosity of the coating layer applied to the inner surface of the cylinder, once returned to its value at room temperature, confers to the layer such stability characteristics which allow to overcome all the drawbacks of the coating layers formed by silicone oils with lower viscosity (of the order, as mentioned, of about 1000 cSt) and not subjected to partial cross-linking.

In particular, the method of the invention advantageously allows to form a coating layer which overcomes the following drawbacks of the non-crosslinked coating layers of the prior art:
tendency of the silicone layer to develop over time a non-uniformity in the distribution along the axis of the cylinder of the medical injection device, for example of a syringe, due to the migration by gravity of the silicone towards the lower portion of the cylinder body during storage in an upright position;
consequent unevenness of the sliding resistance of the plunger when the medical injection device, e.g. a syringe, is used;
consequent greater likelihood of direct interaction of the drug with the material (glass) which the cylinder of the medical injection device, e.g. a syringe, is made of, and of detachment of portions of the coating layer from the surface into the solution; and
possibility of triggering denaturation and protein aggregation phenomena, especially if combined with mechanical stresses such as stirring or while dispensing the liquid present in the cylinder which occurs by sliding the plunger.

The method of the invention therefore advantageously allows to form a coating layer having thickness, uniformity and stability characteristics that allow to achieve optimal sliding characteristics of the plunger in the cylinder, although this layer is formed by a silicone oil with a much higher viscosity than that suggested by the prior art documents discussed above.

Secondly, the Applicant has experimentally observed that the method of the invention advantageously allows to form a coating layer having a high surface regularity and a high uniformity of coverage, such that visual inspection devices of the medical injection device, in particular those of the automated type, are not misled.

In particular, the method of the invention advantageously allows to obtain a coating layer on the inner surface of the cylinder having a very uniform thickness with a thickness standard deviation, measured by optical reflectometry (or optical interferometry depending on the resolution), equal to or less than 90 nm.

In this way, the coating layer does not trigger problems of false defects, thus solving the problem observed with the partially cross-linked silicone coatings of the prior art.

Advantageously, the method of the invention also allows to obtain a coating layer on the inner surface of the cylinder having an average thickness completely in line with the demands of the pharmaceutical and cosmetic industry despite the fact that such a coating layer is constituted by a silicone material with high kinematic viscosity.

Thirdly, the Applicant has experimentally observed that the method of the invention advantageously allows to form a coating layer having, thanks to its stability characteristics related to the viscosity values at room temperature of the coating layer, characteristics of low particle release in the solution stored in the cylinder of the medical injection device.

According to the tests carried out by the Applicant, these characteristics of low particle release are entirely comparable or improved compared to those of the partially cross-linked silicone coating layers of the prior art which nevertheless trigger the problems of false defects mentioned above.

Fourthly, the Applicant has experimentally observed that the aforesaid characteristics of optimal sliding of the plunger and of low particle release in the solution stored in the cylinder remain substantially constant over time, both in the case of storages at room temperature or above room temperature, and in the case of storages at low temperature, so as to satisfy another important demand of the pharmaceutical and cosmetic industry.

Fifthly, the Applicant has experimentally observed that the aforesaid characteristics of uniformity of the average thickness of the coating layer can be obtained in a highly repeatable manner within different production batches of the medical device, a highly desirable characteristic within the large-scale productions typical of the pharmaceutical and cosmetic industry. And, this, despite the fact that this coating layer is constituted by a silicone material with high kinematic viscosity.

In a further aspect thereof, the present invention relates to an apparatus for manufacturing a medical injection device comprising a glass cylinder having an inner surface coated with a coating layer and configured to receive a plunger with sliding engagement, as defined in the appended claim 25.

In particular, the apparatus for manufacturing a medical injection device according to the invention comprises:
a storage tank of a coating composition provided with at least one heating element configured to heat the stored coating composition;
at least one dispensing head configured to dispense the heated coating composition and provided with at least one dispensing nozzle, the dispensing head being provided with a respective heating element configured to heat the coating composition dispensed by the nozzle;
a circulation pump arranged upstream of the dispensing head;
a supporting frame of one or more cylinders of respective medical injection devices;
wherein said at least one dispensing head and the supporting frame are movable relative to each other for inserting/extracting the nozzle of said at least one dispensing head in a respective cylinder of said one or more cylinders.

In particular, according to a first embodiment, the medical injection device according to the invention comprises a glass cylinder having an inner surface coated with a coating layer, the cylinder being configured to receive a plunger with sliding engagement, wherein said coating layer of the inner surface of the cylinder is substantially made of polydimethylsiloxane having a kinematic viscosity at room temperature of from 11500 cSt (115 cm²/s) to 13500 cSt (135 cm²/s) and has an average thickness of from 100 to 250 nm; and wherein the coating layer of the inner surface of the cylinder has a thickness standard deviation, equal to or less than 90 nm.

Furthermore, according to a second embodiment, the medical injection device according to the invention comprises a glass cylinder having an inner surface coated with a coating layer, the cylinder being configured to receive a plunger with sliding engagement, wherein said coating layer of the inner surface of the cylinder is substantially made of polydimethylsiloxane having a kinematic viscosity at room temperature of from 11500 cSt (115 cm²/s) to 13500 cSt (135 cm²/s) and has an average thickness of from 100 to 250 nm; wherein for each batch of 10 cylinders the batch average standard deviation SD of the thickness of the coating layer has a value equal to or less than 70 nm: wherein the batch average standard deviation SD is obtained by:
measuring the thickness Sₚᵢ of the coating layer in at least 6 points of each arbitrary portion ni of an i-th cylinder of the batch having an axial length of 1.0 mm and developed in plane;
ii) calculating, for each of the aforesaid portions ni of the i-th cylinder of the batch, and for each i-th cylinder, the average thickness Sₙᵢ by means of the formula

$${S}_{ni} = ( {\sum }_{p = 1 , 6} {S}_{pi} ) / 6$$

iii) calculating, for each n portion of cylinder, the batch average thickness of the portion n S_{nL} by means of the formula

$${S}_{nL} = ( {\sum }_{i = 1 , 10} {S}_{ni} ) / 10$$

iv) calculating, for the 10 syringes of the batch, a standard deviation SDₙ with respect to the batch average thickness of the portion n S_{nL}, and
v) calculating the batch average standard deviation SD from the values of the thickness standard deviation SDₙ, by means of the formula

$$SD = ( {\sum }_{i = 1 , N} {SD}_{n} ) / N$$

where N is the total number of portions n of each cylinder of the batch.

Advantageously, the aforesaid injection device achieves the advantageous technical characteristics illustrated above with reference to the method of its manufacture and related to the characteristics achieved by the coating layer of the inner surface of the cylinder.

In particular, according to a first embodiment, the kit of parts according to the invention comprises the following separate components in a sterile package:
a glass cylinder having an inner surface coated with a coating layer, the cylinder being configured to receive a plunger with sliding engagement,
a plunger configured for a sliding engagement in said cylinder,
wherein said coating layer of the inner surface of the cylinder is substantially made of polydimethylsiloxane having a kinematic viscosity at room temperature of from 11500 cSt (115 cm²/s) to 13500 cSt (135 cm²/s) and has an average thickness S of from 100 to 250 nm; and wherein the coating layer of the inner surface of the cylinder has a thickness standard deviation, measured by optical reflectometry, equal to or less than 90 nm.

Furthermore, according to a second embodiment, the kit of parts according to the invention comprises the following separate components in a sterile package:
a glass cylinder having an inner surface coated with a coating layer, the cylinder being configured to receive a plunger with sliding engagement,
a plunger configured for a sliding engagement in said cylinder,
wherein said coating layer of the inner surface of the cylinder is substantially made of polydimethylsiloxane having a kinematic viscosity at room temperature of from 11500 cSt (115 cm²/s) to 13500 cSt (135 cm²/s) and has an average thickness of from 100 to 250 nm;
wherein for each batch of 10 cylinders the batch average standard deviation SD of the thickness of the coating layer has a value equal to or less than 70 nm;
wherein the batch average standard deviation SD is obtained by;
measuring the thickness Sₚᵢ of the coating layer in at least 6 points of each arbitrary portion ni of an i-th cylinder of the batch having an axial length of 1.0 mm and developed in plane;
ii) calculating, for each of the aforesaid portions ni of the i-th cylinder of the batch, and for each i-th cylinder, the average thickness Sₙᵢ by means of the formula

$${S}_{ni} = ( {\sum }_{p = 1 , 6} {S}_{pi} ) / 6$$

iii) calculating, for each cylinder portion n, the batch average thickness of the portion n S_{nL} by means of the formula

$${S}_{nL} = ( {\sum }_{i = 1 , 10} {S}_{ni} ) / 10$$

iv) calculating, for the 10 syringes of the batch, a standard deviation SDₙ with respect to the batch average thickness of the portion n S_{nL}, and
v) calculating the batch average standard deviation SD from the values of the thickness standard deviation SDₙ, by means of the formula

$$SD = ( {\sum }_{i = 1 , N} {SD}_{n} ) / N$$

where N is the total number of portions n of each cylinder of the batch.

Advantageously, the aforesaid kit of parts allows to store and transport in a sterile manner and subsequently assemble the injection device disclosed herein.

### Definitions

Within the framework of the present description and in the subsequent claims, the term “room temperature” (RT) indicates a temperature of 25°±2° C. measured at a relative humidity of 60%.

Within the framework of the present description and in the subsequent claims, all percentages are understood as % by weight where specifically indicated.

In the context of the description and of the subsequent claims, the term “average value” refers to the arithmetic mean of the values of the specific entity considered.

Within the framework of the present description and in the subsequent claims, all the pressure values are to be understood as relative pressure values. In other words, the pressure values indicated in the present document do not include the pressure of the weight of the atmosphere unless otherwise specified.

Within the framework of the present description and in the subsequent claims, all numerical entities indicating amounts, parameters, percentages, and so on are to be understood as preceded in all circumstances by the term “about” unless otherwise indicated. In addition, all the ranges of numerical entities include all the possible combinations of the maximum and minimum numerical values and all the possible intermediate ranges, in addition to those specifically indicated below.

Within the framework of the present description and in the subsequent claims, the kinematic viscosity of polydimethylsiloxane was measured by means of TGA and DSC thermo-gravimetric techniques.

Thermogravimetry (TG) or thermogravimetric analysis (TGA) is an experimental technique for characterizing materials falling within the wider family of thermal analysis. The technique consists in the continuous measurement over time of the mass variation of a material sample as a function of time (isotherm) or of temperature (heating/cooling ramp), under controlled atmosphere conditions.

The DSC technique allows to determine at what temperature—or range of temperatures—any transitions occur (for example melting or crystallization processes) and to quantitatively measure the energies associated thereto. DSC analysis in fact measures the heat flows that occur in a sample when it is heated/cooled (dynamic conditions) or maintained at a constant temperature (isothermal conditions) in a controlled manner.

By coupling these two techniques, it is possible to determine the kinematic viscosity of a silicone material by correlating the thermal curves obtained with the standard ones of silicone oil with known viscosity.

In this way, it is possible to determine the kinematic viscosity of a silicone material using a calibration curve capable of correlating the viscosity values (related to the length of the polymer chain) to the thermal phenomena (weight loss) observed at different temperatures.

The polydimethylsiloxane present in the coating layer is extracted with multiple aliquots of dichloromethane which was evaporated before analysis.

TGA analysis was performed using a TGA 4000 thermogravimetric analyser (PerkinElmer), while DSC analysis was performed using a DSC 204 F1 differential scanning calorimeter (Netzsch).

The thermal cycle followed for the TGA analysis was: from 30° C. to 500° C., with a heating ramp of 10° C./min.

The thermal cycle followed for the DSC analysis was: from −80° C. to 30° C., with a heating ramp of 10° C./min.

Within the framework of the present description and in the subsequent claims, the thickness of the coating layer applied to the inner surface of the cylinder of the injection device is to be understood as measured by optical techniques based on the emission of a light radiation (white light or of a specific wavelength by laser) that collides on the analysis sample.

The instrument, such as for example an optical reflectometer, detects the difference of the reflected wavelength of two beams of light, one reflected by the material (glass) of the cylinder of the injection device and one by the coating layer. This difference allows the thickness of the layer to be determined by knowing the refractive index and the geometry of the sample analysed. If a white light is used as a light source during the analysis, the instrument may detect minimum thicknesses of 80 nm. By using a specific collimated wavelength (laser), for example a collimated wavelength of 630-680 nm, the resolution can increase up to 20 nm, in this case being able to use interferometric techniques.

Within the framework of the present description and in the subsequent claims, the average thickness S of the coating layer is in particular and preferably obtained by:
measuring the thickness Sₚ of the coating layer in at least 6 points of each arbitrary portion n of cylinder having an axial length of 1.0 mm and developed in plane,
ii) calculating the average thickness Sₙ of each of the aforesaid n portions of cylinder, where Sₙ=(Σ_{p=1,6} Sₚ)/6,
iii) calculating, the average thickness S of the coating layer of the cylinder, where S=(Σ_{n=1,N} Sₙ)/N, and N is the total number of portions n of the cylinder.

In general, within the framework of the present description and in the subsequent claims, the term “standard deviation” or “average square deviation” of an entity “x”, for example the thickness of the coating layer applied to the inner surface of the cylinder of the injection device, as detected on a population of N statistical units is defined as:

$${σ}_{X} = \sqrt{\frac{{\sum }_{i = 1}^{N} {( {x}_{i} - \overline{x} )}^{2}}{N}} ,$$

$$where$$

$$\overline{x} = \frac{1}{N} {\sum }_{i = 1}^{N} {x}_{i}$$

is the arithmetic mean of the entity “x”

In particular and preferably, the thickness standard deviation of the coating layer applied to the inner surface of the cylinder of the injection device is obtained by determining the average thickness S of the coating layer according to points i)-iii) referred to above and by iv) calculating a standard deviation SD of the average thicknesses Sₙ of the aforesaid n portions of cylinder with respect to the average thickness S of the coating layer of the cylinder.

Within the framework of the embodiments of the invention, the average thickness of the coating layer applied to the inner surface of each cylinder of a batch of predetermined number of cylinders, for example 10, and the batch standard deviation of the coating layer are obtained as indicated above.

Within the framework of the embodiments of the invention, “batch average standard deviation SD of the thickness of the coating layer” means the arithmetic mean of the thickness standard deviation SDₙ obtained as indicated above. As indicated above, this parameter is indicative of the process repeatability between the various production batches.

Within the framework of all embodiments of the invention, the total number of the n portions having an axial length of 1.0 mm and developed in plane of the injection device cylinder, indicated by N, varies as a function of the size of the cylinder itself.

Thus, for example, the total number N of the n portions of the injection device is equal to 40 in the case of a syringe of nominal volume of 0.5 mL, 45 in the case of a syringe of nominal volume of 1.0 mL Long and 90 in the case of a syringe of nominal volume of 3.0 mL.

Within the framework of the present disclosure and in the subsequent claims, the designations of syringes with nominal volume of 0.5 mL, 1 mL long or 3 mL are intended according to the standard ISO 11040-4 (2015).

Within the framework of the present description and in the subsequent claims, the term “axial” and the corresponding term “axially” are used to refer to a longitudinal direction of the medical injection device, which corresponds to the longitudinal direction of its cylinder, whereas the term “radial” and the corresponding term “radially” are used to refer to any direction perpendicular to the aforementioned longitudinal direction.

Within the framework of the present description and in the subsequent claims, the term “circumferential” and the corresponding term “circumferentially” are used to refer to a direction of development of the inner surface of the cylinder of the medical injection device in a plane perpendicular to the longitudinal direction of the cylinder itself.

The present invention can have, in one or more of the aforementioned aspects, one or more of the preferred features set forth below, which can be combined as desired with each other according to the application requirements.

In a preferred embodiment, step a) comprises providing a coating composition comprising an amount equal to or greater than 95% by weight, more preferably equal to or greater than 98% by weight, of polydimethylsiloxane having a kinematic viscosity at room temperature of from 11500 cSt (115 cm²/s) to 13500 cSt (135 cm²/s).

Even more preferably, step a) comprises providing a coating composition comprising an amount equal to about 100% by weight of polydimethylsiloxane having a kinematic viscosity at room temperature of from 11500 cSt (115 cm²/s) to 13500 cSt (135 cm²/s).

In this way, it is advantageously possible to have a manufacturing method that can be implemented in a particularly simple and repeatable way by minimizing or completely eliminating the problems related to the difficulty of maintaining the rheological properties of the coating composition constant after mixing silicone materials with different density and/or viscosity.

Advantageously, the manufacturing method can also be implemented without any addition of additives to the silicone material.

In a preferred embodiment, step a) of providing the coating composition comprises storing said coating composition in a storage tank.

In this way, it is advantageously possible to always have the desired amounts of coating composition available for the implementation of the method.

Preferably, the tank is made of a material suitable for containing the silicone coating composition, e.g. stainless steel.

Preferably, step b) provides for heating the coating composition to a temperature of from 120° C. to 150° C.

In this way, it is advantageously possible to optimise the subsequent step c) of applying the heated coating composition onto the inner surface of the cylinder, thereby facilitating the formation of a very uniform coating layer on the inner surface.

In a preferred embodiment, step b) of heating the coating composition comprises heating the aforesaid storage tank so as to bring the coating composition to said temperature of from 100° C. to 150° C. and, more preferably, of from 120° C. to 150° C.

To this end, the storage tank of a coating composition is provided with at least one heating element configured to heat the stored coating composition.

For the purposes of the invention, the heating element of the tank can be any element configured to release thermal energy and selectively placed in heat exchange relationship with the coating composition stored in the storage tank.

Merely by way of example, the heating element may be a heating coil (and e.g. an electrical resistor or a pipe in which a suitable heating fluid circulates) placed inside the tank, or a jacket outside the tank in which one or more electrical resistors are placed or in which a suitable heating fluid circulates.

In a preferred embodiment, the method may further comprise a step d) of maintaining the heated coating composition stored in the storage tank at a pressure of from 5 psi (0.34 bar) to 150 psi (10.34 bar), preferably of from 10 psi (0.69 bar) to 30 psi (2.07 bar), even more preferably of from 10 psi (0.69 bar) to 15 psi (1.03 bar).

In this way, it is advantageously possible to optimise the subsequent step c) of applying the heated coating composition onto the inner surface of the cylinder, thereby facilitating the formation of a very uniform coating layer on the inner surface.

In a preferred embodiment, the method further comprises a step e) of feeding the heated coating composition to a dispensing head provided with at least one dispensing nozzle.

In this way, it is advantageously possible to apply the heated coating composition on the inner surface of the cylinder so as to form a very uniform coating layer on the inner surface.

Preferably, the dispensing head of the heated coating composition is provided with a respective heating element configured to heat the coating composition dispensed by the nozzle.

For the purposes of the invention, the heating element of the nozzle may be any element configured to release thermal energy selectively placed in heat exchange relationship with the coating composition being dispensed by the nozzle itself.

Merely by way of example, the heating element may be an electrical resistor in heat exchange relationship with the dispensing nozzle, for example incorporated in a casing, for example cylindrical, associated to the dispensing nozzle.

Preferably, step e) of feeding the heated coating composition to the dispensing head is carried out by means of a circulation pump arranged upstream of the dispensing head.

In this way, it is advantageously possible to appropriately feed the dispensing head of the coating composition according to the production needs.

In a preferred embodiment, the circulation pump comprises a respective heating element configured to heat a delivery head of the pump.

For the purposes of the invention, the heating element of the delivery head of the pump may be any element configured to release thermal energy selectively placed in heat exchange relationship with the coating composition being dispensed by the delivery head itself.

Merely by way of example, the heating element may comprise one or more electrical resistors in heat exchange relationship with the delivery head of the pump, for example incorporated in a respective casing, for example cylindrical, associated to the delivery head.

In a preferred embodiment, step c) of applying the heated coating composition onto the inner surface of the cylinder is carried out by dispensing the coating composition via the dispensing head.

In this way, it is advantageously possible to apply the heated coating composition in a very uniform manner on the inner surface of the cylinder.

In a preferred embodiment, step b) of heating the coating composition comprises heating the dispensing head and/or the pump, more preferably the delivery head of the pump, so as to bring or maintain the coating composition at/to said temperature of from 100° C. to 150° C.

In this way, it is advantageously possible to reduce the power absorption and the wear of the pump to the benefit of the operating and maintenance costs of the same.

In preferred embodiments, the dispensing head and the pump may be heated as described above.

In preferred embodiments, the manufacturing method provides for heating the delivery head of the pump to a temperature of from 50° C. to 60° C.

In a preferred embodiment, the storage tank of the coating composition, the circulation pump and the dispensing head are in fluid communication with each other via pipes.

Preferably, the pipes are in heat exchange relationship with a respective heating element for example an electrical resistor or an outer jacket of the pipes in which a suitable heating fluid circulates.

Preferably, the aforesaid pipes are made of a temperature-resistant material, such as stainless steel, and thermally insulated, or made of a thermally insulating, metal or plastic material.

The Applicant has experimentally observed that by carrying out a heating of one or more among the storage tank of the coating composition, the circulation pump, the dispensing head and the respective connection pipes it is advantageously possible to equalize the viscosity of the coating composition before it is dispensed on the inner surface of the cylinder with a consequent advantageous reduction in the dispensing time and a greater distribution uniformity of the coating composition on the inner surface of the cylinder.

In the context of this preferred embodiment, step b) of heating the coating composition preferably comprises heating the aforesaid pipes so as to bring or maintain the coating composition at/to the aforesaid temperature of from 100° C. to 150° C.

The Applicant has experimentally observed that heating the coating composition to a temperature above 150° C. may result in a change in the properties of the silicone material which may lead to undesired increased particle release and/or release of substances that at lower temperatures are normally retained.

In a preferred embodiment, step c) of applying the heated coating composition onto the inner surface of the cylinder is carried out by dispensing the heated coating composition at a pressure of from 5 psi (0.34 bar) to 150 psi (10.34 bar), more preferably of from 6 psi (0.41 bar) to 10 psi (0.69 bar).

In this way, it is advantageously possible to apply the heated coating composition in a very uniform manner on the inner surface of the cylinder.

In a preferred embodiment, step c) of applying the heated coating composition onto the inner surface of the cylinder comprises feeding to the dispensing head a dispensing gas (e.g. air) having a pressure of from 5 psi (0.34 bar) to 150 psi (10.34 bar), preferably of from 6 psi (0.41 bar) to 10 psi (0.69 bar).

In this way, it is advantageously possible to dispense the heated coating composition in a very uniform manner so as to apply an equally uniform coating layer on the inner surface of the cylinder.

In a preferred embodiment, the method comprises maintaining the storage tank of the coating composition at a pressure higher than the pressure of the dispensing nozzle of the dispensing head.

In this way, it is advantageously possible to dispense the heated coating composition in a very uniform manner so as to apply an equally uniform coating layer on the inner surface of the cylinder.

In a preferred embodiment, step c) of applying the heated coating composition onto the inner surface of the cylinder comprises imparting a relative motion between the dispensing head and the cylinder while dispensing the heated coating composition.

In a preferred embodiment, step c) of applying the heated coating composition onto the inner surface of the cylinder comprises dispensing the heated coating composition onto the inner surface of the cylinder during a relative insertion movement of the dispensing head into the cylinder.

In preferred embodiments, one or more cylinders of respective medical injection devices may be supported by a movable supporting frame relative to one or more respective dispensing heads of the heated coating composition.

In this way, it is thus possible to insert/extract the nozzle of the dispensing head(s) in a respective cylinder of said one or more cylinders.

Preferably, the dispensing head(s) are fixed and the supporting frame of said one or more cylinders is movable towards and from the dispensing head(s) so as to facilitate the implementation of the relative movement between the latter and the cylinder(s).

In alternative preferred embodiments, the dispensing head(s) may be movable and the supporting frame of said one or more cylinders may be fixed, or again the dispensing head(s) and the supporting frame may both be movable.

Preferably, step c) of applying the heated coating composition onto the inner surface of the cylinder comprises dispensing the coating composition by means of the nozzle of the dispensing head while moving the cylinder(s) towards the respective dispensing head(s).

In this way, it is advantageously possible to apply a very uniform coating layer on the inner surface of the cylinder.

In a preferred embodiment, the dispensing time of the heated coating composition onto the inner surface of the cylinder is of from 0.3 s to 1 s, more preferably of from 0.4 s to 0.7 s.

In this way, it is advantageously possible to limit the so-called “total cycle time” or “spraying time” given by the sum of the times of insertion and extraction of the dispensing head into and from the cylinder to values of less than about 3 s, considered compatible with the normal cycle times of an industrial production line.

In this regard, the Applicant has experimentally observed that the aforesaid dispensing times of the heated coating composition can be advantageously and conveniently achieved by implementing one or more of the aforesaid steps of heating the storage tank, heating the dispensing head, heating the circulation pump arranged upstream of the dispensing head or parts of said pump (e.g. and preferably the delivery head of the pump) and heating the connecting pipes which ensure a fluid communication between the storage tank, the pump and the dispensing head.

In a particularly preferred embodiment, the above-mentioned dispensing times of the heated coating composition are advantageously and conveniently achieved by implementing the steps of heating the storage tank, the pump, the dispensing head and the related connection pipes.

As explained above, the Applicant has in fact experimentally observed that by operating in this way it is possible to equalize the viscosity of the coating composition before the same is dispensed onto the inner surface of the cylinder with a consequent advantageous reduction in the dispensing time and a greater distribution uniformity of the coating composition on the inner surface of the cylinder.

In a preferred embodiment, step c) of applying the heated coating composition onto the inner surface of the cylinder comprises dispensing the heated coating composition at a flow rate of from 0.1 μL/s to 5 μL/s, more preferably equal to about 0.5 μL/s.

In this way, it is advantageously possible to apply a very thin coating layer on the inner surface of the cylinder.

In a preferred embodiment, step c) of applying the heated coating composition onto the inner surface of the cylinder comprises applying to the inner surface of the cylinder an amount per unit area of heated coating composition of from 0.2 to 0.4 μg/mm².

Also in this case, it is advantageously possible to apply a very thin coating layer on the inner surface of the cylinder.

In a preferred embodiment, step c) of applying the heated coating composition onto the inner surface of the cylinder is carried out such that the coating layer formed on the inner surface of the cylinder has an average thickness, measured by optical reflectometry, of from 100 to 200 nm.

Advantageously and as explained above, this average thickness of the coating layer formed on the inner surface of the cylinder is completely in line with the demands of the pharmaceutical and cosmetic industry despite the fact that the coating layer is constituted by a silicone material with high kinematic viscosity.

In a preferred embodiment, the method of the invention allows to obtain a coating layer formed on the inner surface of the cylinder having a very uniform thickness having a thickness standard deviation, measured by optical reflectometry (or optical interferometry depending on the resolution), equal to or less than 70 nm, and, even more preferably, equal to or less than 50 nm.

In this way, it is advantageously possible to obtain a coating layer having optimal characteristics of surface regularity and such that the visual inspection devices of the medical injection device, in particular those of the automated type, are not misled.

In a preferred embodiment, the method of the invention allows to obtain for each batch of 10 cylinders a coating layer formed on the inner surface of the cylinder having a very uniform thickness and such that the batch average standard deviation SD of the thickness of the coating layer, as defined above, has a value equal to or less than 60 nm, and, even more preferably, equal to or less than 50 nm.

In this way, it is advantageously possible to obtain a coating layer having optimal characteristics of surface regularity in a very reproducible way on several cylinders of a batch as required in large-scale industrial productions.

In a preferred embodiment, the method of manufacturing the medical injection device according to the invention may further comprise, after step c) of applying the heated coating composition onto the inner surface of the cylinder, a step f) of subjecting the coating layer formed on the inner surface of the cylinder to a partial cross-linking treatment of the polydimethylsiloxane.

Preferably, the partial cross-linking treatment is carried out by irradiation.

Preferably, the irradiation treatment of the coating layer is a plasma irradiation treatment, preferably an irradiation treatment by means of plasma torch at atmospheric pressure with argon flow preferably with purity greater than 99% (e.g. 99.999%).

In this way, it is advantageously possible—if desired depending on the specific application—to further improve the characteristics of low particle release of the coating layer.

Advantageously, the Applicant has experimentally found that the partial cross-linking treatment can be carried out such that the lubrication characteristics of the coating layer are not penalized.

To this end, in preferred embodiments, said irradiation treatment is carried out for a time of from 0.2 s to 1 s, preferably of from 0.2 to 0.6 s, more preferably of from 0.2 to 0.5 s, extremes included, and, even more preferably, equal to about 0.3 s.

The Applicant has experimentally found, as will be explained in more detail below, that by limiting the irradiation time in this range of values it is advantageously possible to obtain a coating layer having optimal sliding properties (in terms of static and dynamic friction) of the plunger within the cylinder of the injection device and at the same time optimal properties of a low particle release, both constant over time.

Advantageously, the partially cross-linked coating layer obtained according to this preferred embodiment still remains capable, thanks to its surface regularity, to substantially reduce the problems related to false defects that can be erroneously detected by the visual inspection devices of the medical injection device, in particular by those of the automated type.

Without wishing to be bound by any interpretative theory, the Applicant considers that an irradiation time falling within the aforesaid range of values acts favourably on the consolidation of the coating layer further reducing the particle release, without however having a significant effect on the surface regularity of the coating layer and without inducing significant changes in the average values of the force of static friction and of dynamic sliding friction of the plunger in the cylinder.

In particular, the Applicant has experimentally observed that the particle release values obtained with an irradiation treatment according to this preferred embodiment of the invention are significantly lower when compared to coatings that use the non-crosslinked lower viscosity silicone materials of the prior art, and comparable to those of coatings subject to irradiation treatments.

Advantageously and as described in more detail below with reference to the experiments carried out by the Applicant, this characteristic of low particle release is also substantially constant over time both by storing the cylinders at room temperature or above room temperature, and by storing the cylinders at low temperature, e.g. at temperatures in the range of from −5° C. to −40° C.

This feature is particularly appreciated in the case of medical injection devices, e.g. syringes, subject to long storage periods and/or filled with pharmaceuticals that need to be stored at low temperatures.

In addition, the Applicant has experimentally found, as will be illustrated in more detail below, that an irradiation time falling within the aforesaid range of values does not have a pejorative effect on the coating percentage of the inner surface of the cylinder, which is maintained on average at least around 90%.

In a preferred embodiment, step f) of subjecting the coating layer formed on the inner surface of the cylinder to an irradiation treatment is carried out at a time distance of at least 15 minutes, preferably of from 15 to 20 minutes, after step c) of applying the heated coating composition onto the inner surface of the cylinder.

In this way, it is advantageously possible to allow the droplets of silicone material dispensed onto the inner surface of the cylinder to coalesce with each other to achieve a coverage percentage of this surface of at least 90%.

In this regard, the Applicant has observed that waiting times of less than 15 minutes would make the coverage percentage of the inner surface of the cylinder such that to cause greater undesired interactions between the injectable liquid pharmaceutical composition stored in the cylinder and its inner glass surface.

The Applicant also noted that waiting times of more than 20 minutes did not lead to significant improvements against a significant increase in production times.

In a preferred embodiment, the manufacturing method of the invention may further comprise, before step c) of applying the heated coating composition onto the inner surface of the cylinder, a step g) of subjecting the inner surface of the cylinder to a pre-treatment to improve adhesion of the coating layer to the inner surface.

In a particularly preferred embodiment, this pre-treatment comprises forming on the inner surface of the cylinder a layer of an adhesion promoter, preferably a layer of an adhesion promoter comprising [(bicycloheptenyl)ethyl]trimethoxysilane.

Preferably, the aforesaid pre-treatment is carried out by means of the steps of:
g1) nebulizing onto the inner surface of the cylinder a solution, preferably a 2.2% by weight solution, of [(bicycloheptenyl)ethyl]trimethoxysilane in isopropyl alcohol, preferably by means of an ultrasonic static nozzle; and
g2) heating the cylinder thus treated, preferably in an oven, until the isopropyl alcohol present on the surface of the glass evaporates and thermal energy for the formation of the chemical bond between the glass and the adhesion promoter layer is provided.

In an alternative preferred embodiment, the aforesaid pre-treatment can be carried out by means of the steps of:
g1′) heating the cylinder, preferably in an oven, to a predetermined temperature; and
g2′) nebulizing onto the inner surface of the heated cylinder a solution, preferably a 2.2% by weight solution, of [(bicycloheptenyl)ethyl]trimethoxysilane in isopropyl alcohol, preferably by means of an ultrasonic static nozzle.

In this case, the cylinder is heated to a temperature suitable to subsequently evaporate the isopropyl alcohol of the nebulized solution and to provide sufficient thermal energy for the formation of the chemical bond between the glass and the layer of the adhesion promoter.

Preferably, the steps g2) and g1′) of heating the cylinder are carried out in an oven heated to a temperature preferably of from 120° C. to 145° C., more preferably, equal to about 140° C. for a time of from 14 to 25 minutes, more preferably, equal to about 20 minutes.

Preferably, the amount of the solution of [(bicycloheptenyl)ethyl]trimethoxysilane in isopropyl alcohol sprayed onto the inner surface of the cylinder is of from 7 to 50 μL, more preferably of from 7 to 22 μL.

In a preferred embodiment of the invention, the average value of the normalised concentration of the particles, released in a test solution from the coating layer of the inner surface of the cylinder, and having an average diameter equal to or greater than 10 μm or equal to or greater than 25 μm, determined by means of the LO (Light Obscuration) method according to US standard USP 787 as described in US Pharmacopeia 44-NF39 (2021), after a 3-month storage at a temperature of −40° C., is equal to or less than 60% of the limit value according to said standard.

In particular, for particles having an average diameter equal to or greater than 25 μm, this average value is equal to or less than 5% of the limit value according to said standard.

In a preferred embodiment, the average value of the normalised concentration of the particles, released in a test solution from a partially cross-linked coating layer, for example by means of an irradiation treatment, preferably by means of a plasma irradiation treatment, of the inner surface of the cylinder, and having an average diameter equal to or greater than 10 μm or equal to or greater than 25 μm, determined by means of the LO (Light Obscuration) method according to US standard USP 787 as described in US Pharmacopeia 44-NF39 (2021), after a 3-month storage at a temperature of −40° C., is equal to or less than 10% of the limit value according to said standard.

In particular, for particles having an average diameter equal to or greater than 25 μm, this average value is equal to or less than 1% of the limit value according to said standard.

Both these preferred embodiments are particularly advantageous in the case of injectable pharmaceutical compositions containing temperature-sensitive active ingredients, for example the so-called biotechnological drugs containing recombinant proteins or mRNA vaccines. These preferred embodiments, in fact, allow to achieve a significant reduction in the amount of particles released into the pharmaceutical composition stored in the cylinder of the medical injection device even after storage for a prolonged period of time at low temperature as required for the pharmaceutical compositions of this type.

In a preferred embodiment, the average value of the normalised concentration of the particles, released in a test solution from a partially cross-linked coating layer, for example by means of an irradiation treatment, preferably by means of a plasma irradiation treatment, of the inner surface of the cylinder, and having an average diameter equal to or greater than 10 μm or equal to or greater than 25 μm, determined by means of the LO (Light Obscuration) method according to US standard USP 789 as described in US Pharmacopeia 44-NF39 (2021), after a 3-month storage at a temperature of +5° C. or +25° C. or +40° C., is equal to or lower than the limit value according to said standard.

This preferred embodiment is particularly advantageous in the case of injectable pharmaceutical compositions used in the ophthalmic field for which the US standard USP 789 provides very stringent limits in relation to the maximum amount of tolerable particles in the pharmaceutical composition stored in the cylinder of the medical injection device even after storage for a prolonged period of time at the storage temperatures required for the pharmaceutical compositions of this type.

In relation to what is illustrated above, within the framework of the description and of the subsequent claims, the term “normalised” refers to normalised values with respect to the limit value of the standard considered or to the maximum value of the particle count.

In a preferred embodiment, the method of the invention further comprises a step h) of filling the cylinder of the medical injection device with an injectable liquid pharmaceutical composition, said step h) being carried out after cooling the coating layer formed on the inner surface of the cylinder to room temperature.

In this way, it is advantageously possible to obtain medical devices, e.g. syringes, pre-filled with a dosed amount of an injectable liquid pharmaceutical composition and ready for use.

In preferred embodiments of the medical injection device according to the present invention, in each arbitrary portion of the cylinder, having an axial length of 1.0 mm, and developed in plane, the coverage percentage, defined as the ratio between an area covered by the coating layer and the total measurement area, corresponding to the total area of said portion, is equal to at least 90%.

In this way, it is advantageously possible to have:
a reduced risk of an undesired contact between an injectable liquid pharmaceutical composition stored in the cylinder of the injection device and the inner glass surface of the cylinder;
optimal sliding properties (in terms of static and dynamic friction) of the plunger within the cylinder of the injection device; and
optimal properties of surface regularity of the coating layer, such as to substantially reduce the problems related to false defects that can be erroneously detected by the visual inspection devices of the medical injection device.

In preferred embodiments of the medical injection device according to the present invention, the average value of at least 30 measurements of the static sliding friction force of the plunger in the cylinder, measured on an empty cylinder of nominal volume of 1 mL at room temperature, is of from 2N to 3N.

In preferred embodiments of the medical injection device according to the present invention, the average value of at least 30 measurements of the static sliding friction force of the plunger in the cylinder, measured at room temperature on an empty cylinder of nominal volume of 0.5 mL after a 3-month storage at room temperature is of from 1N to 3N.

In preferred embodiments of the medical injection device according to the present invention, the average value of at least 30 measurements of the static sliding friction force of the plunger in the cylinder, measured on an empty cylinder of nominal volume of 1 mL after a 7-day storage at −40 C, is of from 1.5N to 3N.

In preferred embodiments of the medical injection device according to the present invention, the average value of at least 30 measurements of the dynamic sliding friction force of the plunger in the cylinder, measured on an empty cylinder of nominal volume of 1 mL at room temperature, is of from 1.5 N to 2.5 N.

In preferred embodiments of the medical injection device according to the present invention, the average value of at least 30 measurements of the dynamic sliding friction force of the plunger in the cylinder, measured at room temperature on an empty cylinder of nominal volume of 0.5 mL after a 3-month storage at room temperature is of from 1N to 2N.

In preferred embodiments of the medical injection device according to the present invention, the average value of at least 30 measurements of the dynamic sliding friction force of the plunger in the cylinder, measured on an empty cylinder of nominal volume of 1 mL after a 7-day storage at −40 C, is of from 1.5 N to 2.5 N.

Advantageously, the above-mentioned average values of the static and dynamic sliding friction force of the plunger in the cylinder are completely in line with those required by the pharmaceutical and cosmetic industry, generally 2-6N for the static sliding friction force and 1-3N for the dynamic sliding friction force. Preferably, average values of the static and dynamic sliding friction force of the plunger in the cylinder are measured by means of the following test method.

A plunger is mounted in an empty cylinder of nominal volume 1 mL Long or 0.5 mL and, within 24 h since its positioning, starting from a zero preload, a constant sliding speed is applied to the plunger equal to 240 mm/min for the cylinder of nominal volume 1 mL Long and equal to 100 mm/min for the cylinder of nominal volume 0.5 mL adapted to maintain the plunger in motion and measure by means of a dynamometer firstly the static friction force and then the dynamic friction force of the same plunger during sliding.

Additional details on this test method will be provided below in the Examples.

In preferred embodiments and as explained above in relation to the manufacturing method, the medical injection device according to the present invention comprises a partially cross-linked coating layer of the inner surface of the cylinder, preferably by means of an irradiation treatment and even more preferably by means of a plasma irradiation treatment as described above.

In preferred embodiments and as explained above in relation to the manufacturing method, the medical injection device according to the present invention may further comprise a layer of an adhesion promoter, preferably a layer of an adhesion promoter comprising [(bicycloheptenyl)ethyl]trimethoxysilane, applied to the inner surface of the cylinder.

In preferred embodiments and as explained above in relation to the manufacturing method, the medical injection device according to the present invention further comprises a plunger mounted in, and in sliding engagement with, the cylinder.

In preferred embodiments and as explained above in relation to the manufacturing method, the medical injection device according to the present invention may further comprise an injectable liquid pharmaceutical composition within the cylinder and in contact with the inner surface thereof.

In preferred embodiments, the injectable liquid pharmaceutical composition comprises a drug and/or an active ingredient in a form suitable for injection that is selected from one or more of: allergen-specific immunotherapy compositions, oligonucleotides, in particular antisense oligonucleotides and RNAi antisense oligonucleotides, biological response modifiers, blood derivatives, enzymes, monoclonal antibodies, in particular conjugated monoclonal antibodies and bispecific monoclonal antibodies, oncolytic viruses, peptides, in particular recombinant peptides and synthetic peptides, polysaccharides, proteins, in particular recombinant proteins and fusion proteins, vaccines, in particular conjugate vaccines, DNA vaccines, inactivated vaccines, mRNA vaccines, recombinant vector vaccines, subunit vaccines, or combinations thereof insofar compatible.

More preferably, said drug and/or active ingredient in a form suitable for injection is selected from: GEN-3009, (human insulin analogue A21G+pramlintide), (AZD-5069+durvalumab), (futuximab+modotuximab), [225Ac]-FPI-1434, 111 In-CP04, 14-F7, 212 Pb-TCMC-Trastuzumab, 2141 V-11, 3BNC-117LS, 3K3A-APC, 8H-9, 9MW-0211, A-166, A-319, AADvac-1, AB-002, AB-011, AB-022, AB-023, AB-154, AB-16B5, AB-729, ABBV-011, ABBV-0805, ABBV-085, ABBV-151, ABBV-154, ABBV-155, ABBV-184, ABBV-3373, ABBV-368, ABBV-927, abelacimab, AbGn-107, AbGn-168H, ABL-001, ABvac-40, ABY-035, acetylcysteine+bromelain, ACI-24, ACI-35, ACP-014, ACP-015, ACT-101, Actimab-A, Actimab-M, AD-214, adavosertib+durvalumab, ADCT-602, ADG-106, ADG-116, ADM-03820, AdVince, AEX-6003, aflibercept biosimilar, AFM-13, AGEN-1181, AGEN-2373, AGLE-177, AGT-181, AIC-649, AIMab-7195, AK-101, AK-102, AK-104, AK-109, AK-111, AK-112, AK-119, AK-120, AL-002, AL-003, AL-101, aldafermin, aldesleukin, ALG-010133, ALM-201, ALMB-0168, ALNAAT-02, ALNAGT-01, ALN-HSD, ALPN-101, ALT-801, ALTP-1, ALTP-7, ALX-0141, ALX-148, ALXN-1720, AM-101, amatuximab, AMC-303, amelimumab, AMG-160, AMG-199, AMG-224, AMG-256, AMG-301, AMG-330, AMG-404, AMG-420, AMG-427, AMG-509, AMG-673, AMG-701, AMG-714, AMG-757, AMG-820, AMRS-001, AMV-564, AMY-109, AMZ-002, Analgecine, Ancrod, Andecaliximab, Anetumab corixetan, Anetumab ravtansine, ANK-700, Antibodies for snake poisoning, Antibody for anthrax, Antibody for Coronavirus Disease 2019 (COVID-19), Antibody for tetanus, Antibody for type 1 diabetes, Antibody for OX40 agonist for solid tumours, antihaemophilic factor (recombinant), Antisense Oligonucleotide RNAi to inhibit EPHA2 for solid tumours and ovarian cancer, ANX-007, ANX-009, AP-101, Apitegromab, APL-501, APL-501, APN-01, APS-001+flucytosine, APSA-01, APT-102, APVAC-1, APVAC-2, APVO-436, APX-003, APX-005M, ARCT-810, ARGX-109, ARGX-117, AROANG-3, AROAPOC-3, AROHIF-2, ARO-HSD, Ascrinvacumab, ASLAN-004, ASP-1235, ASP-1650, ASP-9801, AST-008, Astegolimab, Asunercept, AT-1501, Atacicept, ATI-355, ATL-101, ATOR-1015, ATOR-1017, ATP-128, ATRC-101, Atrosab, ATX-101, ATXGD-59, ATXMS-1467, ATYR-1923, AU-011, Rituximab (coniugated) (Aurixim R), AV-1, AVB-500, Avdoralimab, AVE-1642, AVI-3207, AVID-100, AVID-200, Aviscumine, Avizakimab, Axatilimab, B-001, B-002, Barusiban, BAT-1306, BAT-4306, BAT-4406F, BAT-5906, BAT-8003, batroxobin, BAY-1905254, BAY-2315497, BAY-2701439, BB-1701, BBT-015, BCD-096, BCD-131, BCD-217, BCT-100, Bemarituzumab, Bepranemab, Bermekimab, Bertilimumab, Betalutin, Bevacizumab, Bexmarilimab, BG-00010, BGBA-445, BHQ-880, BI-1206, BI-1361849, BI-456906, BI-655064, BI-655088, BI-754091, BI-754111, BI-836858, BI-836880, BI-905677, BI-905711, BIIB-059, BIIB-076, BIIB-101, BIL-06v, Bimagrumab, BIO89-100, Biological response modifier for Coronavirus disease 2019 (COVID-19), Urinary tract infections, prosthetic joint and Acinetobacter infections, Biological response modifier for unspecified indication, Bispecific monoclonal antibody 1 for diabetic macular oedema and wet macular degeneration, Bispecific monoclonal antibody to inhibit HIV 1 Env for HIV infections, Bispecific monoclonal antibody to detect GD2 and CD3 for oncology, Bispecific monoclonal antibody to detect PD-L1 and CTLA4 for pancreatic duct adenocarcinoma, BIVV-020, Bleselumab, BM-32, BMS-986012, BMS-986148, BMS-986156, BMS-986178, BMS-986179, BMS-986207, BMS-986218, BMS-986226, BMS-986253, BMS-986258, BMS-986258, BMS-986263, BNC-101, BNT-111, BNT-112, BNT-113, BNT-114, BNT-121, BOS-580, Botulinum toxin, BP-1002, BPI-3016, BrevaRex MAb-AR20.5, Brivoligide, Bromelain, BT-063, BT-1718, BT-200, BT-5528, BT-588, BT-8009, BTI-322, BTRC-4017A, Budigalimab, BXQ-350, CI esterase inhibitors (human), cabiralizumab, camidanlumab tesirine, canerpaturev, Cavatak, CBA-1205, CBP-201, CBP-501, CC-1, CC-90002, CC-90006, CC-93269, CC-99712, CCW-702, CDX-0159, CDX-301, CDX-527, Celyvir, cemdisiran, cendakimab, CERC-002, CERC-007, cevostamab, cibisatamab, CIGB-128, CIGB-258, CIGB-300, CIGB-500, CIGB-552, CIGB-814, CIGB-845, cinpanemab, cinrebafusp alfa, CIS-43, CiVi-007, CJM-112, CKD-702, Clustoid D. pteronyssinus, CM-310, CMK-389, CMP-001, CNTO-6785, CNTO-6785, CNV-NT, coagulation factor VIII (recombinant), cobomarsen, codrituzumab, cofetuzumab pelidotin, COR-001, cosibelimab, cosibelimab, cotadutide, CPI-006, CRX-100, CSJ-137, CSL-311, CSL-324, CSL-346, CSL-730, CSL-889, CTB-006, CTI-1601, CTP-27, CTX-471, CUE-101, cusatuzumab, CV-301, CVBT-141, CX-2009, CX-2029, CYN-102, CyPep-1, CYT-107, CYT-6091, anti-cytomegalovirus immune globulin (human), dabrafenib mesylate+panitumumab+trametinib dimethyl sulfoxide, DAC-002, dalcinonacog alfa, dalotuzumab, danvatirsen+durvalumab, dapiglutide, daxdilimab, DB-001, DCRA-1AT, Dekavil, depatuxizumab, desmopressin, DF-1001, DF-6002, Diamyd, dilpacimab, diridavumab, DK-001, DKN-01, DM-101, DM-199, DMX-101, DNL-310, DNP-001, DNX-2440, domagrozumab, donanemab, donidalorsen sodium, DP-303c, DS-1055a, DS-2741, DS-6157, DS-7300, DS-8273, durvalumab+monalizumab, durvalumab+oleclumab, durvalumab+oportuzumab monatox, durvalumab+selumetinib sulphate, DX-126262, DXP-593, DXP-604, DZIF-10c, E-2814, E-3112, EBI-031, Yttrium-90 labelled Edotreotide efavaleukin alfa, efpegsomatropin, efruxifermin, eftilagimod alfa, eftozanermin alfa, EG-Mirotin, elezanumab, elipovimab, emactuzumab, enadenotucirev, Engedi-1000, ensituximab, EO-2401, epcoritamab, ERY-974, etigilimab, etokimab, Evitar, EVX-02, Exenatide, F-0002ADC, F-520, F-598, F-652, faricimab, FAZ-053, FB-704A, FB-825, FF-21101, fibrinogen concentrate (human), ficlatuzumab, flotetuzumab, FLYSYN, FmAb-2, FNS-007, FOL-005, FOR-46, foralumab, Foxy-5, FPP-003, FR-104, fresolimumab, FS-102, FS-118, FS-120, FS-1502, FSH-GEX, Fusion protein for allergic asthma, Fusion protein to antagonize thrombopoietin receptor for idiopathic thrombocytopenia purpura, Fusion protein to antagonize EGFR for glioblastoma multiforme and malignant glioma, Fusion protein to inhibit CD25 for oncology, Fusion protein to target mesothelin for oncology, Fusion proteins for colitis, hypertension and ulcerative colitis, FX-06, G-035201, G-207, G-3215, garetosmab, gatipotuzumab, GB-223, GBB-101, GC-1118A, GC-5131A, GEM-103, GEM-333, GEM-3PSCA, gemibotulinumtoxin A, GEN-0101, GEN-1046, Gensci-048, gentuximab, gevokizumab, glenzocimab, glofitamab, glucagon, GM-101, GMA-102, GMA-301, GNR-051, GNR-055, GNR-084, GNX-102, goserelin acetate, gosuranemab, gp-ASIT, GR-007, GR-1401, GR-1405, GR-1501, GRF-6019, GRF-6021, GS-1423, GS-2872, GS-5423, GSK-1070806, GSK-2241658A, GSK-2330811, GSK-2831781, GSK-3174998, GSK-3511294, GSK-3537142, GT-02037-, GT-103, GTX-102, GW-003, GWN-323, GX-301, GXG-3, GXP-1, H-11B6, HAB-21, HALMPE-1, HB-0021, HBM-4003, HDIT-101, HER-902, HFB-30132A, HH-003, HL-06, HLX-06, HLX-07, HLX-20, HLX-22, HM-15211, HM-15912, HM-3, HPN-217, HPN-328, HPN-424, HPN-536, HPV-19, hRESCAP, HS-214, HS-628, HS-630, HS-636, HSV-1716, HTD-4010, HTI-1066, Hu8F4, HUB-1023, hVEGF-26104, HX-009, Hyaluronidase (recombinant), IBI-101, IBI-110, IBI-112, IBI-188, IBI-302, IBI-318, IBI-322, IBI-939, IC-14, ICON-1, ICT-01, ieramilimab, ifabotuzumab, IGEM-F, IGM-2323, IGM-8444, IGN-002, IMA-950, IMA-970A, IMC-002, IMCF-106C, IMCY-0098, IMGN-632, IMM-005, IMM-01, IMM-201, immunoglobulin (human), imsidolimab, INA-03, INBRX-101, INBRX-105, INBRX-105, INCAGN-1876, INCAGN-1949, INCAGN-2385, inclacumab, indatuximab ravtansine, interferon alfa-2b, interferon alfa-2b, INVAC-1, IO-102, IO-103, IO-112, IO-202, ION-224, ION-251, ION-464, ION-537, ION-541, ION-859, IONIS-AGTLRx, IONISAR-2.5Rx, IONIS-C9Rx, IONIS-FB-LRx, IONIS-FXILRx, IONIS-FXIRx, IONIS-GCGRRx, IONIS-HBVLRx, IONIS-HBVRx, IONIS-MAPTRx, IONIS-PKKRx, IONISTMPRSS-6LRx, IPN-59011, IPP-204106, Ir-CPI, IRL-201104, IRL-201805, ISA-101, ISB-1302, ISB-1342, ISB-830, iscalimab, ISU-104, IT-1208, ITF-2984, IXTM-200, JBH-492, JK-07, JMT-101, JMT-103, JNJ-0839, JNJ-3657, JNJ-3989, JNJ-4500, JNJ-67571244, JNJ-75348780, JNJ-9178, JS-003, JS-004, JS-005, JSP-191, JTX-4014, JY-025, JZB-30, JZB-34, K-170, K-193, KAN-101, KD-033, KER-050, KH-903, KHK-4083, KHK-6640, EDV paediatric, KLA-167, KLA-167, KLT-1101, KMRC-011, KN-026, KPL-404, KSI-301, KTN-0216, KTP-001, KUR-113, KY-1005, KY-1044, labctuzumab govitecan, lacnotuzumab, lacutamab, ladiratuzumab vedotin, laronidase, LBL-007, LDOS-47, letolizumab, leuprolide acetate, LEVI-04, LH-021, liatermine, lirilumab, LIS-1, LKA-651, LLF-580, LMB-100, LNA-043, LOAd-703, lodapolimab, lorukafusp alfa, LP-002, LT-1001, LT-1001, LT-1001, LT-3001, LT-3001, LTI-01, LTX-315, LuAF-82422, LuAF-87908, lulizumab pegol, LVGN-6051, LY-3016859, LY-3022855, LY-3041658, LY-3305677, LY-3372993, LY-3375880, LY-3434172, LY-3454738, LY-3561774, LZM-009, M-032, M-1095, M-254, M-6495, M-701, M-802, M-9241, MAG-Tn3, MAU-868, MB-108, MBS-301, MCLA-117, MCLA-145, MCLA-158, MDNA-55, MDX-1097, MEDI-0457, MEDI-0618, MEDI-1191, MEDI-1341, MEDI-1814, MEDI-3506, MEDI-3617+tremelimumab, MEDI-5117, MEDI-5395, MEDI-570, MEDI-5752, MEDI-5884, MEDI-6012, MEDI-6570, MEDI-7352, MEDI-9090, MEN-1112, meplazumab, mezagitamab, MG-021, MG-1113A, MGC-018, MIL-62, MIL-77, MIL-86, mitazalimab, MK-1654, MK-3655, MK-4166, MK-4280, MK-4621, MK-5890, Molgramostim, Conjugated monoclonal antibody to identify CD276 for oncology, Conjugated monoclonal antibody to identify CD45 for oncology, Conjugated monoclonal antibody to identify CEACAM5 for non-small cell lung cancer and metastatic colorectal cancer, Conjugated monoclonal antibody to identify Mucin 1 for metastatic colorectal cancer, Conjugated monoclonal antibody to target PSMA for prostate cancer, Monoclonal antibody for Dengue, Monoclonal antibody to antagonize IL-2R Beta for celiac disease, oncology and tropical spastic paraparesis, Monoclonal antibody to antagonize Interleukin-6 receptor for rheumatoid arthritis, Monoclonal antibody to antagonize PD1 for oncology, Monoclonal antibody to antagonize PD1 for solid tumours, Monoclonal antibody to inhibit CD4 for HIV-1, Monoclonal antibody to inhibit GD2 for oncology, Monoclonal antibody to inhibit glycoprotein for rabies, Monoclonal antibody to inhibit IL17 for autoimmune and musculoskeletal disorders, Monoclonal antibody to inhibit IL5 for asthma and chronic obstructive pulmonary disease (COPD), Monoclonal antibody to inhibit PD-L1 for solid tumours, Monoclonal antibody to inhibit TNF-alfa for ankylosing spondylitis, psoriasis and rheumatoid arthritis, Monoclonal antibody to inhibit TNF-Alfa for Dupuytren's contracture, Monoclonal antibody to inhibit VEGF for diabetic macular oedema and wet age-related macular degeneration, Monoclonal antibody to inhibit VEGF for oncology and ophthalmology, Monoclonal antibody to inhibit VEGFA for metastatic colorectal cancer and non-small cell lung cancer, Monoclonal antibody to target CD66b for blood cancer and metabolic disorders, Monoclonal antibody to target GP41 for HIV infections, MORAb-202, Motrem, MP-0250, MP-0274, MP-0310, MP-0420, MRG-001, MRG-002, MRG-003, MRG-110, mRNA-2416, mRNA-2752, mRNA-3927, MSB-0254, MSB-2311, MSC-1, MT-1001, MT-1002, MT-2990, MT-3724, MT-3921, MTX-102, murlentamab, MVT-5873, MVXONCO-1, MW-11, MW-33, NA-704, namilumab, naratuximab emtansine, navicixizumab, NBE-002, NBF-006, NC-318, NC-410, nemvaleukin alfa, NEOPV-01, NG-348, NG-350a, NG-641, NGM-120, NGM-395, NGM-621, NI-006, NI-0801, nidanilimab, nimacimab, NIS-793, NIZ-985, NJA-730, NJH-395, NKTR-255, NKTR-358, NMIL-121, NN-9215, NN-9499, NN-9775, NN-9838, NN-9931, NNC-03850434, NP-024, NP-025, NP-137, NPC-21, NPT-088, NPT-189, NRP-2945, NStride APS, NVG-111, NXT-007, NZV-930, OBI-888, OBI-999, OBT-076, OC-001, octreotide acetate, octreotide acetate CR, octreotide acetate microspheres, odronextamab, odronextamab, OH-2, olamkicept, oleclumab, olinvacimab, olpasiran, olvimulogene nanivacirepvec, OMS-906, onabotulinumtoxin A, ONC-392, ONCase-PEG, Oncolytic virus for Human papillomavirus-associated cancer, Human papillomavirus infections and Coronavirus disease 2019 (COVID-19), Oncolytic virus for metastatic breast cancer, Oncolytic virus for oncology, Oncolytic virus for solid tumour, Oncolytic virus to activate IL-12 for recurrent prostate cancer and metastatic pancreatic cancer, Oncolytic virus to activate thymidine kinase for oncology, Oncolytic virus to antagonize PD1 for solid tumours, Oncolytic virus to target CD155/NECL5 for solid tumours, Oncolytic virus to target CD46 and SLC5A5 for oncology, Oncolytic virus to target E6 and E7 for Human papillomavirus (HPV)-associated solid tumours, Oncolytic virus to target MAGE-A3 for solid tumours, ONCOS-102, ONCR-177, ongericimab, ONO-4685, onvatilimab, OPK-88005, OPT-302, ORCA-010, OrienX-010, orilanolimab, orticumab, OS-2966, OSE-127, osocimab, otelixizumab, OTO-413, OTSA-101, OXS-1550, OXS-3550, P-28R, P-2G12, pacmilimab, panobacumab, Parvoryx, pasireotide, pasotuxizumab, PC-mAb, PD-01, PD-0360324, PD-1+antagonist ropeginterferon alfa-2b, pegbelfermin, peginterferon lambda-la, pelareorep, pelareorep, Pemziviptadil, PEN-221, pentosan sodium polysulfate, pepinemab, pepinemab, Peptide for Coronavirus Disease 2019 (COVID-19), Peptide for solid tumours, pertuzumab biobetter, pexastimogene devacirepvec, PF-04518600, PF-06480605, PF-06730512, PF-06755347, PF-06804103, PF-06817024, PF-06823859, PF-06835375, PF-06863135, PF-06940434, PF-07209326, PF-655, PHN-013, PHN-014, PHN-015, pidilizumab, PIN-2, plamotamab, plasminogen (human) 1, Plexaris, PM-8001, PNT-001, Pollinex Quattro Tree, PolyCAb, Poly-ICLC, PolyPEPI-1018, ponsegromab, PP-1420, PR-15, PR-200, prasinezumab, prexigebersen, PRL3-ZUMAB, Protein for diabetic foot ulcers and brain haemorrhage, protein for osteoarthritis and asthma, protein to activate IL12 for infectious diseases and oncology, PRS-060, PRTX-100, PRV-300, PRV-3279, PRX-004, PSB-205, PT-101, PT-320, PTR-01, PTX-35, PTX-9908, PTX-9908, PTZ-329, PTZ-522, PVX-108, QBECO-SSI, QBKPN-SSI, QL-1105, QL-1203, QL-1207, QL-1604, QPI-1007, QPI-1007, quavonlimab, quetmolimab, QX-002N, QX-005N, Radspherin, ranibizumab, ranpirnase, ravagalimab, next generation ravulizumab, RC-28, RC-402, RC-88, RD-001, REC-0438, Recombinant carboxypeptidase G2 for methotrexate toxicity, recombinant enzyme for organophosphorus nerve agent poisoning, recombinant peptide to agonize GHRH for cardiovascular, central nervous system, musculoskeletal and metabolic disorders, recombinant plasma Gelsolin substitute for infectious diseases, Recombinant protein for inflammatory bowel diseases, multiple sclerosis and psoriasis, Recombinant protein for oncology, Recombinant protein to agonize IFNAR1 and IFNAR2 for oncology, Recombinant protein to agonize KGFR for chemotherapy-induced gastrointestinal mucositis and oral mucositis, Recombinant protein to agonize thrombopoietin receptor for idiopathic thrombocytopenia purpura. Recombinant protein to inhibit CD13 for lymphoma and solid tumour, recombinant protein to inhibit coagulation factor XIV for haemophilia A and haemophilia B, recombinant urate oxidase substitute for acute hyperuricemia, redasemtide trifluoroacetate, REGN-1908 1909, REGN-3048, REGN-3051, REGN-3500, REGN-4018, REGN-4461, REGN-5093, REGN-5458, REGN-5459, REGN-5678, REGN-5713, REGN-5714, REGN-5715, REGN-6569, REGN-7075, REGN-7257, remlarsen, Renaparin, REP-2139, REP-2165, reteplase, RG-6139, RG-6147, RG-6173, RG-6290, RG-6292, RG-6346, RG-70240, RG-70240, RG-7826, RG-7835, RG-7861, RG-7880, RG-7992, RGLS-4326, Rigvir, rilimogene galvacirepvec, risuteganib, rituximab, RMC-035, RO-7121661, RO-7227166, RO-7284755, RO-7293583, RO-7297089, romilkimab, ropocamptide, rozibafusp alfa, RPH-203, RPV-001, rQNestin-34.5v.2, RSLV-132, RV-001, RXI-109, RZ-358, SAB-176, SAB-185, SAB-301, SAIT-301, SAL-003, SAL-015, SAL-016, Sanguinate, SAR-439459, SAR-440234, SAR-440894, SAR-441236, SAR-441344, SAR-442085, SAR-442257, SB-11285, SBT-6050, SCB-313, SCIB-1, SCO-094, SCT-200, SCTA-01, SD-101, SEA-BCMA, SEA-CD40, SelectAte, selicrelumab, SelK-2, semorinemab, serclutamab talirine, seribantumab, setrusumab, sodium sevuparin, SFR-1882, SFR-9213, SFR-9216, SFR-9314, SG-001, SGNB-6A, SGNCD-228A, SGN-TGT, SHR-1209, SHR-1222, SHR-1501, SHR-1603, SHR-1701, SHR-1702, SHR-1802, SHRA-1201, SHRA-1811, SIB-001, SIB-003, simlukafusp alfa, siplizumab, sirukumab, SKB-264, SL-172154, SL-279252, SL-701, SOC-101, SOJB, somatropin SR, sotatercept, sprifermin, SRF-617, SRP-5051, SSS-06, SSS-07, ST-266, STA-551, STI-1499, STI-6129, STK-001, STP-705, STR-324, STRO-001, STRO-002, STT-5058, SubQ-8, sulituzumab, suvratoxumab, SVV-001, SY-005, SYD-1875, Sym-015, Sym-021, Sym-022, Sym-023, SYN-004, SYN-125, Synthetic peptide to inhibit SLC10A1 for hepatitis B and type 2 diabetes, synthetic peptide to modulate GHSR for chronic kidney disease, synthetic peptide to target CCKBR for medullary thyroid cancer, synthetic peptide to target somatostatin receptor for neuroendocrine gastroenteropancreatic tumours, T-3011, T-3011, TA-46, TAB-014, TAB-014, sodium tafoxiparin, TAK-101, TAK-169, TAK-573, TAK-611, TAK-671, talquetamab, tasadenoturev, TBio-6517, TBX.OncV NSC, tebotelimab, teclistamab, telisotuzumab vedotin, telomelysin, temelimab, tenecteplase, tesidolumab, teverelix, TF-2, TG-1801, TG-4050, TG-6002, TG-6002, T-Guard, THOR-707, THR-149, THR-317, Thrombosomes, Thymalfasin, tilavonemab, TILT-123, tilvestamab, tinurilimab, tipapkinogene sovacivec, tiprelestat, TM-123, TMB-365, TNB-383B, TNM-002, TNX-1300, tomaralimab, tomuzotuximab, tonabacase, tralesinidase alfa, trebananib, trevogrumab, TRK-950, TRPH-222, TRS-005, TST-001, TTHX-1114, TTI-621, TTI-622, TTX-030, TVT-058, TX-250, TY-101, tyzivumab, U-31402, UB-221, UB-311, UB-421, UB-621, UBP-1213, UC-961, UCB-6114, UCHT-1, UCPVax, ulocuplumab, UNEX-42, UNI-EPO-Fc, urelumab, UV-1, V-938, Vaccine for acute lymphocytic leukaemia, Vaccine for B-cell non-Hodgkin's lymphoma, Vaccine for chronic lymphocytic leukaemia, Vaccine for glioma, Vaccine for hormone-sensitive prostate cancer, Vaccine for melanoma, Vaccine for non-muscle invasive bladder cancer, Vaccine for ovarian cancer, Vaccine to target Brachyury and HER2 for oncology, Vaccine to target Brachyury for oncology, Vaccine to target CCL20 for B-cell non-Hodgkin's lymphomas, Vaccine to target CEA for colorectal cancer, Vaccine to target IFN-Alfa for metabolic disorders, immunology, infectious diseases and musculoskeletal disorders, VAL-201, vantictumab, vanucizumab, varlilumab, Vas-01, VAX-014, VB-10NEO, VCN-01, vibecotamab, vibostolimab, VIR-2218, VIR-2482, VIR-3434, VIS-410, VIS-649, vixarelimab, VLS-101, vofatamab, volagidemab, vopratelimab, Voyager-V1, VRC-01, VRC-01LS, VRC-07523LS, VTP-800, vunakizumab, vupanorsen sodium, Vx-001, Vx-006, W-0101, WBP-3425, XAV-19, xentuzumab, XmAb-20717, XmAb-22841, XmAb-23104, XmAb-24306, XMT-1536, XoGlo, XOMA-213, XW-003, Y-14, Y-242, YH-003, YH-14618, YS-110, YYB-101, zagotenemab, zalifrelimab, zampilimab, zanidatamab, zanidatamab, zansecimab, zenocutuzumab, ZG-001, ZK-001, ZL-1201, Zofin, or combinations thereof insofar compatible.

In preferred embodiments, the kit of parts for assembling a medical injection device according to the invention, comprises the preferred features of the medical device described above as far as applicable.

These and other aspects are merely illustrative of the innumerable aspects associated with the present disclosure and should not be deemed as limiting in any manner. These and other aspects, features, and advantages of the present disclosure will become apparent from the following detailed description when taken in conjunction with the referenced drawings.

### BRIEF DESCRIPTION OF THE DRAWINGS

Reference is now made more particularly to the drawings, which illustrate the best presently known mode of carrying out the present disclosure and wherein similar reference characters indicate the same parts throughout the views.

FIG. 1 illustrates in partial longitudinal section a medical injection device, in particular a syringe, according to a preferred embodiment of the invention;

FIG. 2 shows a block diagram schematically illustrating an apparatus for manufacturing a medical injection device according to a preferred embodiment of the invention;

FIGS. 3 and 4 show as many graphs illustrating the profile along the axial development of a cylinder of a medical injection device of nominal volume of 1 mL and, respectively, of 3 mL, of the thickness of an exemplary coating layer applied to the inner surface of the cylinder, according to a preferred embodiment of the invention;

FIGS. 5-10 show as many graphs illustrating the profile along the axial development of a cylinder of a medical injection device of nominal volume of 0.5 mL, of the thickness, measured at room temperature immediately after the application and cooling of the coating layer (t0) and after a 3-month storage (t3) at room temperature, of an exemplary coating layer applied to the inner surface of the cylinder according to preferred embodiments of the invention and according to the prior art;

FIG. 11 shows the average values of the static sliding friction force of a plunger mounted in an empty cylinder having a nominal volume of 1 mL of some examples of medical injection devices according to the invention and according to the prior art at different time points;

FIG. 12 shows the average values of the dynamic sliding friction force of a plunger mounted in an empty cylinder having a nominal volume of 1 mL of some examples of medical injection devices according to the invention and according to the prior art at different time points;

FIG. 13 shows the average values of the static sliding friction force of a plunger mounted in a cylinder having a nominal volume of 1 mL, filled with a test solution having a dynamic viscosity of 1 mPa·s, of some examples of medical injection devices according to the invention and according to the prior art at different time points;

FIG. 14 shows the average values of the dynamic sliding friction force of a plunger mounted in a cylinder having a nominal volume of 1 mL, filled with a test solution having a dynamic viscosity of 1 mPa·s, of some examples of medical injection devices according to the invention and according to the prior art at different time points;

FIG. 15 shows the average values of the static sliding friction force of a plunger mounted in a cylinder with a nominal volume of 1 mL, filled with a test solution, of some examples of medical injection devices according to the invention and according to the prior art after a 7-day storage time at different temperatures;

FIG. 16 shows the average values of the dynamic sliding friction force of a plunger mounted in a cylinder having a nominal volume of 1 mL, filled with a test solution, of some examples of medical injection devices according to the invention and according to the prior art after a 7-day storage times at different temperatures;

FIG. 17 shows the average values of the static sliding friction force of a plunger mounted in a cylinder having a nominal volume of 1 mL, filled with a test solution, of some examples of medical injection devices according to the invention and according to the prior art after a storage time of 2 and 7 days at a temperature of −40° C.;

FIG. 18 shows the average values of the dynamic sliding friction force of a plunger mounted in a cylinder having a nominal volume of 1 mL, filled with a test solution, of some examples of medical injection devices according to the invention and according to the prior art after a storage time of 2 and 7 days at a temperature of −40° C.;

FIG. 19 shows the average values of the static sliding friction force of a plunger mounted in an empty cylinder having a nominal volume of 0.5 mL measured at room temperature of some examples of medical injection devices according to the invention and according to the prior art at different time points;

FIG. 20 shows the average values of the dynamic sliding friction force of a plunger mounted in an empty cylinder having a nominal volume of 0.5 mL measured at room temperature of some examples of medical injection devices according to the invention and according to the prior art at different time points;

FIG. 21 shows the average values of the static sliding friction force of a plunger mounted in a cylinder with a nominal volume of 0.5 mL, filled with a test solution, of some examples of medical injection devices according to the invention and according to the prior art at different time points using a storage temperature of −40° C.;

FIG. 22 shows the average values of the dynamic sliding friction force of a plunger mounted in a cylinder with a nominal volume of 0.5 mL, filled with a test solution, of some examples of medical injection devices according to the invention and according to the prior art at different time points and using a storage temperature of −40° C.;

FIG. 23 shows the average values of the static sliding friction force of a plunger mounted in a cylinder with a nominal volume of 0.5 mL, filled with a test solution, of some examples of medical injection devices according to the invention and according to the prior art at different time points and using a storage temperature of +5° C.;

FIG. 24 shows the average values of the dynamic sliding friction force of a plunger mounted in a cylinder with a nominal volume of 0.5 mL, filled with a test solution, of some examples of medical injection devices according to the invention and according to the prior art at different time points and using a storage temperature of +5° C.;

FIG. 25 shows the average values of the static sliding friction force of a plunger mounted in a cylinder with a nominal volume of 0.5 mL, filled with a test solution, of some examples of medical injection devices according to the invention and according to the prior art at different time points and using a storage temperature of +25° C.;

FIG. 26 shows the average values of the dynamic sliding friction force of a plunger mounted in a cylinder with a nominal volume of 0.5 mL, filled with a test solution, of some examples of medical injection devices according to the invention and according to the prior art at different time points and using a storage temperature of +25° C.;

FIG. 27 shows the average values of the static sliding friction force of a plunger mounted in a cylinder with a nominal volume of 0.5 mL, filled with a test solution, of some examples of medical injection devices according to the invention and according to the prior art at different time points and using a storage temperature of +40° C.;

FIG. 28 shows the average values of the dynamic sliding friction force of a plunger mounted in a cylinder with a nominal volume of 0.5 mL, filled with a test solution, of some examples of medical injection devices according to the invention and according to the prior art at different time points and using a storage temperature of +40° C.;

FIG. 29 summarizes the average values of the static sliding friction force of a plunger mounted in a cylinder having a nominal volume of 0.5 mL, filled with a test solution, shown in FIGS. 21-28, of examples of medical injection devices according to the invention and according to the prior art after a three-month storage at different temperatures;

FIG. 30 summarizes the average values of the dynamic sliding friction force of a plunger mounted in a cylinder having a nominal volume of 0.5 mL, filled with a test solution, shown in FIGS. 21-28, of examples of medical injection devices according to the invention and according to the prior art after a three-month storage at different temperatures;

FIG. 31 shows the normalised values of the concentration of particles with a size equal to or greater than 10 μm of examples of medical injection devices having a cylinder with nominal filling volume of 3.0 mL, filled with 3.3 mL of an aqueous test solution and subjected to automated stirring (360° rotation of the samples), according to the invention and according to the prior art measured at room temperature:

FIG. 32 shows the normalised values of the concentration of particles with a size equal to or greater than 25 μm of examples of medical injection devices having a cylinder with nominal filling volume of 3.0 mL, filled with 3.3 mL of an aqueous test solution and subjected to automated stirring (360° rotation of the samples), according to the invention and according to the prior art measured at room temperature:

FIGS. 33-35 show the normalised values of the concentration of particles with a size equal to or greater than 10 μm measured at three different temperature conditions at a time 0 and after a storage for 6 months, of examples of medical injection devices having a cylinder with nominal filling volume of 0.5 mL, filled with 0.25 mL of an aqueous test solution, according to the invention and according to the prior art;

FIG. 36 shows the normalised values of the concentration of particles of examples of medical injection devices according to the invention and according to the prior art having a cylinder with nominal filling volume of 1.0 mL, filled with 0.55 mL of an aqueous test solution, determined by the MFI test measured at different storage times at a temperature of −40° C.;

FIGS. 37 and 38 show the normalised values of the concentration of particles with a size equal to or greater than 10 μm and, respectively, equal to or greater than 25 μm, of examples of medical injection devices according to the invention and according to the prior art having a cylinder with nominal filling volume of 0.5 mL, filled with 500 μL of an aqueous test solution, measured at a temperature of −40° C.;

FIGS. 39 and 40 show the particle release values of examples of medical injection devices according to the invention and according to the prior art having a cylinder with nominal filling volume of 0.5 mL, filled with 500 μL of an aqueous test solution, measured by the MFI test at different storage times at a temperature of −40° C.;

FIGS. 41 and 42 show the normalised values of the concentration of particles with a size equal to or greater than 10 μm and, respectively, equal to or greater than 25 μm, of examples of medical injection devices according to the invention and according to the prior art having a cylinder with nominal filling volume of 0.5 mL, filled with 500 μL of an aqueous test solution, measured at a temperature of +5° C.;

FIGS. 43 and 44 show the normalised values of the concentration of particles with a size equal to or greater than 10 μm and, respectively, equal to or greater than 25 μm, of examples of medical injection devices according to the invention and according to the prior art having a cylinder with nominal filling volume of 0.5 mL, filled with 500 μL of an aqueous test solution, measured at a temperature of +5° C. and with a coating subjected to a plasma irradiation treatment;

FIGS. 45 and 46 show the particle release values of examples of medical injection devices according to the invention and according to the prior art having a cylinder with nominal filling volume of 0.5 mL, filled with 500 μL of an aqueous test solution, measured by the MFI test at different storage times at a temperature of +5° C.;

FIGS. 47 and 48 show the normalised values of the concentration of particles with a size equal to or greater than 10 μm and, respectively, equal to or greater than 25 μm, of examples of medical injection devices according to the invention and according to the prior art having a cylinder with nominal filling volume of 0.5 mL, filled with 500 μL of an aqueous test solution, measured at a temperature of +25° C.;

FIGS. 49 and 50 show the normalised values of the concentration of particles with a size equal to or greater than 10 μm and, respectively, equal to or greater than 25 μm, of examples of medical injection devices according to the invention and according to the prior art having a cylinder with nominal filling volume of 0.5 mL, filled with 500 μL of an aqueous test solution, measured at a temperature of +25° C. and with a coating subjected to a plasma irradiation treatment;

FIGS. 51 and 52 show the particle release values of examples of medical injection devices according to the invention and according to the prior art having a cylinder with nominal filling volume of 0.5 mL, filled with 500 μL of an aqueous test solution, measured by the MFI test at different storage times at a temperature of +25° C.;

FIGS. 53 and 54 show the normalised values of the concentration of particles with a size equal to or greater than 10 μm and, respectively, equal to or greater than 25 μm, of examples of medical injection devices according to the invention and according to the prior art having a cylinder with nominal filling volume of 0.5 mL, filled with 500 μL of an aqueous test solution, measured at a temperature of +40° C.;

FIGS. 55 and 56 show the normalised values of the concentration of particles with a size equal to or greater than 10 μm and, respectively, equal to or greater than 25 μm, of examples of medical injection devices according to the invention and according to the prior art having a cylinder with nominal filling volume of 0.5 mL, filled with 500 μL of an aqueous test solution, measured at a temperature of +40° C. and with a coating subjected to a plasma irradiation treatment;

FIGS. 57 and 58 show the particle release values of examples of medical injection devices according to the invention and according to the prior art having a cylinder with nominal filling volume of 0.5 mL, filled with 500 μL of an aqueous test solution, measured by the MFI test at different storage times at a temperature of +40° C.;

FIGS. 59 and 60 summarize the normalised values of the concentration of particles with a size equal to or greater than 10 μm and, respectively, equal to or greater than 25 μm, of examples of medical injection devices according to the invention and according to the prior art having a cylinder with nominal filling volume of 0.5 mL, filled with 500 μL of an aqueous test solution, with a coating subjected to a plasma irradiation treatment, after a three-month storage at different temperatures;

FIGS. 61-67 show as many photographs made by means of an optical microscope of coating layers of a silicone material according to the invention and according to the prior art subjected to partial cross-linking by plasma irradiation at various irradiation times and in various areas of the cylinder of a medical injection device.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

In the following detailed description numerous specific details are set forth in order to provide a thorough understanding of the present disclosure. However, it will be understood by those skilled in the art that the present disclosure may be practiced without these specific details. For example, the present disclosure is not limited in scope to the particular type of industry application depicted in the figures. In other instances, well-known methods, procedures, and components have not been described in detail so as not to obscure the present disclosure.

The headings and sub-headings used herein are intended only for general organization of topics within the present disclosure and are not intended to limit the disclosure of the technology or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. All references cited in the “Detailed Description” section of this specification are hereby incorporated by reference in their entirety.

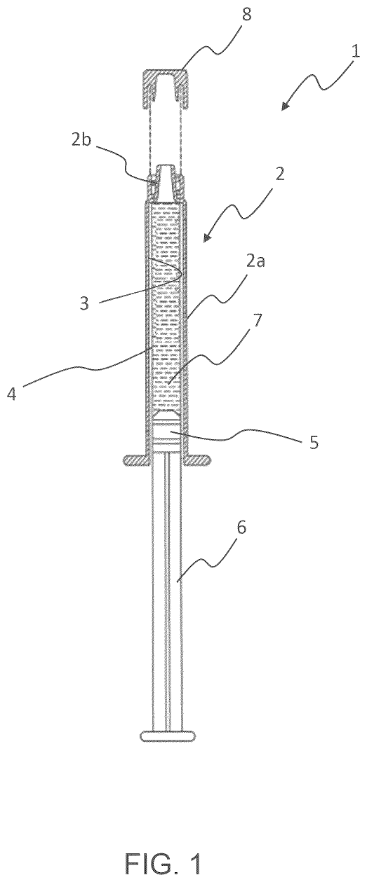

A medical injection device according to a preferred embodiment of the invention, in particular a syringe, is generally indicated by the reference numeral 1 in FIG. 1.

The term “syringe”, as used herein, is defined broadly in order to include cartridges, injection “pens” and other types of barrels or reservoirs adapted to be assembled with one or more other components to provide a functional syringe.

The term “syringe” also includes related articles such as self-injectors, which provide a mechanism for dispensing the content.

The syringe 1 comprises a syringe cylinder 2, made of glass, having a substantially cylindrical body 2a provided with a substantially conical end portion 2b.

The cylinder 2 has an inner surface 3 coated with a coating layer 4.

The cylinder 2 is also configured to receive a plunger 5 with sliding engagement.

In a way conventional per se, the plunger 5 is associated to one end of a drive stem 6.

In the preferred embodiment illustrated in FIG. 1, the syringe 1 further comprises an injectable liquid 7, for example a liquid pharmaceutical composition, within the cylinder 2 and in contact with the inner surface thereof 3.

The syringe 1 is also provided with a closing cap 8 of the end portion 2b of the cylinder 2 so as to allow the transport of the injectable liquid 7 in safe conditions.

In a preferred embodiment, the coating layer 4 comprises about 100% by weight of polydimethylsiloxane having a kinematic viscosity at room temperature equal to about 12500 cSt (125 cm²/s), for example the polydimethylsiloxane (PDMS) marketed under the name Liveo™ 360 Medical Fluid (DuPont).

The coating layer 4 of the syringe 1 illustrated in FIG. 1 comprises one or more of the features illustrated in the description above and which is referred to herein by reference.

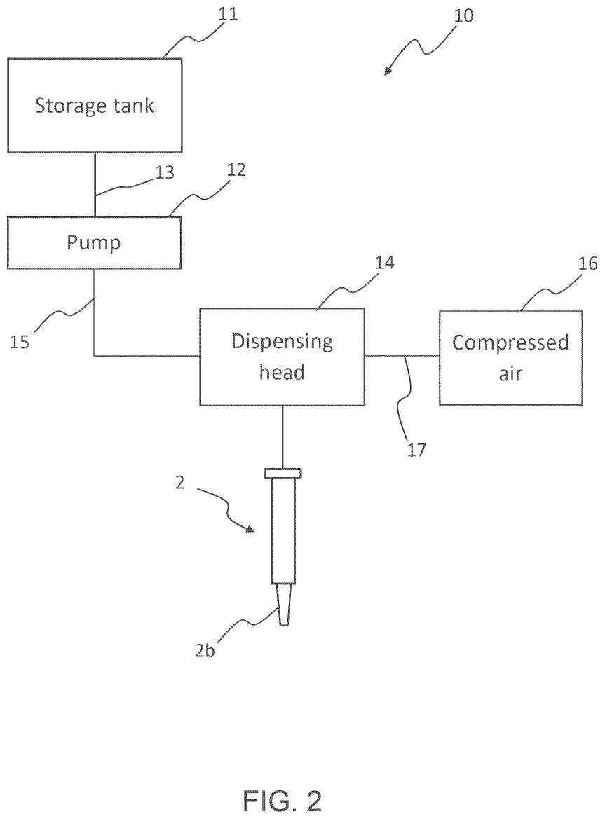

In a preferred embodiment, the syringe 1 may be manufactured by means of an apparatus 10 schematically illustrated in FIG. 2.

The apparatus 10 comprises a storage tank 11, preferably of stainless steel, for storing a coating composition provided with at least one heating element configured to heat the stored coating composition.

For example, the heating element of the tank 11 may be an electrical resistor or a pipe in which a suitable heating fluid circulates, placed inside the tank 11 itself or also an outer jacket of the tank 11 in which a suitable heating fluid circulates.

The tank 11 is in fluid communication with a circulation pump 12 of the coating composition by means of a pipe 13, preferably made of stainless steel, suitably insulated in a manner known per se.

In a preferred embodiment, the pump 12 comprises a respective heating element, not better shown in FIG. 2, configured to heat a delivery head of the pump 12, also not illustrated.

Merely by way of example, the heating element of the delivery head of the pump 12 may comprise one or more electrical resistors in heat exchange relationship with the delivery head 12 of the pump, for example incorporated in a respective casing, for example cylindrical, associated to the delivery head.

The pump 12 is in fluid communication with a dispensing head 14 configured to dispense the coating composition via a pipe 15, preferably made of stainless steel, suitably insulated in a manner known per se.

The dispensing head 14 is provided with at least one dispensing nozzle, not better shown in FIG. 2, configured to spray the coating composition onto the inner surface 3 of the cylinder 2 of the syringe 1.

The dispensing head 14 is provided with a respective heating element, also not better shown in FIG. 2, configured to heat the coating composition dispensed by the nozzle.

Merely by way of example, this heating element may be an electrical resistor in heat exchange relationship with the dispensing nozzle, for example incorporated in a casing, for example cylindrical, associated to the dispensing nozzle.

In this preferred embodiment of the apparatus 10, the storage tank 11, the pump 12 and the dispensing head 14 are therefore in fluid communication with each other via the pipes 13, 15.

In a particularly preferred embodiment, the pipes 13, 15 are in heat exchange relationship with a respective heating element, for example an electrical resistor or an outer jacket of the pipes in which a suitable heating fluid circulates.

In a manner known per se, the nozzle(s) of the dispensing head 14 are in fluid communication via a pipe 17 with a source 16 of a suitable dispensing gas, for example compressed air.

Preferably, the source 16 dispenses compressed air at a pressure of from 5 psi (0.34 bar) to 150 psi (10.34 bar), more preferably equal to about 30 psi (2.07 bar).

In a manner known per se, not better shown in FIG. 2, the apparatus 10 comprises a movable supporting frame of a plurality of cylinders 2 of respective syringes 1 of which one is schematically illustrated in FIG. 2.

The dispensing head 14 of the coating composition and the supporting frame of the cylinders 2 of the syringes 1 are movable relative to each other for inserting/extracting each nozzle of the dispensing head 14 in a respective cylinder 2 of said plurality of cylinders 2.

In a preferred embodiment, the relative movement between the dispensing head 14 and the supporting frame of the cylinder 2 is effected by moving the latter with respect to the dispensing head 14 which is fixed.

A preferred embodiment of a method of manufacturing a medical injection device, for example the syringe 1 illustrated above, comprises the following steps preferably carried out by means of the apparatus 10 illustrated in FIG. 2.

A first step comprises providing a coating composition comprising polydimethylsiloxane, for example comprising an amount equal to about 100% by weight of polydimethylsiloxane Liveo™ 360 Medical Fluid (DuPont) having a nominal kinematic viscosity at room temperature equal to about 12500 cSt (125 cm²/s).

Preferably, this step of providing the coating composition comprises storing the coating composition in the storage tank 11.

Preferably, the coating composition stored in the storage tank 11 is heated to a temperature of from 100° C. to 150° C., for example equal to about 120° C., by means of the heating element associated to the tank 11.

Preferably, the heated coating composition stored in the storage tank 11 is maintained at a pressure of from 5 psi (0.34 bar) to 150 psi (10.34 bar), preferably of from 10 psi (0.69 bar) to 30 psi (2.07 bar), even more preferably of from 10 psi (0.69 bar) to 15 psi (1.03 bar).

In a subsequent step, the heated coating composition is sent via the pump 12 to the dispensing head 14 equipped with at least one nozzle, preferably with a plurality of dispensing nozzles which provide for dispensing the heated coating composition onto the inner surface 3 of the cylinder 2 so as to form the coating layer 4 on said inner surface 3.

As explained above, the dispensing time of the heated coating composition onto the inner surface 3 of the cylinder 2 is of from 0.3 s to 1 s, preferably of from 0.4 s to 0.7 s.

The method comprises heating the dispensing head 14 and, more preferably, also the delivery head of the pump 12 and the pipes 13 and 15 so as to maintain the coating composition at the aforesaid temperature of from 100° C. to 150° C., for example equal to about 120° C., during the travel from the storage tank 11 to the nozzles of the dispensing head 14, which dispense the coating composition at the aforesaid temperature.

Preferably, the step of applying the heated coating composition at the aforesaid temperature onto the inner surface 3 of the cylinder 2 is carried out by dispensing the heated coating composition at a pressure of from 5 psi (0.34 bar) to 150 psi (10.34 bar), more preferably of from 6 psi (0.41 bar) to 10 psi (0.69 bar).

Preferably, this dispensing of the heated coating composition comprises feeding to the dispensing head 14 the dispensing air (gas) coming from the source 16 and having a pressure of from 5 psi (0.34 bar) to 150 psi (10.34 bar), preferably of from 6 psi (0.41 bar) to 10 psi (0.69 bar).

Preferably, the storage tank 11 of the coating composition is maintained at a pressure higher than the pressure of the nozzle(s) of the dispensing head 14 so as to optimize the dispensing of the heated coating composition.

Preferably, the step of applying the heated coating composition onto the inner surface 3 of the cylinder 2 comprises imparting a relative motion between the dispensing head 14 and the cylinder 2 while dispensing the heated coating composition.

Preferably, the step of applying the heated coating composition onto the inner surface 3 of the cylinder 2 comprises dispensing the heated coating composition onto the inner surface 3 of the cylinder 2 during a relative insertion movement of the dispensing head 14 into the cylinder 2.

Preferably, the step of applying the heated coating composition onto the inner surface 3 of the cylinder 2 comprises dispensing the heated coating composition at a flow rate of from 0.1 μL/s to 5 μL/s, for example at a flow rate of about 0.5 μL/s.

Preferably, the step of applying the heated coating composition onto the inner surface 3 of the cylinder 2 comprises applying onto said inner surface 3 an amount per unit area of heated coating composition of from 0.2 to 0.4 μg/mm².

Preferably, the step of applying the heated coating composition onto the inner surface 3 of the cylinder 2 is carried out such that the coating layer 4 formed on the inner surface 3 has an average thickness, measured by optical reflectometry, of from 100 to 250 nm, more preferably of from 100 to 200 nm.

In a preferred embodiment, the coating layer 4 formed on the inner surface of the cylinder has a thickness standard deviation, measured by optical reflectometry, equal to or less than 90 nm, preferably equal to or less than 70 nm, and, even more preferably, equal to or less than 50 nm.

In a preferred embodiment, for each batch of 10 cylinders 2, the batch average standard deviation SD, obtained as described above, of the thickness of the coating layer 4 has a value equal to or less than 70 nm, preferably equal to or less than 60 nm, and, even more preferably, equal to or less than 50 nm.

If desired, after the step of applying the heated coating composition onto the inner surface 3 of the cylinder 2, it is possible to carry out a further step of subjecting the coating layer 4 formed on the inner surface 3 of the cylinder 2 to a partial cross-linking treatment of the polydimethylsiloxane, for example carried out by irradiation by means of plasma torch at atmospheric pressure with an argon flow.

Preferably, the irradiation treatment is carried out for a time of from 0.2 s to 1 s, more preferably of from 0.2 to 0.6 s and, even more preferably of from 0.2 to 0.5 s, extremes included.

Preferably, the irradiation treatment is carried out at a time distance of at least 15 minutes, preferably of from 15 to 20 minutes, after the step of applying the heated coating composition onto the inner surface 3 of the cylinder 2.

If desired, before the step of applying the heated coating composition onto the inner surface 3 of the cylinder 2, it is possible to carry out a further step of subjecting the inner surface 3 of the cylinder 2 to a pre-treatment to improve adhesion of the coating layer 4 to the inner surface 2.

In a particularly preferred embodiment, this pre-treatment comprises forming on the inner surface 3 of the cylinder 2 a layer of an adhesion promoter, preferably a layer of an adhesion promoter comprising [(bicycloheptenyl)ethyl]trimethoxysilane.

If it is desired to manufacture a pre-filled syringe such as the one illustrated by way of example in FIG. 1, it is possible to carry out a further step of filling the cylinder 2 with the injectable liquid 7 after cooling the coating layer 4 formed on the inner surface 3 of the cylinder 2 to room temperature.

Finally, if it is desired to manufacture the pre-filled syringe 1 illustrated in FIG. 1, it is possible to carry out a further step of associating the cap 8 to the end portion 2b of the cylinder 2 so as to seal the content of the syringe 1.

The invention is now illustrated by means of some Examples thereof to be understood for exemplary and non-limiting purposes.

Again by way of illustration and not of limitation, in the following examples the medical injection devices (syringes) made according to the method according to the invention and having a nominal filling volume of 0.5 mL, 1 mL Long or 3 mL according to the ISO 11040-4 standard (2015) were manufactured by providing the following application conditions of the heated coating composition onto the inner surface 3 of the cylinders 2.

### Syringe of Nominal Filling Volume of 0.5 mL

Total stroke of each dispensing head 14 within each cylinder 2: 75 mm max

Speed of the dispensing head 14: 35 mm/s

Total cycle time (insertion/dispensing time+extraction time of the dispensing head 14): 2.1 s

Dispensing flow rate of the heated coating composition: 0.30 μL/s

Volume of dispensed coating composition: 0.30 μL

Dispensing time of the heated coating composition: 1 s.

### Syringe of Nominal Filling Volume of 1 mL

Total stroke of each dispensing head 14 within each cylinder 2: 80 mm max

Speed of the dispensing head 14: 52 mm/s

Total cycle time (insertion/dispensing time+extraction time of the dispensing head 14): 1.5 s

Dispensing flow rate of the heated coating composition: 0.63 μL/s

Volume of dispensed coating composition: 0.63 μL

Dispensing time of the heated coating composition: 1 s.

### Examples 1-2

### Manufacture of a Cylinder of a Medical Injection Device and Evaluation of the Thickness and Homogeneity of the Coating Layer Formed on the Inner Surface of the Cylinder-Syringes of Nominal Filling Volume of 1 mL or 3 mL

By means of the method and of the apparatus as described above, a coating composition heated to about 120° C. and consisting of PDMS Liveo™ 360 Medical Fluid (DuPont) having a nominal kinematic viscosity at room temperature of about 12500 cSt (125 cm²/s) was applied to the inner surface of the cylinder of a syringe of nominal filling volume of 1 mL (Example 1) or 3 mL (Example 2).

The storage tank was maintained at 120° C., the delivery head of the pump at about 50° C. and the nozzles of the dispensing head at about 120° C.

The deposited amount of silicone oil was approximately 0.2 μg/mm².

A coating layer was thus formed on the inner surface of the cylinder characterized by a very low thickness, constant over the entire axial extension of the body of the cylinder of the syringe as measured by means of an optical reflectometry method.

In particular, the thickness of the coating layer remained constant and on average less than 200 nm, preferably on average less than 150 nm, with an average value of from 120 to 160 nm for the entire axial length of the cylinder.

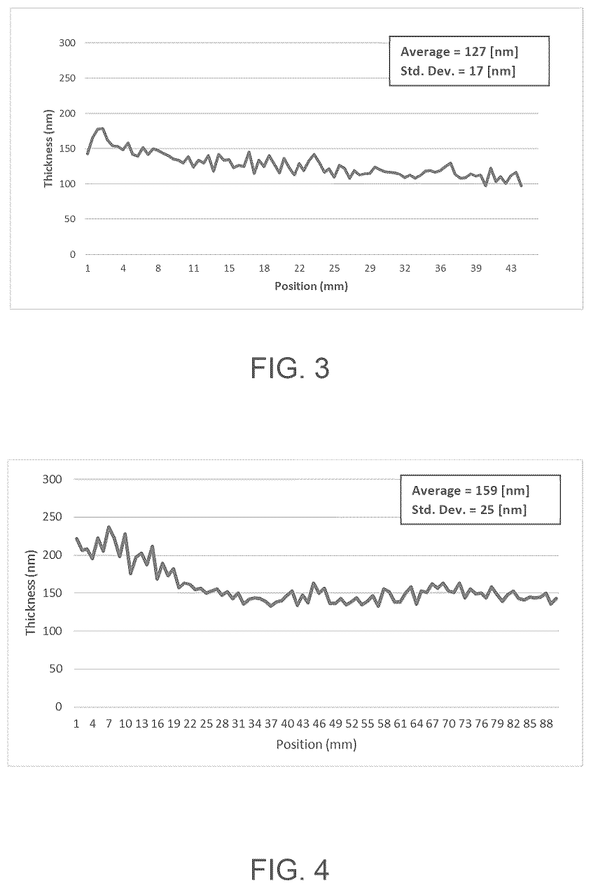

FIGS. 3 and 4 report the graphs resulting from the measurements carried out and illustrating the profile of the thickness of the coating layer applied to the inner surface of the cylinder of the syringe of nominal filling volume of 1 mL and 3 mL, respectively.

As can be seen from the aforesaid figures, the coating layer of the inner surface of the cylinder has a marked surface regularity as shown by the low value of the thickness standard deviation which is less than 30 nm in the case of the syringe of nominal volume of 3 mL (FIG. 4), and less than 20 nm in the case of the syringe of nominal volume of 1 mL (FIG. 3).

When subjected to a visual, possibly automated, inspection test, both syringes did not induce any evaluation errors.

### Examples A-G

### Manufacture of Syringes According to the Invention and Comparative Syringes

By means of the method and of the apparatus as described above, a heated coating composition consisting of PDMS Liveo™ 360 Medical Fluid (DuPont) having a nominal kinematic viscosity at room temperature of about 12500 cSt (125 cm²/s) was applied onto the inner surface of the cylinder of a syringe of nominal filling volume of 1 mL (A-B-C-D) and 3 mL (E-F-G).

By means of a conventional method and of a conventional apparatus, a coating composition consisting of PDMS Liveo™ 360 Medical Fluid (DuPont) having a nominal kinematic viscosity at room temperature of about 1000 cSt (10 cm²/s) was applied onto the inner surface of the cylinder of syringes of the same type.

The temperatures of the storage tank, of the delivery head of the pump and of the nozzles of the dispensing head, as well as the amount of silicone oil deposited are reported in Table 1 below.

A coating layer was thus formed on the inner surface of the cylinder characterized by a very low thickness, constant over the entire axial extension of the body of the cylinder of the syringe as measured by means of an optical reflectometry method.

The coating layers obtained were in some cases subjected to partial cross-linking by irradiation by means of a plasma torch at atmospheric pressure carried out with variable irradiation times and under the following conditions:
Maximum power output: 100 W
Gas used: Argon with purity greater than 99%
Argon flow rate: 7 SLM

The manufacturing parameters of the cylinders of the syringes are reported in Table 1 below.

TABLE 1

| | | Silicone | | | | | Amount of |
|---|---|---|---|---|---|---|---|
| | Pre- | material | Tank | Pump | Nozzle | Plasma | deposited |
| | treat- | viscosity | T | T | T | irradiation | material |
| Ex. | ment | (cSt) | (° C.) | (° C.) | (° C.) | time (s) | (μg/mm²) |
| A | Yes | 12500 | 120 | 60 | 120 | 0.3 | 0.39 |
| B | Yes | 12500 | 120 | 60 | 120 | 1 | 0.36 |
| C* | Yes | 1000 | RT | RT | 66 | 0.3 | 0.30 |
| D* | No | 1000 | RT | RT | RT | — | 0.38 |
| E | No | 12500 | 120 | 60 | 120 | — | 0.33 |
| F | No | 12500 | 120 | 60 | 120 | 0.3 | 0.32 |
| G* | No | 1000 | RT | RT | 66 | — | 0.28 |

*= comparative example
RT = room temperature
Silicone material with nominal kinematic viscosity of 12500 cSt (125 cm²/s) according to the invention: PDMS Liveo ™ Medical Fluid (DuPont)
Comparative silicone material with nominal kinematic viscosity of 1000 cSt (10 cm²/s): PDMS Liveo ™ 360 Medical Fluid 1000 cSt.

The following parameters were determined:
the average thickness S of the coating layers applied and the respective standard deviations measured after deposition and after cooling of the layers (t0);
the batch average standard deviation SD of the thickness of the coating layers of a batch of 10 syringes.

The results obtained are reported in Table 2 below.

TABLE 2

| | Average | Thickness | Batch average standard |
|---|---|---|---|
| | thickness | standard | deviation SD of the |
| Example | S (nm) | deviation (nm) | thickness (nm) |
| A | 109 | 37 | 34 |
| B | 138 | 48 | 59 |
| C* | 269 | 124 | 49 |
| D* | 353 | 76 | 71 |
| E | 178 | 70 | 29 |
| F | 132 | 60 | 42 |
| G* | 164 | 38 | 32 |

*= comparative example
The pre-treatment of the inner surface of the cylinders of the syringes, when present, was carried out by means of the steps of:
g1) nebulizing onto the inner surface of the cylinder a 2.2 wt % solution of [(bicycloheptenyl)ethyl]trimethoxysilane in isopropyl alcohol, by means of an ultrasonic static nozzle, with an amount of solution of from 5 to 80 μL depending on the cylinder size; and
g2) heating the cylinder thus treated in oven at a temperature of 140° C. for 20 minutes.

As can be seen from the data in Table 2 above, in the case of the syringes according to the invention the average thickness S of the coating layer has always been maintained at values below 180 nm with a thickness standard deviation equal to or less than 70 nm confirming a very high regularity of deposition.

The data of batch average standard deviation SD of the thickness of the coating layers calculated for a batch of 10 syringes, less than 60 nm, also confirm the high reproducibility of the method of manufacturing syringes according to the invention.

The syringes thus manufactured were subjected to some tests to evaluate the static and dynamic friction force, the release of particles and the morphological characteristics of the coating obtained. The results of these tests are reported below.

### Examples H-O

### Manufacture of Syringes According to the Invention and Comparative Syringes

By means of the method and of the apparatus as described above, a coating composition heated to about 120° C. and consisting of PDMS Liveo™ 360 Medical Fluid (DuPont) having a nominal kinematic viscosity at room temperature of about 12500 cSt (125 cm²/s) was applied onto the inner surface of the cylinder of a syringe of nominal filling volume of 0.5 mL.

By means of a conventional method and of a conventional apparatus, a comparative coating composition consisting of PDMS Liveo™ 360 Medical Fluid (DuPont) having a nominal kinematic viscosity at room temperature of about 1000 cSt (10 cm²/s) was applied onto the inner surface of the cylinder of syringes of the same type.

The temperatures of the storage tank, of the delivery head of the pump and of the nozzles of the dispensing head, as well as the amount of silicone oil deposited are reported in Table 3 below.

A coating layer was thus formed on the inner surface of the cylinder characterized by a very low thickness, constant over the entire axial extension of the body of the cylinder of the syringe measured by means of an optical reflectometry method.

The coating layers obtained were in some cases subjected to partial cross-linking by irradiation by means of a plasma torch at atmospheric pressure carried out with variable irradiation times and under the conditions referred to in the Examples A-G.

The manufacturing parameters of the cylinders of the syringes are reported in Table 3 below.

TABLE 3

| | | Silicone | | | | | Amount of |
|---|---|---|---|---|---|---|---|
| | Pre- | material | Tank | Pump | Nozzle | Plasma | deposited |
| | treat- | viscosity | T | T | T | irradiation | material |
| Ex. | ment | (cSt) | (° C.) | (° C.) | (° C.) | time (s) | (μg/mm²) |
| H | No | 12500 | 120 | 60 | 120 | — | 0.28 |
| I | No | 12500 | 120 | 60 | 120 | 0.3 | 0.29 |
| J | No | 12500 | 120 | 60 | 120 | 0.5 | 0.28 |
| K | Yes | 12500 | 120 | 60 | 120 | 0.3 | 0.26 |
| L | Yes | 12500 | 120 | 60 | 120 | 0.5 | 0.27 |
| M* | No | 1000 | RT | RT | 66 | — | 0.29 |
| N* | No | 1000 | RT | RT | 66 | 0.3 | 0.31 |
| O* | Yes | 1000 | RT | RT | 66 | 0.3 | 0.31 |

*= comparative example
RT = room temperature
Silicone material with nominal kinematic viscosity of 12500 cSt (125 cm²/s) according to the invention: PDMS Liveo ™ Medical Fluid (DuPont)
Comparative silicone material with nominal kinematic viscosity of 1000 cSt (10 cm²/s): PDMS Liveo ™ 360 Medical Fluid 1000 cSt

The following parameters were determined for the Examples H, I, K (invention) and M, N and O (comparative) after deposition and after cooling the layers (t0) and after a 3-month storage at room temperature (t3):
the average thickness S of the coating layers applied and the respective thickness standard deviations;
the batch average standard deviation SD of the thickness of the coating layers of a batch of 10 syringes.

The results obtained are reported in Table 4 below.

TABLE 4

| | Average | | Thickness | | Batch average | |
|---|---|---|---|---|---|---|
| | thickness | | standard | | standard deviation | |
| | S (nm) | | deviation (nm) | | SD of the thickness (nm) | |
| | Storage time (months) | | | | | |
| Example | 0 | 3 | 0 | 3 | 0 | 3 |
| H | 219 | 226 | 33 | 30 | 18 | 17 |
| I | 164 | 157 | 45 | 45 | 44 | 42 |
| K | 177 | 190 | 40 | 41 | 40 | 39 |
| M* | 273 | 264 | 75 | 77 | 53 | 61 |
| N* | 201 | 189 | 77 | 73 | 82 | 75 |
| O* | 201 | 217 | 83 | 75 | 90 | 74 |

*= comparative example

Furthermore, it has been experimentally observed that the maximum batch standard deviation of the thickness of the applied coating layers for the Examples H, I, K (invention) has always been maintained at a value less than 70 nm.

The pre-treatment of the inner surface of the cylinders of the syringes, when present, was carried out by means of the same methods described above with reference to the Examples A-G.

The syringes thus manufactured were subjected to some tests to evaluate the static and dynamic friction force, the particle release and the morphological characteristics of the coating obtained. The results of these tests are reported below.

### Evaluation of the Thickness of the Coating Layer

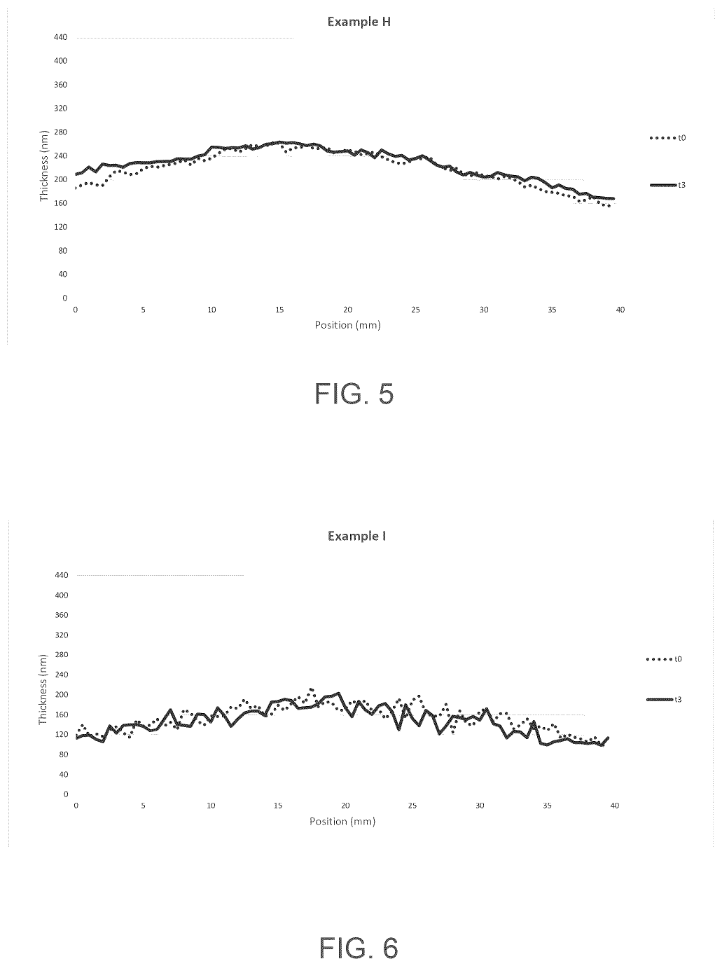

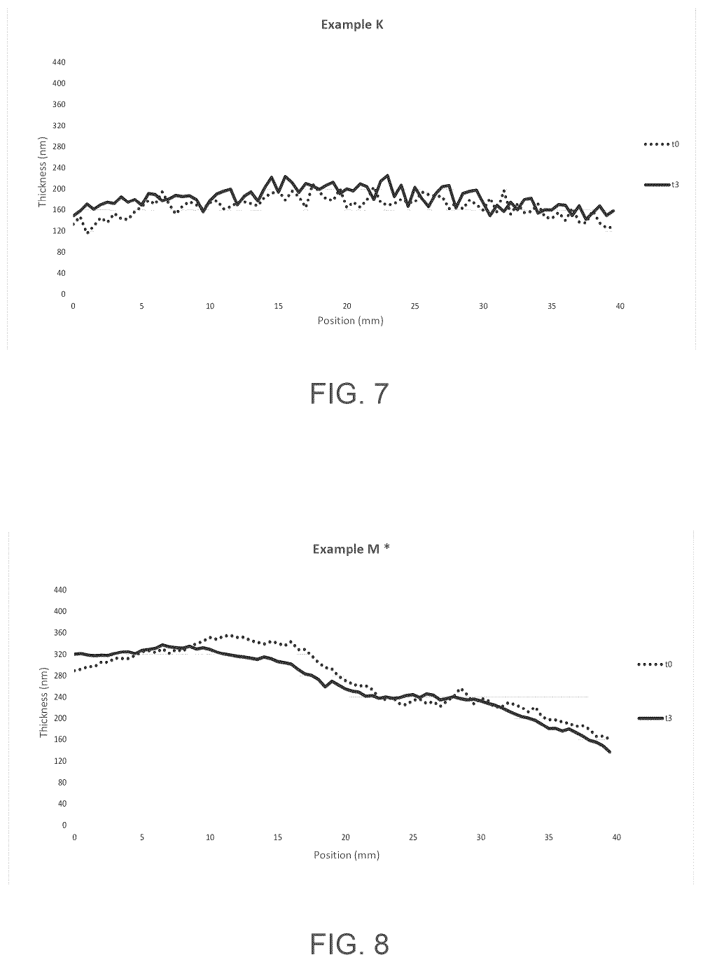

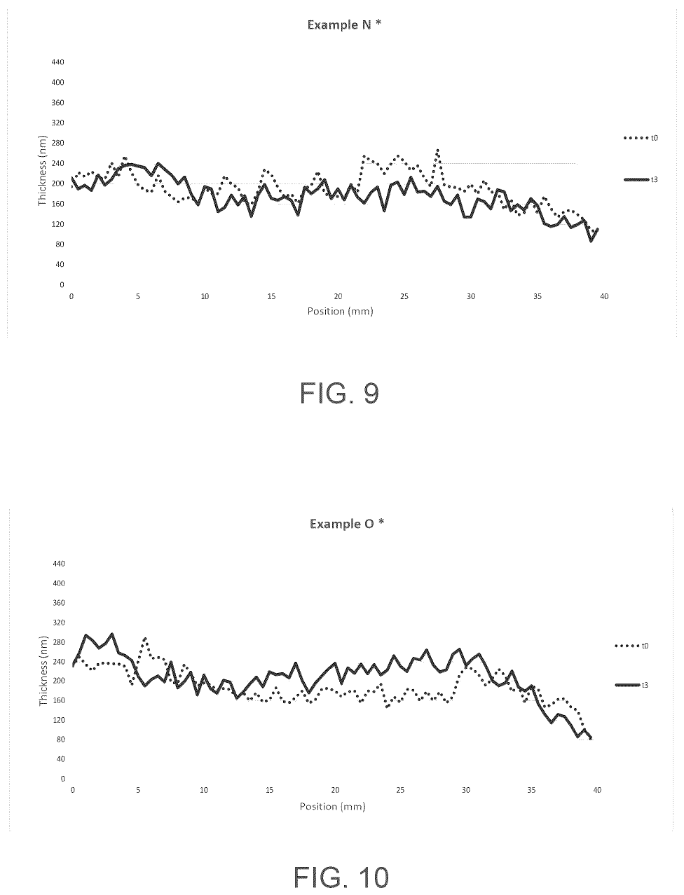

FIGS. 5-10 report the graphs resulting from the measurements carried out and illustrating the profile of the thickness of the coating layer applied to the inner surface of the cylinder of the syringe of nominal filling volume of 0.5 mL after deposition and cooling to room temperature (t0) and after a 3-month storage at room temperature (t3).

As can be seen from the data in the aforesaid Table 4 and from the aforementioned figures, in the case of the syringes according to the invention the coating layer of the inner surface of the cylinder has a low average thickness with a marked surface regularity.

The average thickness of the coating layer has in fact been maintained at values always lower than 230 nm with a thickness standard deviation of less than 50 nm confirming a very high regularity of the thickness of the coating layer.

In particular, as illustrated in Table 4, by comparing the syringes according to the invention with those according to the prior art without plasma treatment (example H vs. example M) it was experimentally found that there was a reduction of more than 50% in the thickness standard deviation confirming a marked improvement in the regularity of deposition of the coating layer despite the much higher kinematic viscosity of the silicone material used.

The values of batch average standard deviation SD of the thickness of the coating layers calculated for a batch of 10 syringes, less than 50 nm, also confirm the high reproducibility of the method of manufacturing a medical injection device according to the invention.

When subjected to an automated visual inspection test, the syringes according to the invention did not induce any evaluation error.

### Evaluation of the Average Values of the Static and Dynamic Sliding Friction Force on Empty Syringes Stored at Room Temperature

The syringes of the Examples A and B (invention) and C and D (comparative) were subjected to a series of comparative tests to evaluate the average values of the static and dynamic sliding friction force carried out on empty cylinders.

The syringes all had a nominal filling volume of 1.0 mL and the friction force was measured at room temperature at time zero and after a 6-month storage time at room temperature.

The measurement of the friction force was carried out with the following method using a ZwickiLine Z2.5 (Zwick Roell) dynamometer.
Place the syringe in the appropriate support of the dynamometer.
Reset the load cell force (not under pressure)
Set a constant speed deformation of 240 mm/min, a preload of 0.5 N and an end stop at a preset force of 30 N
Start the test (30 samples/example) and measure the resulting force

By analysing the curve resulting from the dynamometer, the static friction force was identified as the force corresponding to the first initial peak and the dynamic friction force as the mean of the values of the zone between the first initial peak and the end stop peak.

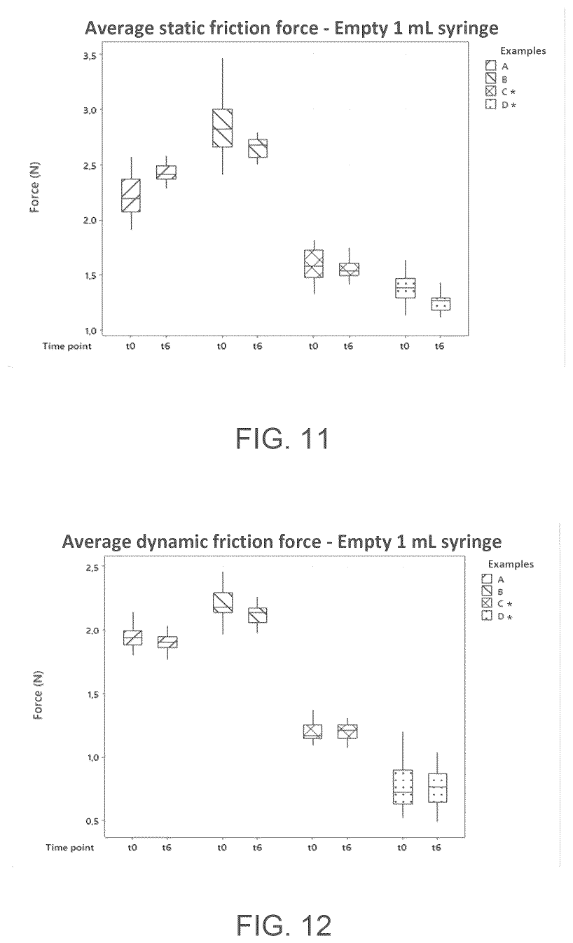

The average values of the static and dynamic friction force measured on batches of 30 syringes are reported in FIG. 11 and, respectively, in FIG. 12.

As can be seen from the aforesaid figures, the average values of the static and dynamic friction force for the syringes according to the invention (Examples A and B) with coating layers of the cylinder subjected to various irradiation times are entirely acceptable and within the limit values previously indicated required by the pharmaceutical and cosmetic industry (6N for the static sliding friction force and 3N for the dynamic sliding friction force).

It is also noted that the maximum acceptable irradiation time of the coating layer of the cylinder is of the order of 1 s.

### Evaluation of the Average Values of Static and Dynamic Sliding Friction Force on Syringes Filled and Stored at Room Temperature—Empty Syringes of Nominal Filling Volume of 1 mL Long

The syringes of the Examples A and B (invention) and C and D (comparative) were subjected to a further series of comparative tests to evaluate the average values of the static and dynamic sliding friction force carried out on cylinders having a nominal filling volume of 1.0 mL filled with an aqueous test solution (injectable liquid) comprising water and glycerol (volumetric fraction of glycerol of from 0.02% vol to 0.04% vol) to achieve a dynamic viscosity of 1 mPa·s (1 cP) that simulates the behaviour of a medicament.

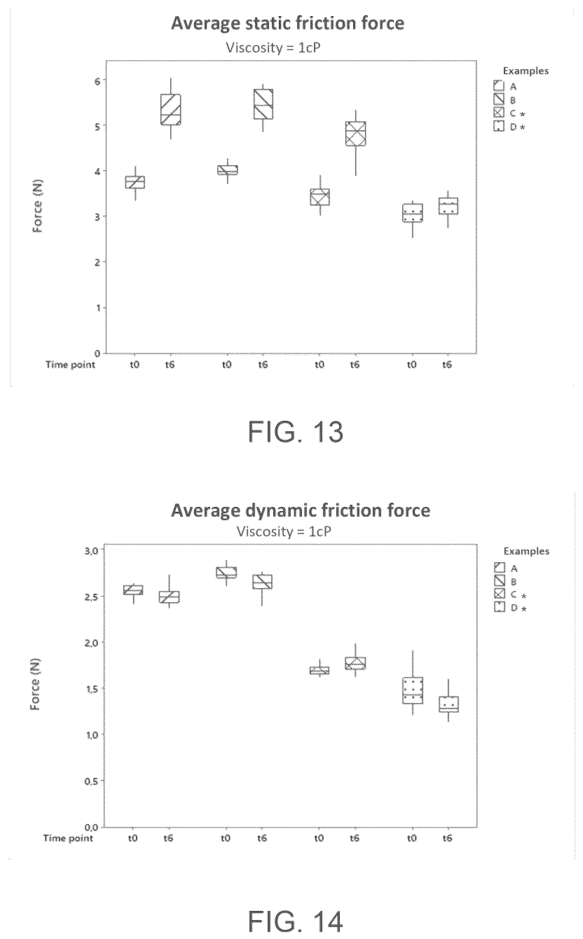

The tests were carried out under the same conditions as those on the empty syringes and gave average values of the static and dynamic friction force measured on batches of 30 syringes reported in FIG. 13 and, respectively, in FIG. 14.

Also in this case, the average values of the static and dynamic friction force for the syringes according to the invention (Examples A and B) with coating layers of the cylinder subjected to various irradiation times were still acceptable (6N for the static sliding friction force and 3N for the dynamic sliding friction force).

Also in this case, the maximum acceptable irradiation time of the cylinder coating layer was found to be of the order of 1 s.

Evaluation of the Average Values of the Static and Dynamic Sliding Friction Force on Filled Syringes after a 7-Day Storage at Different Temperatures—Syringes of Nominal Filling Volume of 1 mL Long

The syringes of the Examples E and F (invention) and D (comparative) were subjected to a series of comparative tests to evaluate the average values of the static and dynamic sliding friction force carried out on cylinders of nominal filling volume of 1.0 mL filled with 0.55 mL of a test aqueous solution (injectable liquid) having the following composition:
Tromethamine 0.34 mg.
Tromethamine hydrochloride 1.30 mg.
Acetic acid 0.047 mg.
Sodium acetate 0.132 mg.
Sucrose 47.85 mg.
Water for injectable preparations, balance up to 0.55 mL

The friction force was measured as indicated above at room temperature (RT) and at temperatures of −20° C. and −40° C., after a 7-day storage time.

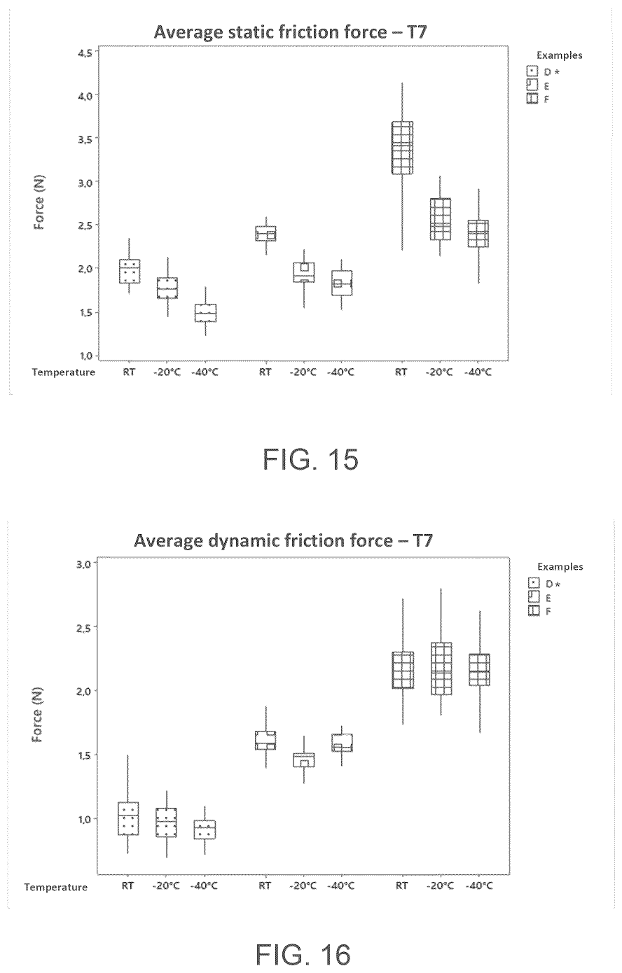

The average values of the static and dynamic friction force measured on batches of 30 syringes are reported in FIG. 15 and, respectively, in FIG. 16.

As can be seen from the aforesaid figures, the average values of the static and dynamic friction force for the syringes according to the invention (Examples E and F) with coating layers of the cylinder not subjected to irradiation (Example E) or subjected to irradiation for a time of 0.3 s (Example F) are comparable with those of a comparative syringe (Example D) provided with a coating layer of known type (silicone material with nominal kinematic viscosity of about 1000 cSt).

Furthermore, the average values of the static and dynamic friction force for the syringes according to the invention (Examples E and F) fully fall within the limit values previously indicated required by the pharmaceutical and cosmetic industry (6N for the static sliding friction force and 3N for the dynamic sliding friction force).

Evaluation of the Average Values of the Static and Dynamic Sliding Friction Force on Filled Syringes after a 2- and 7-Day Storage at a Temperature of −40° C.—Syringes with Nominal Filling Volume of 1 mL Long

The syringes of the Examples E and F (invention) and D (comparative) were subjected to a further series of comparative tests to evaluate the average values of the static and dynamic sliding friction force carried out on cylinders with nominal filling volume of 1.0 mL filled with 0.55 mL of the test aqueous solution (injectable liquid) having the following composition:
Tromethamine 0.34 mg.
Tromethamine hydrochloride 1.30 mg.
Acetic acid 0.047 mg.
Sodium acetate 0.132 mg
Sucrose 47.85 mg.
Water for injectable preparations, balance up to 0.55 mL

The friction force was measured as indicated above after a 2- and 7-day storage time at −40° C.

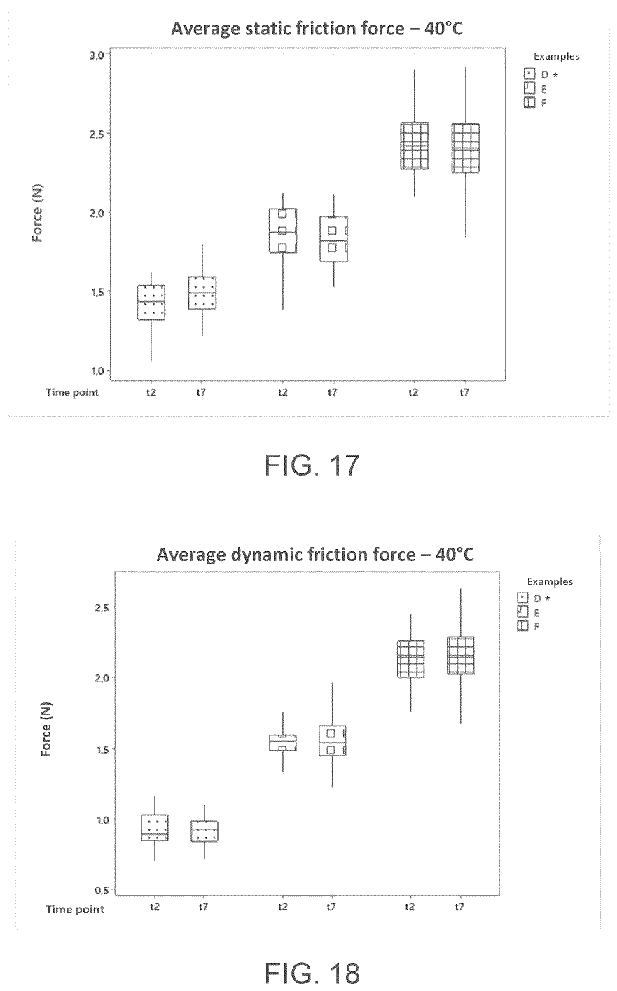

The average values of the static and dynamic friction force measured on batches of 30 syringes are reported in FIG. 17 and, respectively, in FIG. 18.

As can be seen from the aforesaid figures, the average values of the static and dynamic friction force for the syringes according to the invention (Examples E and F) with coating layers of the cylinder not subjected to irradiation (Example E) or subjected to irradiation for a time of 0.3 s (Example F) are comparable with those of a comparative syringe (Example D) provided with a coating layer of known type (silicone material with nominal kinematic viscosity of about 1000 cSt).

Furthermore, the average values of the static and dynamic friction force for the syringes according to the invention (Examples E and F) were substantially stable and such as to fully fall within the limit values previously indicated required by the pharmaceutical and cosmetic industry (6N for the static sliding friction force and 3N for the dynamic sliding friction force).

### Evaluation of the Average Values of the Static and Dynamic Sliding Friction Force on Empty Syringes Stored at Room Temperature—Empty Syringes of Nominal Filling Volume of 0.5 mL Long

The syringes of the Examples H, I, J, K and L (invention) and M, N and O (comparative) were subjected to a series of comparative tests to evaluate the average values of the force of static and dynamic sliding friction carried out on empty cylinders.

The syringes all had a nominal filling volume of 0.5 mL and the friction force was measured at room temperature at time zero and after a 1- and 3-month storage time at room temperature.

The measurement of the friction force was carried out with the following method using a ZwickiLine Z2.5 (Zwick Roell) dynamometer.
Place the syringe in the appropriate support of the dynamometer.
Reset the load cell force (not under pressure)
Set a deformation at the constant speed of 100 mm/min, without setting a preload and a stop end at a preset force of 30 N
Start the test (30 samples/example) and measure the resulting force.

By analysing the curve resulting from the dynamometer, the static friction force was identified as the force corresponding to the first initial peak and the dynamic friction force as the mean of the values of the zone between the first initial peak and the end stop peak.

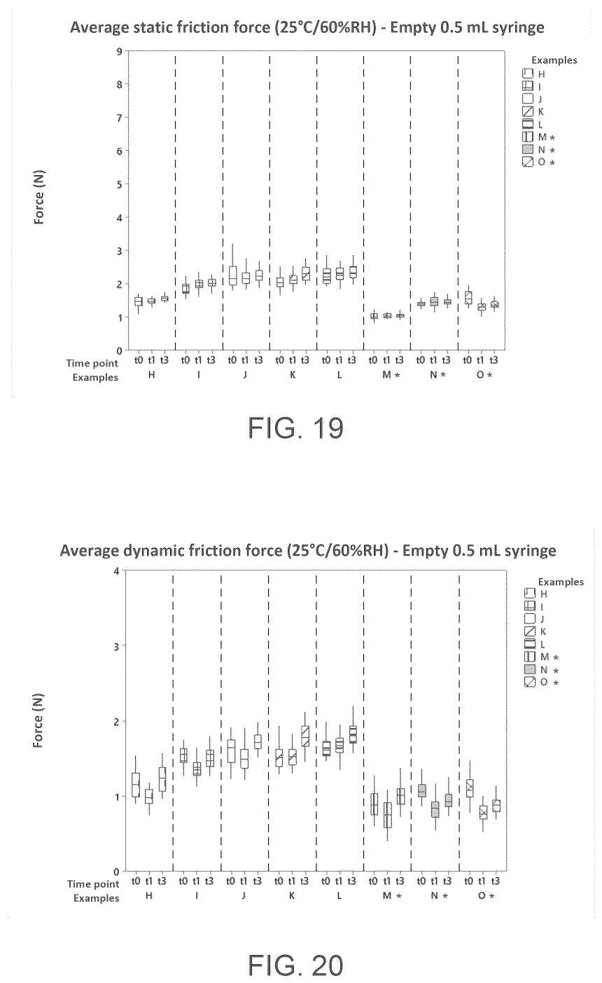

The average values of the static and dynamic friction force measured on batches of 30 syringes are reported in FIG. 19 and, respectively, in FIG. 20.

As can be seen from the aforesaid figures, the average values of the static and dynamic friction force for the syringes according to the invention (Examples H, I, J, K and L) with coating layers of the cylinder not subjected to irradiation (Example H) or subjected to various irradiation times (Examples I, J, K and L) are completely acceptable and within the limit values previously indicated required by the pharmaceutical and cosmetic industry (6N for the static sliding friction force and 3N for the dynamic sliding friction force).

### Evaluation of the Average Values of the Static and Dynamic Sliding Friction Force on Filled Syringes Stored at a Temperature of −40° C., +5° C., +25° C. and +40° C.—Syringes of Nominal Filling Volume of 0.5 mL

The syringes of the Examples H, I, J, K and L (invention) and M, N and O (comparative) were subjected to a further series of comparative tests to evaluate the average values of the static and dynamic sliding friction force carried out on cylinders of nominal filling volume of 0.5 mL filled with 500 μL of test aqueous solution (injectable liquid) having the following composition:
10 mM sodium phosphate.
40 mM sodium chloride
0.03% (v/v) Polysorbate 20
   5% (w/v) Sucrose
Water for injectable preparations (filtered MilliQ aqueous solution with 0.22 μm filter diameter) balance up to 0.5 mL and pH 6.2.

The friction force was measured as indicated above after deposition and cooling of the coating layer (t0) and after a storage time of 1 month (t1) and 3 months (t3) at the following temperatures; −40° C., +5° C., +25° C. and +40° C.

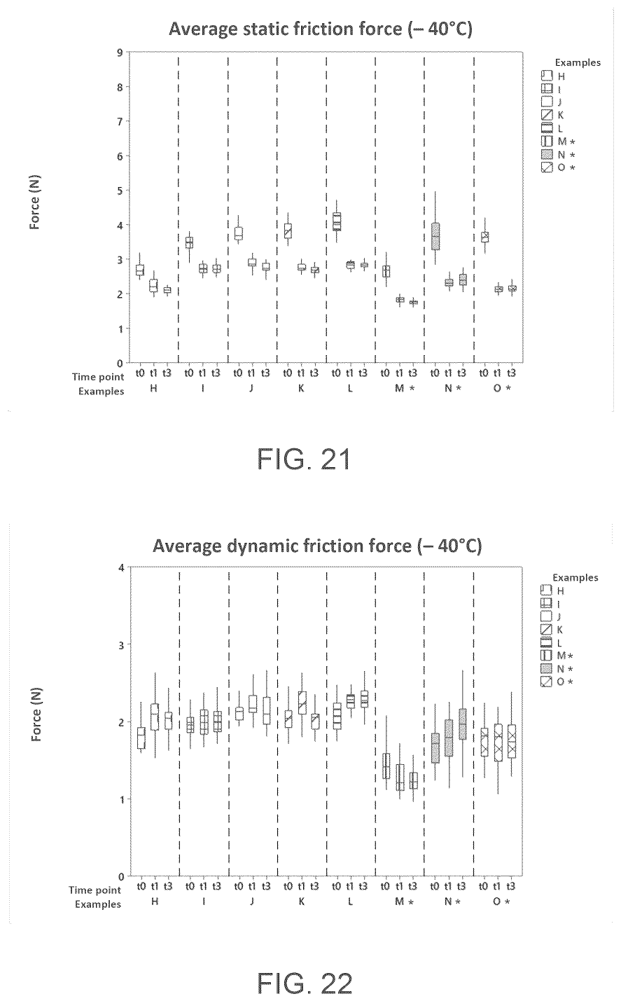

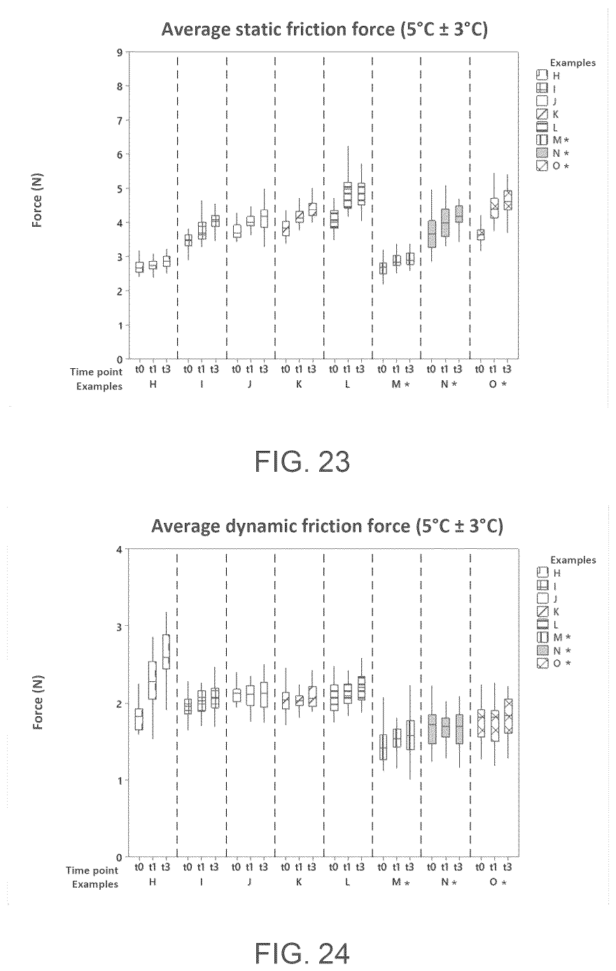

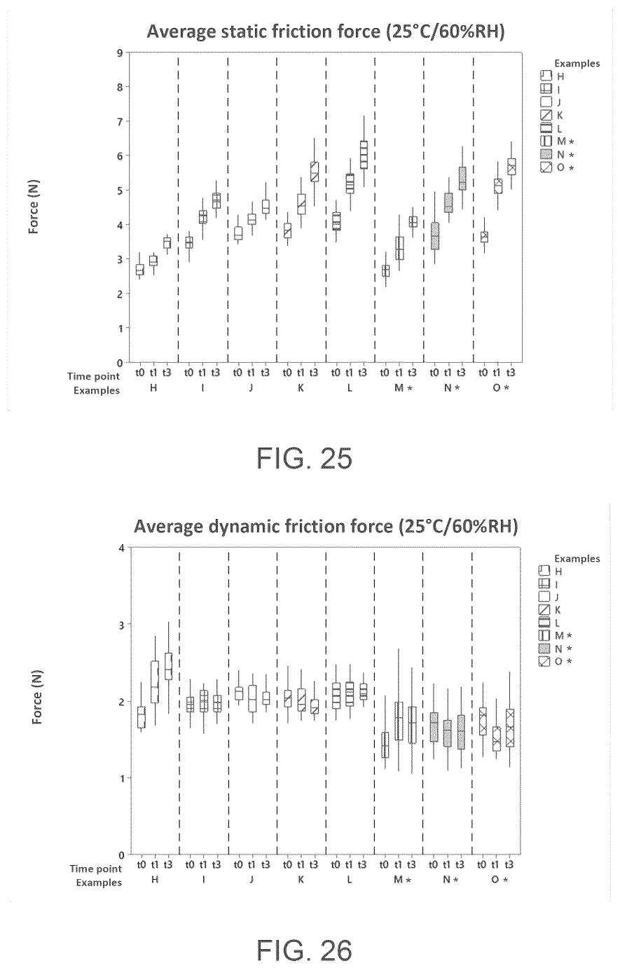

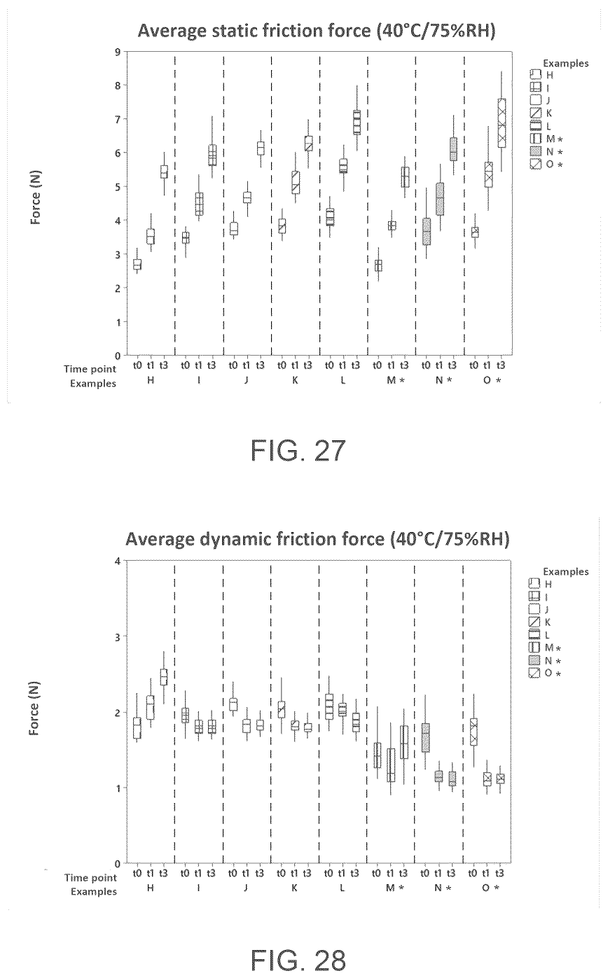

The average values of the static and dynamic friction force measured on batches of 30 syringes are reported in FIGS. 21-28.

As can be seen from the aforesaid figures, the average values of the static and dynamic friction force for the syringes according to the invention (Examples H, I, J, K and L) with coating layers of the cylinder not subjected to irradiation (Example H) or subjected to irradiation for a time of 0.3 s or 0.5 s (Examples K, I, J and L) are comparable with those of comparative syringes (Examples M, N and O) provided with coating layer of known type (silicone material with nominal kinematic viscosity of about 1000 cSt).

Furthermore, the average values of the static and dynamic friction force for the syringes according to the invention (Examples H, I, J, K and L) were substantially stable and such as to fully fall within the limit values previously indicated required by the pharmaceutical and cosmetic industry (6N for the static sliding friction force and 3N for the dynamic sliding friction force).

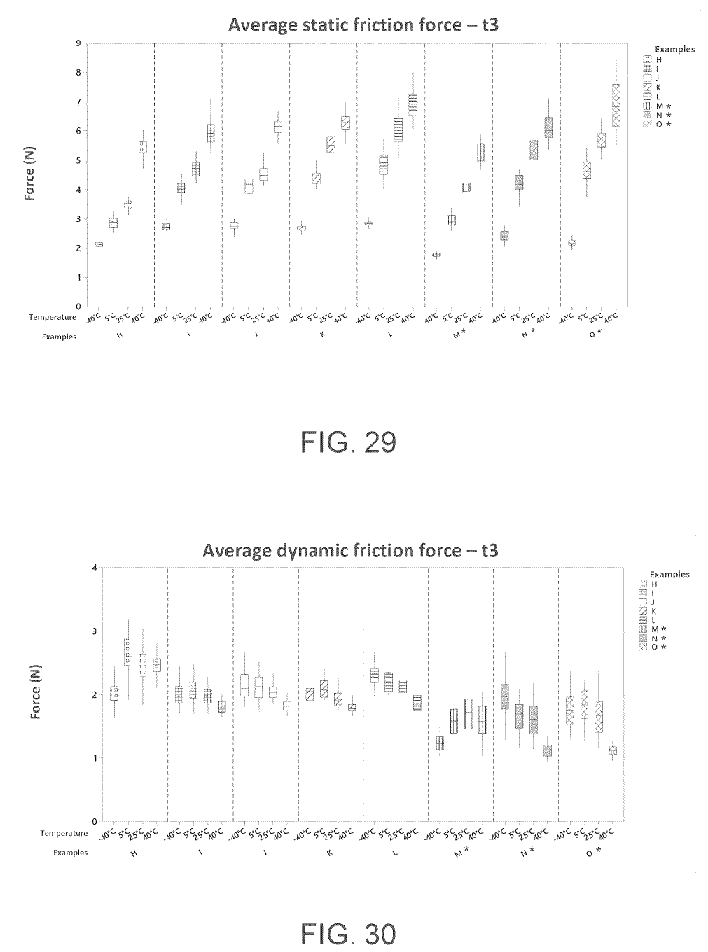

The average values of the static and dynamic friction force of the plunger of the syringes according to the invention and according to the prior art after a three-month storage at the aforesaid temperatures of −40° C., +5° C., +25° C. and +40° C. are further reported by way of comparison in FIGS. 29 and 30.

As can be seen from the aforesaid figures, after a three-month storage at various temperatures, the average values of the static and dynamic friction force for the syringes according to the invention (Examples H, I, J, K and L) are comparable with those of the comparative syringes (Examples M, N and O) provided with a coating layer of known type and fully falling within the limit values previously indicated required by the pharmaceutical and cosmetic industry (6N for the static sliding friction force and 3N for the dynamic sliding friction force).

### Evaluation of the Particle Release on Filled Syringes at Room Temperature

The syringes of the Example E (invention) and of the Examples C and G (comparative) were subjected to a series of comparative tests to evaluate the release of particles in a test aqueous solution (injectable liquid). The syringes all had a nominal filling volume of 3.0 mL and were filled with 3.3 mL of a test aqueous solution (injectable liquid) having the following composition:
10 mM sodium phosphate (adjusted to pH 7.0 using phosphoric acid).
0.9% (w/v) sodium chloride.
0.02% (w/v) polysorbate 80.
Water for injectable preparations balance up to 3.3 mL

### Preparation of the Samples for the Test of Particle Analysis

Fill the syringe cylinder with the test solution and close the cylinders with a plunger.
Storage (if envisaged by the test).
End-over-end rotation of the syringes (i.e. rotation about an axis perpendicular to the longitudinal axis of the cylinders) by means of a multi-rack agitator for 3 h with a rotation speed of 30 rpm
Dispensing of the aqueous test solution from the cylinders of the syringes: automated via dynamometer

The test liquid is collected in special containers.

Aliquots of sample solutions (pools) were obtained having a volume of at least 6 mL of liquid on which to carry out the particle analysis (e.g. 2 syringes filled with 3.30 mL result in 1 pool=1 sample for particle analysis).

The measurement of the concentration of the particles released in the test solution was performed by means of the method described below.

### Analysis of the Particles Released in the Test Solution—Examples A-G

Light Obscuration (LO) method

The test solution pools as obtained above were analysed by a Light Obscuration apparatus (KL 04A, RION) for the determination of sub-visible particle size and count.

This instrument performs particle counting in the analysed solution according to USP standard (787-788-789) as described in US Pharmacopeia 44-NF39 (2021).

In particular, the solution is aspirated from the instrument by means of a special needle and passes through a laser light source. The particles in solution induce the blockage of the beam of laser light and therefore a signal that is sent to the sensor; the size of the particles is given by the amount of obscured light.

The dimensional range that can be determined by the instrument ranges from 1.3-100 μm.

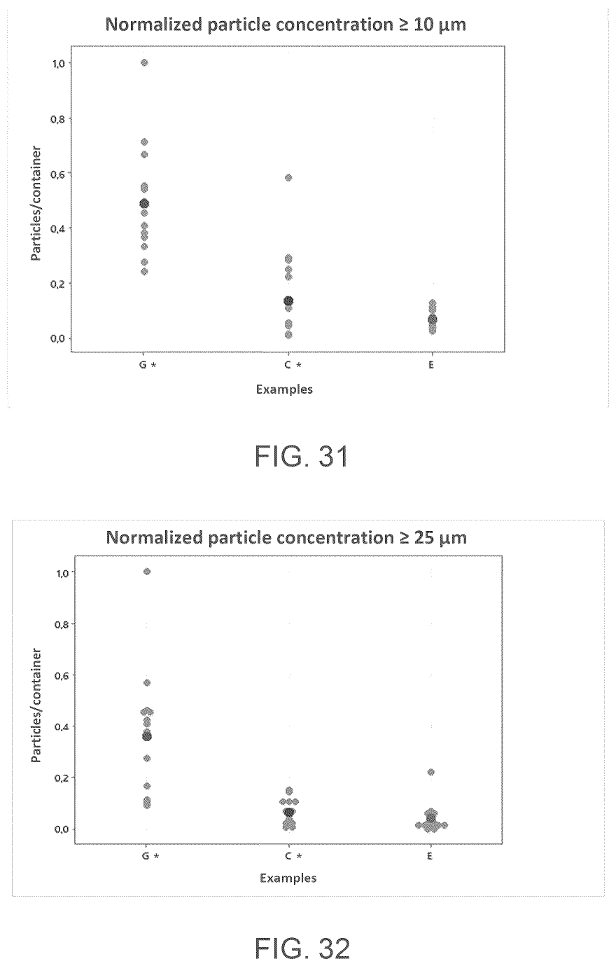

The normalised values of the concentration, measured at room temperature and immediately after rotation of the syringes, of particles with a size equal to or greater than 10 μm and equal to or greater than 25 μm obtained on 15 measurement pools starting from 30 syringes are reported in FIG. 31 and, respectively, in FIG. 32.

As can be seen from the above figures, the syringes according to the invention (Example E) with a coating layer of the cylinder not subjected to irradiation showed an improved particle release behaviour with respect to comparative syringes (Examples C and G) with coating layers of the cylinder respectively subjected to irradiation for 0.3 s (Comparative example C) or not subjected to irradiation (Comparative example G).

Evaluation of the Release of Particles at Different Temperatures on Filled Syringes without and with Storage

The syringes of the Example A (invention) and C (comparative) were subjected to a series of comparative tests to evaluate the release of particles in a test aqueous solution (injectable liquid).

The syringes all had a nominal filling volume of 0.5 mL and were filled with 0.25 mL of a test aqueous solution (injectable liquid) having the following composition:
10 mM sodium phosphate.
40 mM sodium chloride.
0.03% (w/v) polysorbate 20.
Sucrose 5% (w/v).
Water for injectable preparations (filtered MilliQ aqueous solution with 0.22 μm filter diameter) balance up to 0.5 mL and pH 6.2.

### Preparation of the Samples for the Particle Analysis Test

Fill the syringe cylinder with the test solution and close the cylinders with a plunger.
Storage
End-over-end rotation of the syringes (i.e. rotation about an axis perpendicular to the longitudinal axis of the cylinders) by means of a multi-rack agitator for 3 h with a rotation speed of 30 rpm
Dispensing of the test aqueous solution from the cylinders of the syringes: manual under laminar flow hood

The measurement of the concentration of particles released in the test solution was carried out by means of the method described below.

### Analysis of the Particles Released in the Test Solution

### LO (Light Obscuration)

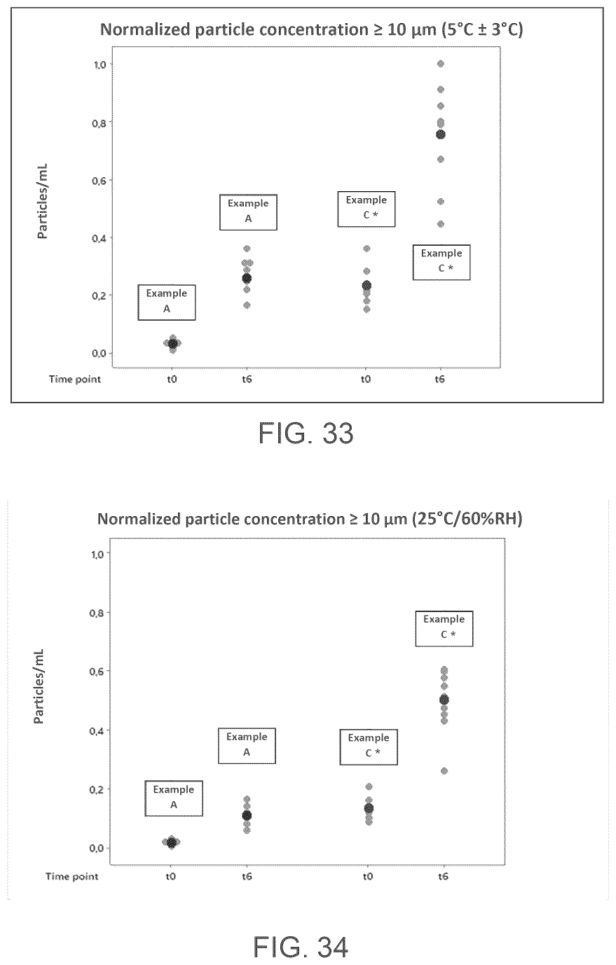

The normalised values of the concentration measured at time zero after preparation and after a storage for 6 months at the temperatures of 5° C.±3° C., 25° C./60% RH and 40° C./75% RH of particles with a size equal to or greater than 10 μm obtained on 12 pools (prepared by grouping two by two the solutions dispensed manually from 24 syringes in total) are reported in FIGS. 33, 34 and, respectively, 35.

As can be seen from the aforesaid figures, the syringes according to the invention (Example A) with a coating layer of the cylinder subjected to irradiation for 0.3 s have shown a clearly improved particle release behaviour with respect to the comparative syringes (comparative Example C) also with a coating layer of the cylinder subjected to irradiation for 0.3 s.

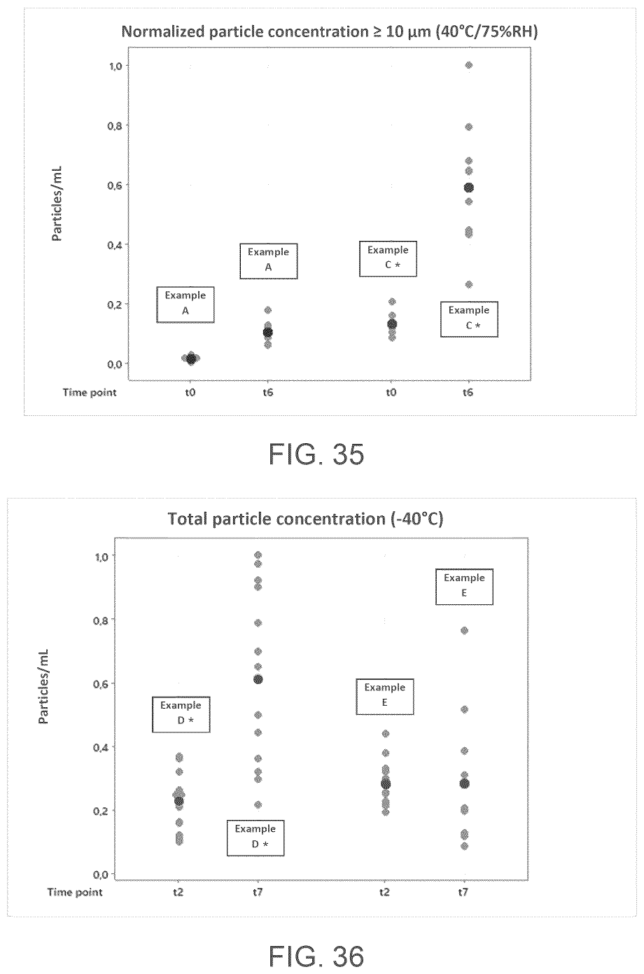

The particle release values illustrated in FIGS. 33-35 also show that the syringes according to the invention show an improved release stability over time after storage at various temperatures with respect to the comparative syringes.

Evaluation of the Release of Particles on Filled Syringes with Low-Temperature Storage

The syringes of the Examples E (invention) and D (comparative) were subjected to a series of comparative tests to evaluate the release of particles in a test aqueous solution (injectable liquid).

The syringes all had a nominal filling volume of 1.0 mL and were filled with 0.55 mL of an aqueous solution (injectable test fluid) having the following composition:
Tromethamine 0.34 mg.
Tromethamine hydrochloride 1.30 mg.
Acetic acid 0.047 mg.
Sodium acetate 0.132 mg
Sucrose 47.85 mg.
Water for injectable preparations, balance up to 0.55 mL

### Preparation of the Samples for the Particle Analysis Test

Fill the syringe cylinder with the test solution and close the cylinders with a plunger.
Storage.
Dispensing the aqueous test solution from the cylinders of the syringes: automated via dynamometer

The measurement of the particles released in the test solution was carried out by means of the following method.

### Analysis of the Particles Released in the Test Solution

### MFI (Micro Flow Imaging)

1 mL of each pool as obtained above was analysed by a flow imaging analysis apparatus (MFI™ Micro-Flow Imaging, MFI 5200, ProteinSimple) to evaluate the morphology of the particles in solution, thanks to the optical system of the instrument that is able to discriminate the different types of particles (particles of silicone material and not) based on certain parameters such as circularity and light intensity.

The specific parameters used to discriminate the particles of silicone material were as follows:
Aspect Ratio≥0.83 (i.e. ratio of the length of the minor axis to the length of the major axis of an ellipse having the same second-moments of the particle);
Intensity STD≥185 (i.e. standard deviation of intensity of all pixels representing the particle);
ECD 10-25 μm and 25-100 μm (i.e. diameter of a circle occupying the same area as the particle).

The dimensional range that can be determined by the instrument is 2-70 μm with a good resolution of the images of the particles with a size greater than 10 μm.

The normalised values of the concentration of the particles with a size of 5-70 μm measured after a 2- and 7-day storage at −40° C. and obtained on 15 measurement pools (prepared by grouping two by two the solutions dispensed with a dynamometer of 30 syringes in total) are reported in FIG. 36.

As can be seen from the above figure, the syringes according to the invention (Example E) with a cylinder coating layer not subjected to irradiation showed a comparable (after a 2-day storage) or clearly improved (after a 7-day storage) particle release behaviour with respect to the comparative syringes (Example D) also with cylinder coating layer not subjected to irradiation.

The particle release values illustrated in FIG. 36 also show that the syringes according to the invention show an improved release stability over time after a low-temperature storage with respect to the comparative syringes.

Evaluation of the Release of Particles on Filled Syringes with Storage at Various Temperatures-Syringes of Nominal Filling Volume of 0.5 mL—Examples H-O

The syringes of the Examples H, I, J, K and L (invention) and M, N and O (comparative) were subjected to a series of comparative tests to evaluate the release of particles in an aqueous test solution (injectable liquid). The syringes all had a nominal filling volume of 0.5 mL and were filled with 500 μL of an aqueous test solution (injectable liquid) having the following composition:
10 mM sodium phosphate.
40 mM sodium chloride.
0.03% (v/v) Polysorbate 20.
5% (w/v) Sucrose.
Water for injectable preparations (filtered MilliQ aqueous solution with 0.22 μm filter diameter) balance up to 0.5 mL and pH 6.2.

### Preparation of the Samples for the Particle Analysis Test

Filling of the cylinder of the syringes with the test solution and closure of the cylinders with a plunger (plunger 4023/50 Grey Flurotec, Westar).
Storage at different temperatures.
5° C.±3° C.
25° C./60% RH
40° C./75% RH
−40° C.
For syringes stored at −40° C., thawing was carried out before dispensing the solution, for one hour at room temperature, without end-over-end rotation. This was done in order to simulate a real situation of use of the products generally stored at this temperature, i.e. biotech drugs very sensitive to temperature.
End-over-end rotation of the syringes (i.e. rotation about an axis perpendicular to the longitudinal axis of the cylinders) by multi-rack agitator for 3 h with rotation speed equal to 30 rpm.
Dispensing of the test aqueous solution from the cylinders of the syringes: manual under laminar flow hood, grouping the solutions of 12 syringes in total.

The measurement of the concentration of particles released in the test solution was performed by means of the method described below.

### Analysis of the Particles Released in the Test Solution

### LO (Light Obscuration)

5 mL of each of the 10 pools (prepared by pooling the manually dispensed solutions of 12 syringes in total) were analysed by a particle count analysis apparatus (Light Obscuration particle counter KL-04A, Rion Co., LTD.).

This apparatus allows to operate according to USP <787>, <788>, <789> as described in US Pharmacopeia 44-NF39 (2021), and Ph. Eur. 2.9.19 (10^{th} edition, 2021) for subvisible particle count analysis of parenteral solutions.

The size of the analysed particles is determined by the amount of laser light of the source obscured by the particle itself when it passes through the laser beam, thus generating a voltage variation, which is detected by the sensor.

The size range of the particles that can be analysed by the apparatus is of from 1.3 to 100 μm.

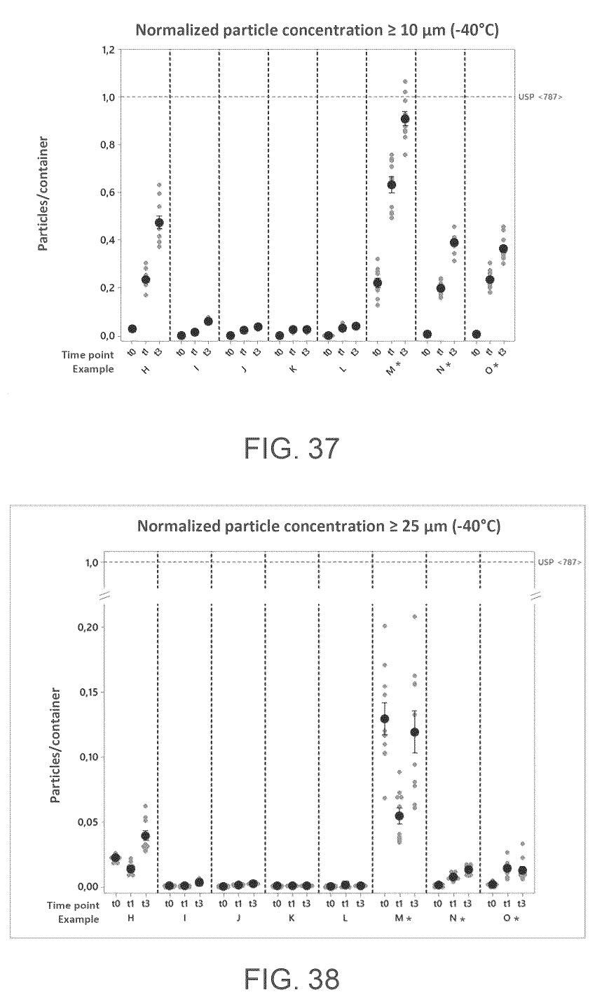

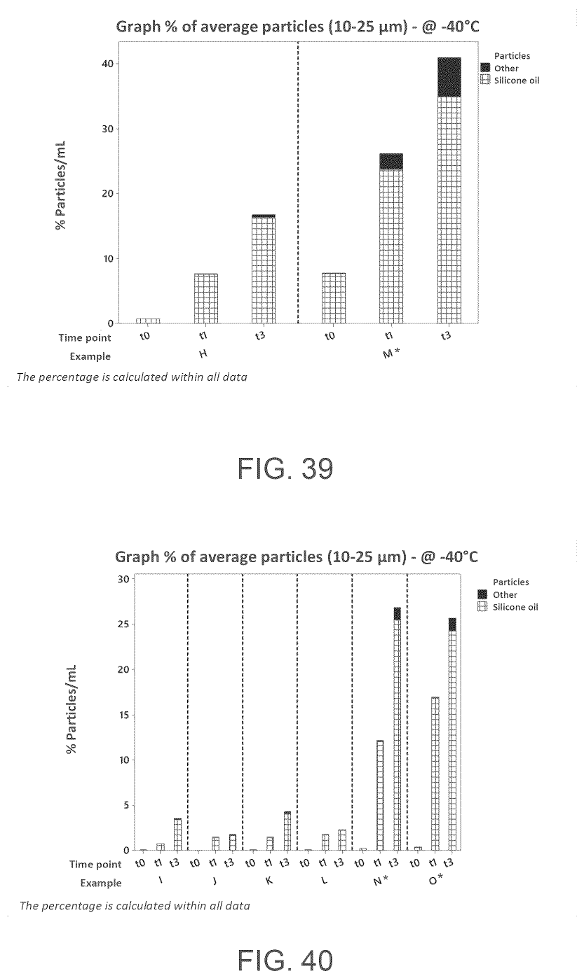

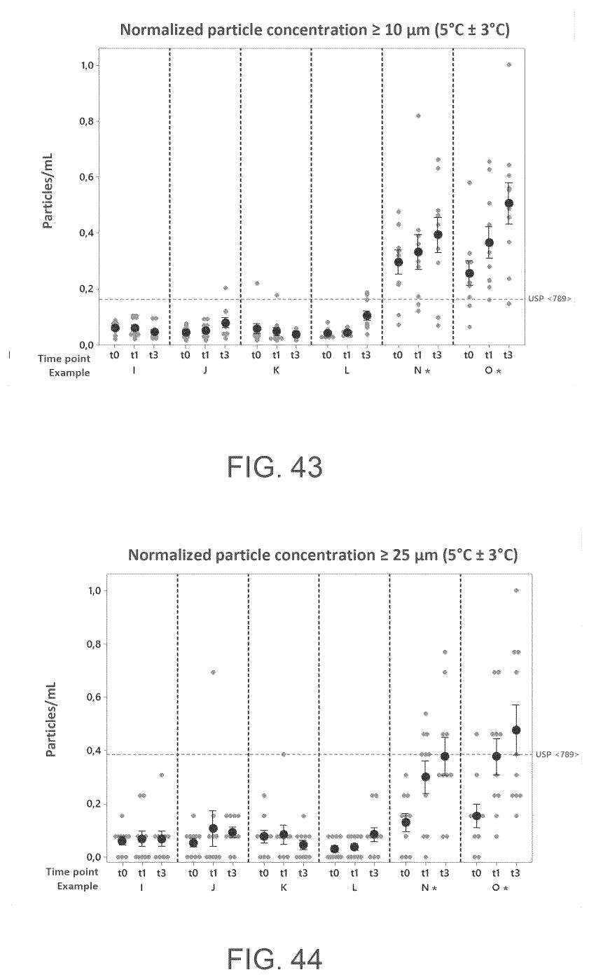

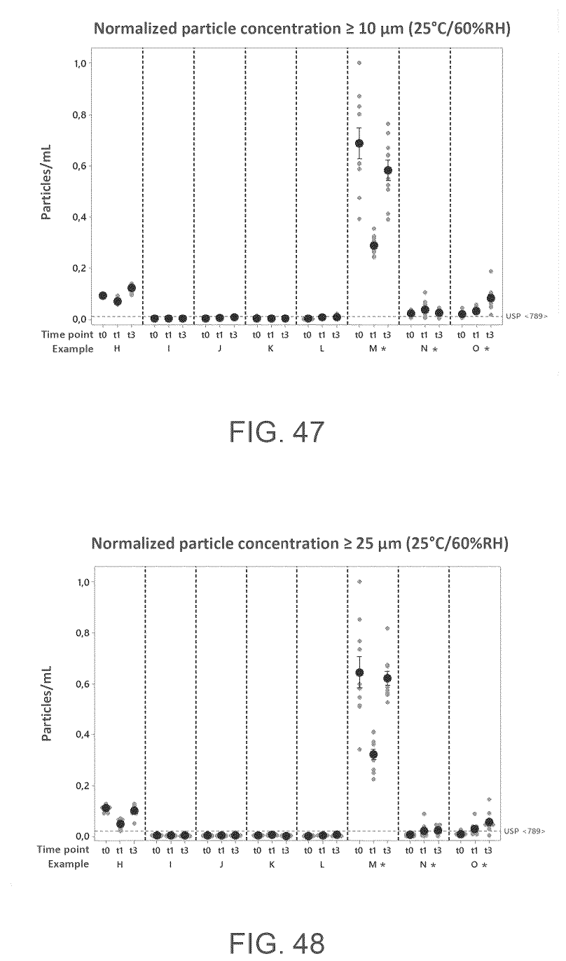

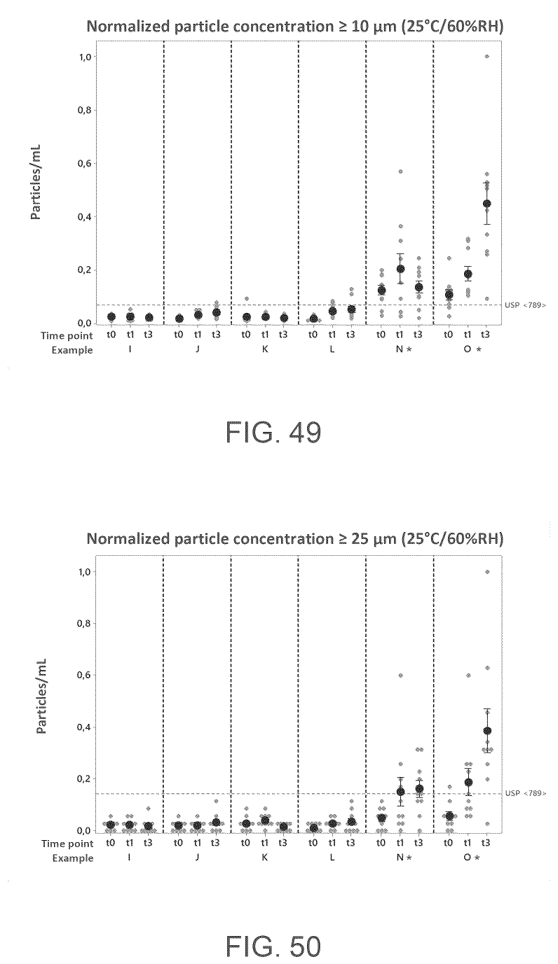

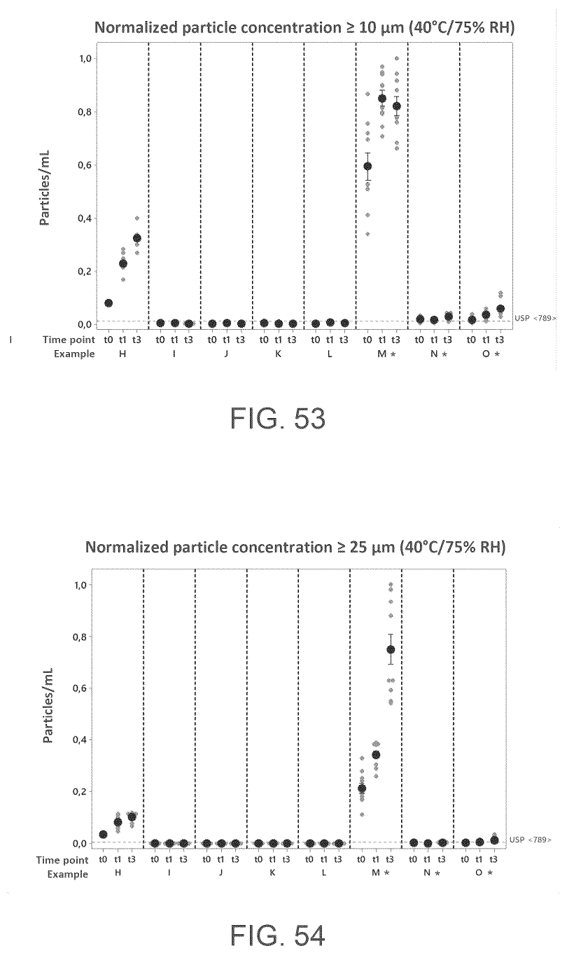

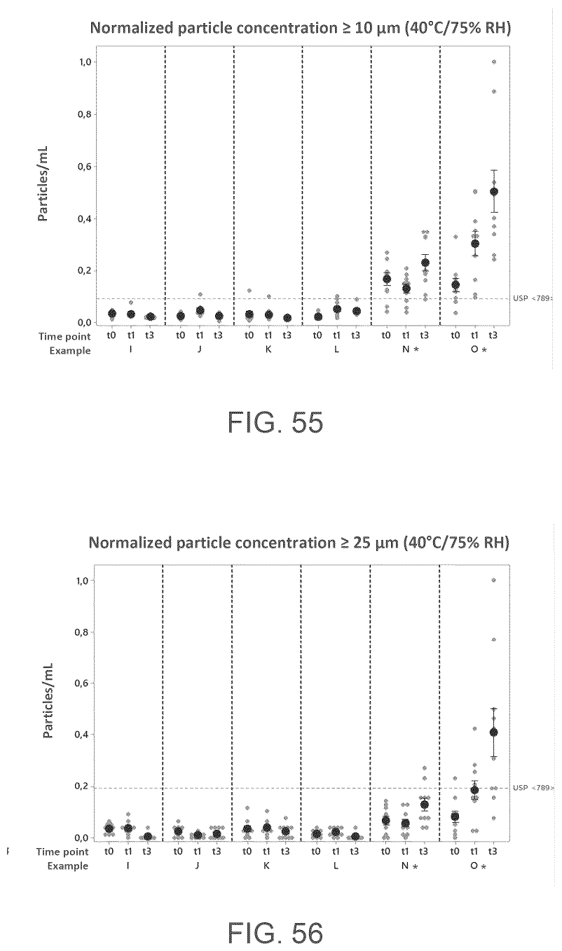

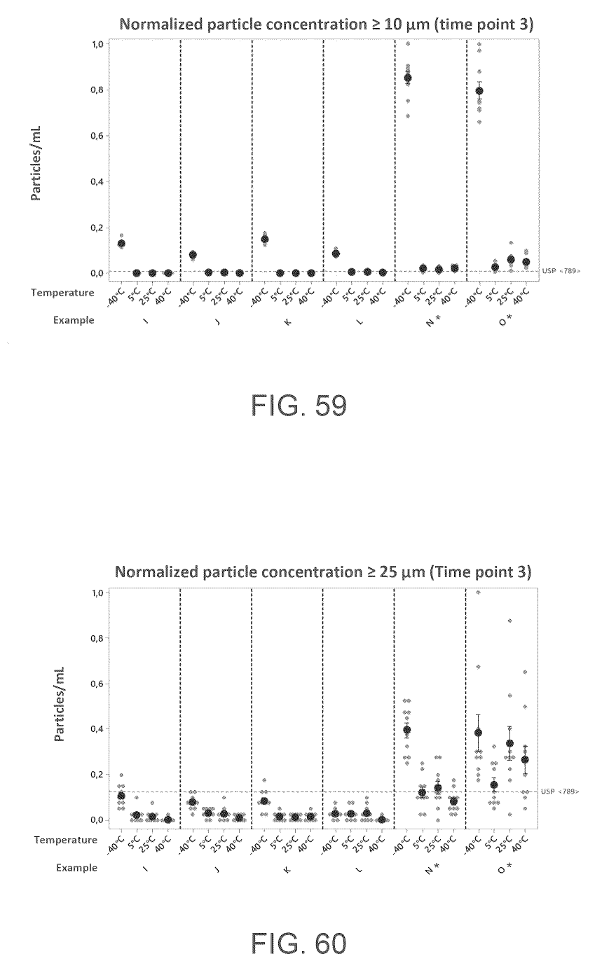

The normalised values of the concentration measured at time zero after preparation and after storage for 1 month and 3 months at the temperatures of −40° C., 5° C.±3° C., 25° C./60% RH, 40° C./75% RH, of particles with sizes equal to or greater than 10 μm and equal to or greater than 25 μm obtained on 10 pools are reported in FIGS. 37, 38, 41-44, 47-50, 53-56 and 59-60.

As can be seen from the aforesaid figures, at all the detection times (t0, t1 and t3) and at all the storage temperatures, the syringes according to the invention (Examples H, I, J, K and L) showed a clearly improved particle release behaviour with respect to the comparative syringes (Examples M, N and O), in particular employing a storage temperature of −40° C. and as better illustrated in FIGS. 37 and 38.

In particular, as illustrated in the above figures, by comparing the syringes according to the invention with those according to the prior art under the same process conditions, that is with or without plasma treatment and with or without pre-treatment to improve adhesion of the coating layer to the inner surface of the cylinders of the syringes, it has been experimentally found that:
there was a reduction by about 70% in the particle release in the case of coating layers not treated with plasma and with syringes not subjected to an adhesion pre-treatment (Example H vs. Example M);
there was a reduction by about 86% in the particle release in the case of coating layers treated with plasma for a time of 0.3 s and with syringes not subjected to adhesion pre-treatment (Example I vs. Example N);
there was a reduction by about 90% in the particle release in the case of coating layers treated with plasma for a time of 0.3 s and with syringes subjected to an adhesion pre-treatment (Example K vs. Example O).

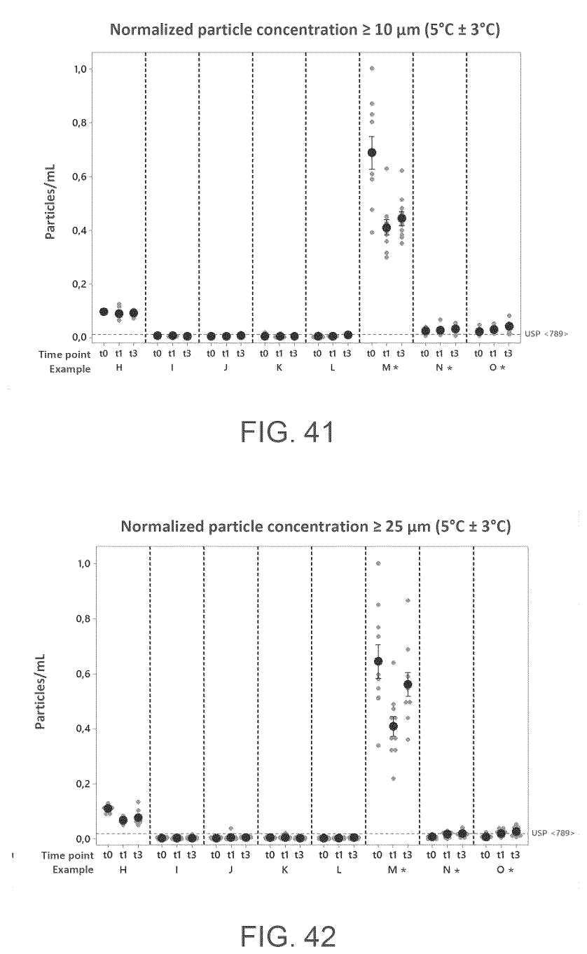

Furthermore, and as better illustrated in FIGS. 41-44, 47-50 and 53-56, by comparing the syringes according to the invention with a coating layer subjected to plasma treatment, with or without pre-treatment (Examples I, J, K and L), with those according to the prior art with the same treatment (Examples N and O), it was experimentally found that all the syringes according to the invention meet the stringent particle release requirements of the USP 789 standard for ophthalmic applications at all temperatures and storage times tested, a result which instead never occurs in the case of syringes of the prior as far as the particles of size equal to or greater than 10 μm are concerned (see FIGS. 41, 43, 47, 49, 53, 55 and 59).

Conversely, as to the particles of size equal to or greater than 25 μm, all the syringes according to the invention with a coating layer subjected to plasma treatment, with or without pre-treatment (Examples I, J, K and L) meet the particle release requirements of USP 789 standard at all temperatures and storage times tested, a result which occurs only in some cases for the syringes according to the prior art (Examples N and O). In particular, after a 3-month storage time, the syringes of comparative Example N meet the particle release requirements of standard USP 789 only for storage temperatures of 5° C. and 40° C., whereas the syringes of comparative Example O do not meet the particle release requirements of standard USP 789 at any of the storage temperatures (see FIGS. 42, 44, 48, 50, 54, 56 and 60).

### MFI (Micro Flow Imaging)

1 mL of each pool as obtained above for the 0.5 mL syringes was analysed by means of a flow imaging apparatus (MFI™ Micro-Flow Imaging, MFI 5200, ProteinSimple) to evaluate the morphology of the particles in solution as described above.

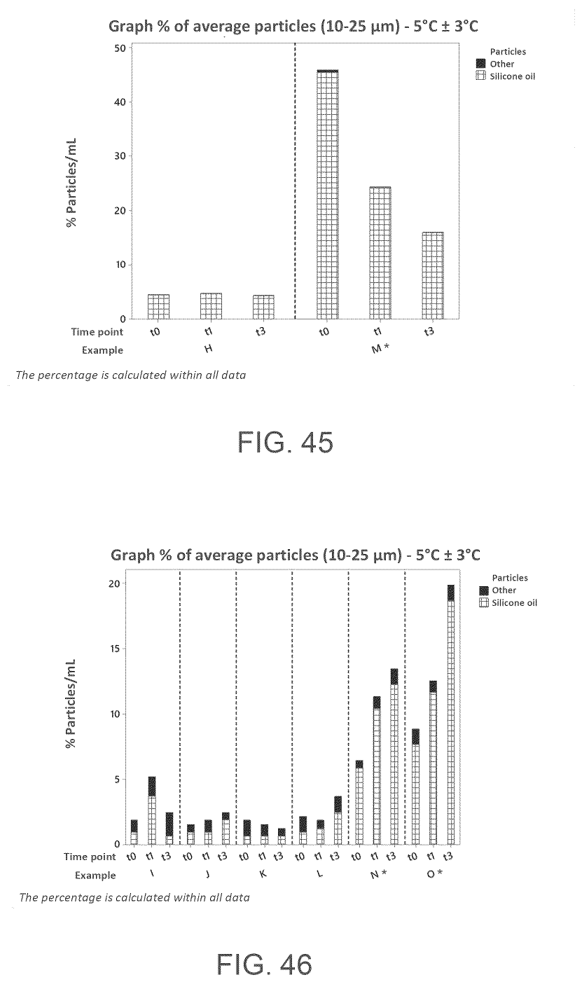

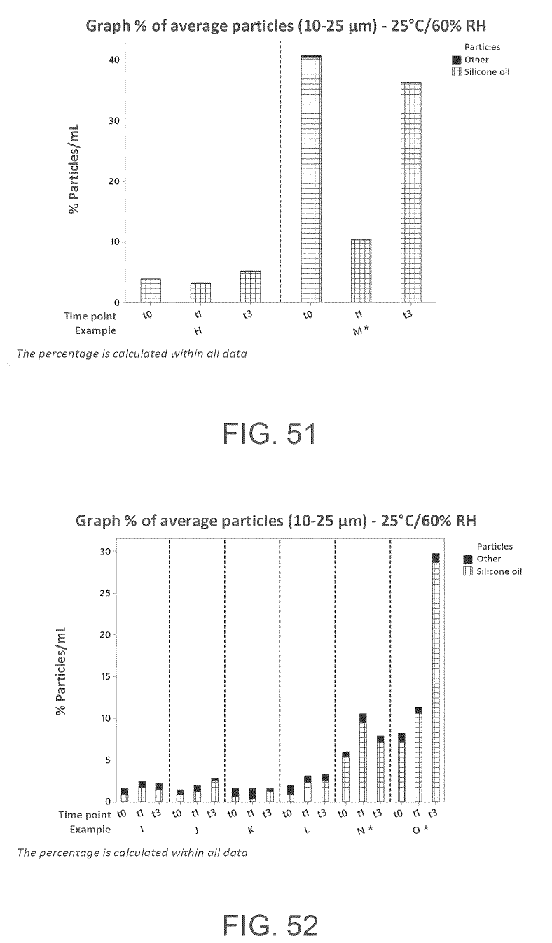

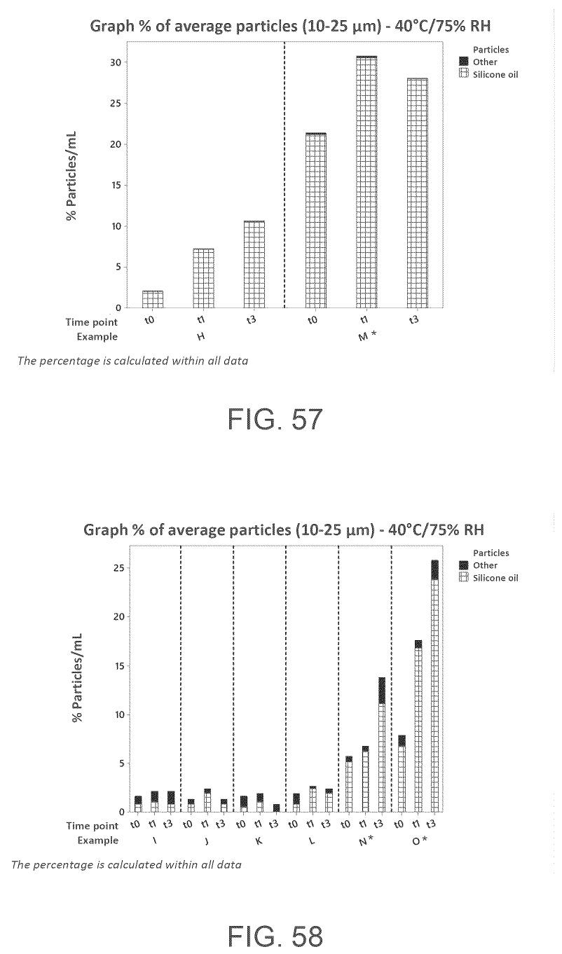

The percentage values (calculated within the examples) of the concentration of particles with size 10-25 μm measured at time zero after preparation and after storage for 1 month and 3 months at the temperatures of −40° C., +5° C.±3° C., +25° C./60% RH and +40° C./75% RH, obtained on 10 samples (obtained by taking 1 mL of solution from each pool prepared as above) are reported in FIGS. 45-46, 51-52 and 57-58.

As can be seen from the aforesaid figures, the syringes according to the invention (Examples H, I, J, K and L) allowed to drastically reduce the release of silicone particles with respect to the comparative syringes (Examples M, N and O) at all temperatures and at all test detection times (t0, t1 and t3).

### Evaluation of the Morphological Characteristics of Coating Layers Applied to the Inner Surface of the Cylinder of Empty Syringes

In order to evaluate the effects on the morphology of the coating layer that can occur at different times of irradiation of a coating layer obtained according to the invention and according to the prior art, some images were acquired by means of an optical microscope.

In general, the more the surface of the coating layer is homogeneous, or with a very fine granularity, the better it appears from the morphological point of view and, therefore, the less the surface will be prone to mislead an automated optical inspection system generating problems of false positives due to a surface irregularity of the coating layer.

In this regard and as explained above, the Applicant has observed that the degree of partial cross-linking related, for example, to the irradiation time in a plasma treatment, is critical insofar as it generates streaks and detachments that can be erroneously “read” by an automated optical inspection system as impurities present in the solution stored in the cylinder of the medical injection device.

The Applicant has observed that these streaks and detachments tend to first arise in the area of the cone-shaped portion (closest to the end where the needle is positioned) of the syringe cylinder and then propagate towards the cylindrical portion.

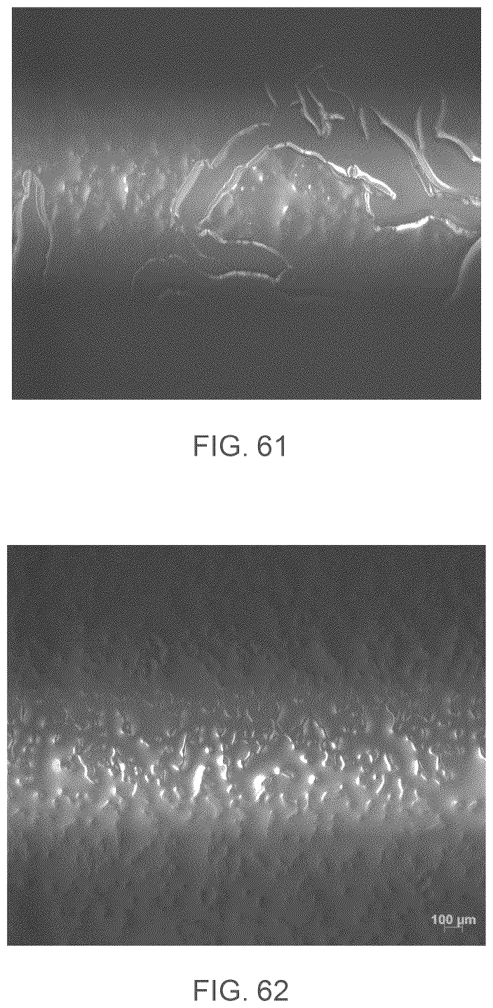

FIG. 61 reports an image showing the effect of an irradiation carried out for a time greater than the threshold of Is on a coating layer obtained according to Example B according to the invention.

As can be seen from the aforesaid figure, inhomogeneities extended up to a few millimetres that are comparable to grooves or lifting of the coating itself can be seen. Clearly the use of coating layers having a very low thickness (linked to the limited amounts of applied silicone material) emphasizes the occurrence of this effect.

FIG. 62 reports an image showing the effect of an irradiation equal to 0.3 seconds carried out on a coating layer obtained according to Example A according to the invention.

As can be seen from the aforesaid figure, the surface of the coating layer is characterized by a much finer inhomogeneity in the distribution of the coating, with micrometric-sized peaks and valleys and does not have the defects detectable in FIG. 61.

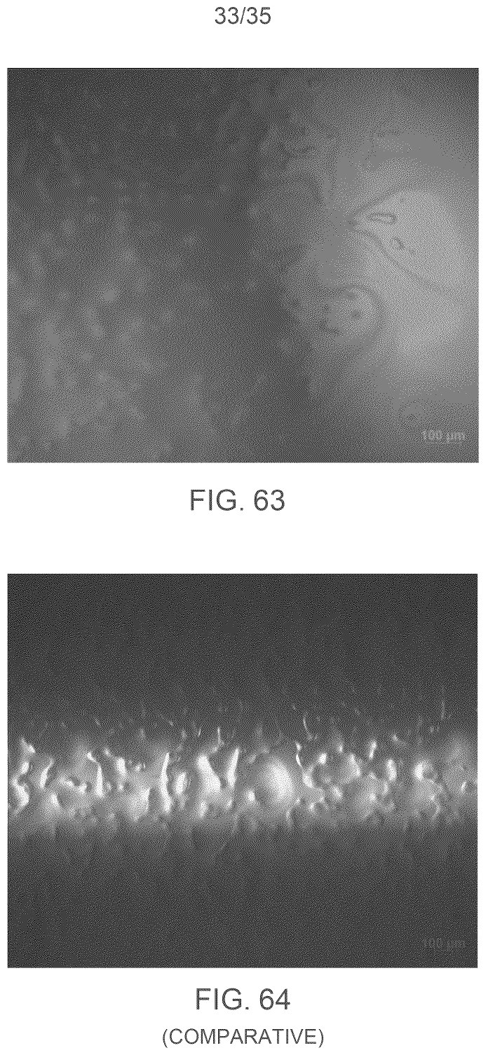

FIG. 63 reports an image showing the zone near the conical end portion of the cylinder of the same syringe as per FIG. 62.

As can be seen from FIG. 63, the surface of the coating layer is substantially homogeneous and substantially free of defects.

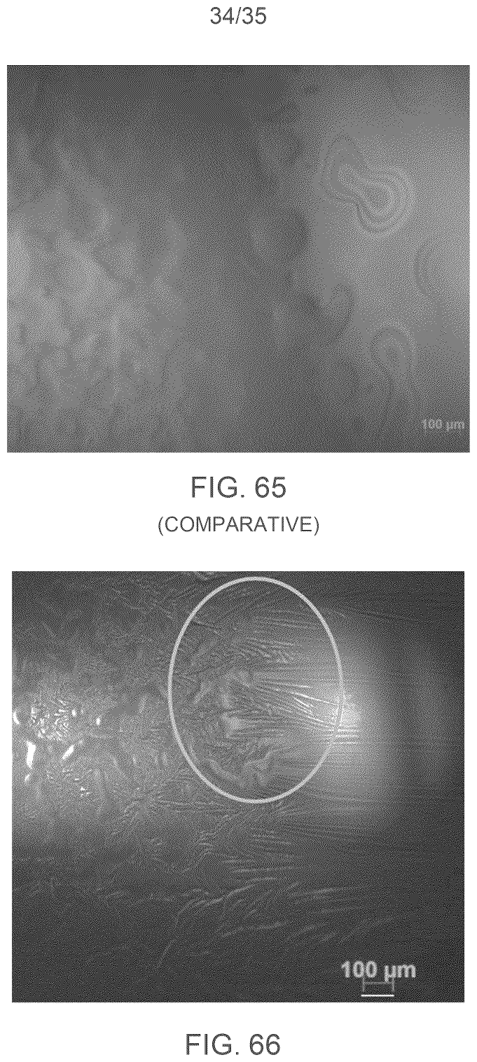

FIGS. 64 and 65 report images showing the effect of an irradiation equal to 0.3 seconds carried out on a coating layer obtained according to comparative Example C.

As can be seen from the aforesaid figures, taken in the cylindrical portion and, respectively, in the adjacent conical end portion of the cylinder, the surface of the coating layer is characterized by a greater granularity than that of the syringes according to the invention (Example A) as per the previous FIGS. 62 and 63.

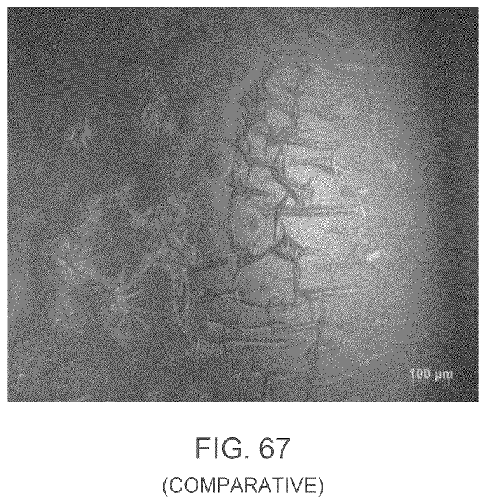

FIGS. 66 and 67 report images showing the effect of an irradiation close to the 1-second limit carried out on a coating layer obtained according to Example A according to the invention and according to comparative Example C in the connection zone between the cylindrical portion and the cone-shaped portion of the syringe cylinder.

As can be seen from FIGS. 66 and 67, by carrying out a plasma irradiation of the coating layer obtained according to Example A according to the invention (FIG. 66), it can be observed in the zone on the right of the image (cone-shaped portion of the cylinder) the presence of streaks even if not very marked.

However, the streaks appear much more marked, with the same radiation conditions, in the case of a coating layer obtained according to Comparative example C as shown in FIG. 67.

The preferred embodiments of the disclosure have been described above to explain the principles of the present disclosure and its practical application to thereby enable others skilled in the art to utilize the present disclosure. However, as various modifications could be made in the constructions and methods herein described and illustrated without departing from the scope of the present disclosure, it is intended that all matter contained in the foregoing description or shown in the accompanying drawings, including all materials expressly incorporated by reference herein, shall be interpreted as illustrative rather than limiting. Thus, the breadth and scope of the present disclosure should not be limited by the above-described exemplary embodiment but should be defined only in accordance with the following claims appended hereto and their equivalents.

1-59. (canceled)

60. A method of manufacturing a medical injection device comprising a glass cylinder having an inner surface coated with a coating layer, the cylinder being configured to receive a plunger with sliding engagement, the method comprising the steps of:
providing a coating composition comprising an amount equal to or greater than 92% by weight of polydimethylsiloxane having a kinematic viscosity at room temperature of from 11500 cSt (115 cm²/s) to 13500 cSt (135 cm²/s);
heating the coating composition to a temperature of from 100° C. to 150° C.;
applying the coating composition heated to said temperature onto the inner surface of the cylinder so as to form a coating layer having an average thickness S, measured by optical reflectometry, of from 100 to 250 nm on said inner surface; and
wherein the coating layer of the inner surface of the cylinder has a thickness standard deviation, equal to or less than 90 nm.

61. The method according to claim 60, wherein said step a) of providing the coating composition comprises storing said coating composition in a storage tank.

62. The method according to claim 61, wherein said step b) of heating the coating composition comprises heating said storage tank so as to bring the coating composition to said temperature of from 100° C. to 150° C.

63. The method according to claim 61, further comprising a step d) of maintaining the heated coating composition stored in the storage tank at a pressure of from 5 psi (0.34 bar) to 150 psi (10.34 bar).

64. The method according to claim 60, further comprising a step e) of feeding the heated coating composition to a dispensing head provided with at least one dispensing nozzle.

65. The method according to claim 64, wherein said step e) of feeding the heated coating composition to the dispensing head is carried out by means of a circulation pump arranged upstream of the dispensing head.

66. The method according to claim 64, wherein said step c) of applying the heated coating composition onto the inner surface of the cylinder is carried out by dispensing the coating composition via the dispensing head.

67. The method according to claim 65, wherein said step b) of heating the coating composition comprises heating said dispensing head and/or said pump so as to bring or maintain the coating composition to/at said temperature of from 100° C. to 150° C.

68. The method according to claim 61, wherein said storage tank, said pump and said dispensing head are in fluid communication by means of pipes and wherein said step b) of heating the coating composition comprises heating said pipes so as to bring or maintain the coating composition to/at said temperature of from 100° C. to 150° C.

69. The method according to claim 64, wherein said step c) of applying the heated coating composition onto the inner surface of the cylinder is carried out by dispensing the heated coating composition from the dispensing head at a pressure of from 5 psi (0.34 bar) to 150 psi (10.34 bar).

70. The method according to claim 64, wherein said step c) of applying the heated coating composition onto the inner surface of the cylinder comprises feeding to the dispensing head a dispensing gas having a pressure of from $ psi (0.34 bar) to 150 psi (10.34 bar).

71. The method according to claim 66, wherein said step c) of applying the heated coating composition onto the inner surface of the cylinder comprises imparting a relative motion between the dispensing head and the cylinder while dispensing the heated coating composition.

72. The method according to claim 71, wherein the step c) of applying the heated coating composition onto the inner surface of the cylinder comprises dispensing the heated coating composition onto the inner surface of the cylinder during a relative insertion movement of the dispensing head into the cylinder.

73. The method according to claim 71, the dispensing time of the heated coating composition onto the inner surface of the cylinder is of from 0.3 s to 1 s.

74. The method according to claim 60, wherein said step c) of applying the heated coating composition onto the inner surface of the cylinder comprises dispensing the heated coating composition at a flow rate of from 0.1 μL/s to 5 μL/s.

75. The method according to claim 60, wherein said step c) of applying the heated coating composition onto the inner surface of the cylinder comprises applying to the inner surface of the cylinder an amount per unit area of heated coating composition of from 0.2 to 0.4 μg/mm².

76. The method according to claim 60, further comprising, after step c) of applying the heated coating composition onto the inner surface of the cylinder, a step f) of subjecting the coating layer formed on the inner surface of the cylinder to a partial cross-linking treatment of the polydimethylsiloxane.

77. The method according to claim 76, wherein said partial cross-linking treatment is carried out by irradiation.

78. The method according to claim 77, wherein said irradiation treatment is a plasma irradiation treatment.

79. The method according to claim 77, wherein said irradiation treatment is an irradiation treatment by means of plasma torch at atmospheric pressure with argon flow.

80. The method according to claim 77, wherein said irradiation treatment is carried out for a time of from 0.2 s to 1 s, extremes included.

81. A medical injection device comprising a glass cylinder having an inner surface coated with a coating layer, the cylinder being configured to receive a plunger with sliding engagement,
wherein said coating layer of the inner surface of the cylinder is substantially made of polydimethylsiloxane having a kinematic viscosity at room temperature of from 11500 cSt (115 cm²/s) to 13500 cSt (135 cm²/s), and has an average thickness of from 100 to 250 nm; and
wherein the coating layer of the inner surface of the cylinder has a thickness standard deviation, equal to or less than 90 nm.

82. The medical injection device according to claim 81, wherein said coating layer of the inner surface of the cylinder is partially cross-linked.

83. The medical injection device according to claim 81, wherein the average value of the normalised concentration of the particles, released in a test solution from the coating layer of the inner surface of the cylinder, and having an average diameter equal to or greater than 10 μm or equal to or greater than 25 μm, determined by means of the LO (Light Obscuration) method according to US standard USP 787 as described in US Pharmacopeia 44-NF39 (2021), after a 3-month storage at a temperature of −40° C., is equal to or less than 60% of the limit value according to said standard.

84. The medical injection device according to claim 81, wherein the average value of the normalised concentration of the particles, released in a test solution from a partially cross-linked coating layer of the inner surface of the cylinder, and having an average diameter equal to or greater than 10 μm or equal to or greater than 25 μm, determined by means of the LO (Light Obscuration) method according to US standard USP 787 as described in US Pharmacopeia 44-NF39 (2021), after a 3-month storage at a temperature of −40° C., is equal to or less than 10% of the limit value according to said standard.

85. The medical injection device according to claim 81, wherein the average value of the normalised concentration of the particles, released in a test solution from a partially cross-linked coating layer of the inner surface of the cylinder, and having an average diameter equal to or greater than 10 μm or equal to or greater than 25 μm, determined by means of the LO (Light Obscuration) method according to US standard USP 789 as described in US Pharmacopeia 44-NF39 (2021), after a 3-month storage at a temperature of +5° C. or +25° C. or +40° C., is equal to or less than the limit value according to said standard.

86. A kit of parts for assembling a medical injection device comprising the following separate components in a sterile package:
a glass cylinder having an inner surface coated with a coating layer, the cylinder being configured to receive a plunger with sliding engagement;
a plunger configured for a sliding engagement in said cylinder;
wherein said coating layer of the inner surface of the cylinder is substantially made of polydimethylsiloxane having a kinematic viscosity at room temperature of from 11500 cSt (115 cm²/s) to 13500 cSt (135 cm²/s) and has an average thickness of from 100 to 250 nm; and
wherein the coating layer of the inner surface of the cylinder has a thickness standard deviation, equal to or less than 90 nm.
